(19) United States
(12) Patent Application Publication
(10) Pub. No.: US 2012/0101147 A1
(43) Pub. Date: Apr. 26, 2012

(54) INHIBITION OF HDAC2 TO PROMOTE MEMORY

(75) Inventors: Li-Huei Tsai, Cambridge, MA (US); Andre Fischer, Goettingen (DE); Stephen J. Haggarty, Dorchester, MA (US); Weiping Tang, Middleton, WI (US); Stuart L. Schreiber, Boston, MA (US); Edward Holson, Newton Highlands, MA (US); Florence Wagner, Ashland, MA (US); Mikel P. Moyer, Brookline, MA (US)

(73) Assignee: The General Hospital Corporation d/b/a Massachusetts General Hospital, Boston, MA (US)

(21) Appl. No.: 13/132,179

(22) PCT Filed: Dec. 2, 2009
(86) PCT No.: PCT/US09/06355

§ 371 Date: Nov. 14, 2011

Related U.S. Application Data
(62) Division of application No. 61/119,698, filed on Dec. 3, 2008.

(51) Int. Cl. A61K 31/7088; A61K 31/496; A61K 31/4436; A61P 25/28; A61K 31/381; A61K 31/18; A61K 31/16; A61K 31/165; A61P 25/00; A61K 31/44

(52) U.S. Cl. 514/44 A; 514/44 R; 514/252.13; 514/253.01; 514/336; 514/357; 514/438; 514/603; 514/616; 514/619

(57) ABSTRACT

The invention relates to methods and products for enhancing and improving recovery of lost memories. In particular the methods are accomplished by inhibiting HDAC2 and or selectively inhibiting HDAC1/2 or HDAC1/2/3.

### RELATED APPLICATION

This application claims priority under 35 USC §119 to U.S. Provisional Application No. 61/119,698, filed Dec. 3, 2008, the entire contents of which is hereby incorporated by reference.

### FEDERALLY SPONSORED RESEARCH

This invention was made with Government support under NIH NS051874. Accordingly, the Government has certain rights in this invention.

### BACKGROUND OF INVENTION

Brain atrophy occurs during normal aging and is an early feature of neurodegenerative diseases associated with impaired learning and memory. Only recently have mouse models with extensive neurodegeneration in the forebrain been reported (1-3). One of these models is the bi-transgenic CK-p25 Tg mice where expression of p25, a protein implicated in various neurodegenerative diseases (4), is under the control of the CamKII promoter and can be switched on or off with a doxycycline diet (3,5). Post-natal induction of p25 expression for 6 weeks caused learning impairment that was accompanied by severe synaptic and neuronal loss in the forebrain. However, pre-clinical research has not yet explored strategies to recover lost memories after substantial neuronal loss had taken place.

Neuronal adaptive responses, implicated in memory formation and storage, involve functional and structural synaptic changes, which require alterations in gene expression (West, A. E. et al. Proc Natl Acad Sci USA 98 (20), 11024-11031 (2001); Guan, Z. et al. Cell 111 (4), 483-493 (2002)). The mechanisms underlying this process are still unclear. Chromatin remodeling, especially through histone-tail acetylation, which alters the compact chromatin structure and changes the accessibility of DNA to regulatory proteins, is emerging as a fundamental mechanism for regulation of gene expression in development and adulthood (Kurdistani, S. K. & Grunstein, M. Nat Rev Mol Cell Biol 4 (4), 276-284 (2003); Goldberg, A. D., Allis, C. D., & Bernstein, E. Cell 128 (4), 635-638 (2007)).

### SUMMARY OF INVENTION

Neurodegenerative diseases of the central nervous system are often associated with impaired learning and memory, eventually leading to dementia. An important aspect that has not been addressed extensively in pre-clinical research, is the loss of long-term memories and the exploration of strategies to re-establish access to those memories. In some embodiments the current invention provides methods for restoring access to long-term memory after synaptic and neuronal loss has already occurred. Environmental enrichment (EE) has been shown to reinstate learning behavior and re-establish access to long-term memories after significant brain atrophy and neuronal loss has already occurred. Also shown herein is a correlation between EE and epigenetic changes. EE increases histone-tail acetylation and changes the level of methylation. The increase in acetylation and change in level of methylation is observed in hippocampal and cortical histone 3 (H3) and histone 4 (H4). In turn, elevated histone H3 and H4 acetylation initiate rewiring of the neural network.

In some aspects the invention is a method for enhancing a memory in a subject by administering to the subject an HDAC2 inhibitor in an amount effective to enhance the memory in the subject. The HDAC2 inhibitor may be a selective HDAC2 inhibitor. In other embodiments the HDAC2 inhibitor is non-selective but is not an HDAC1, HDAC5, HDAC6, HDAC7 and/or HDAC10 inhibitor. In yet other embodiments the HDAC2 inhibitor is an HDAC1/HDAC2 selective inhibitor or an HDAC1/HDAC2/HDAC3 selective inhibitor.

In some embodiments the invention provides a method for accessing long-term memory in a subject having diminished access to a long-term memory comprising increasing histone acetylation in an amount effective to reestablish access to long-term memory in the subject.

In some aspects of the invention the long-term memory is impaired. In some embodiments the impairment may be age-related or injury-related. In some embodiments of the invention a synaptic network in the subject is re-established. In some embodiments re-establishing the synaptic network comprises an increase in the number of active brain synapses. In some embodiments re-establishing the synaptic network comprises a reversal of neuronal loss. In some embodiments the subject has a disorder selected from the group consisting of MCI (mild cognitive impairment), Alzheimer's Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, vascular dementia, progressive supranuclear palsy or cortical basal degeneration.

The methods optionally involve administration of additional compounds. For instance, in some embodiments a HDAC3 inhibitor is administered. In other embodiments a HDAC11 inhibitor is administered. In yet other embodiments a DNA methylation inhibitor such as 5-azacytidine, 5-aza-2′deoxycytidine, 5,6-dihydro-5-azacytidine, 5,6-dihydro-5-aza-2′deoxycytidine, 5-fluorocytidine, 5-fluoro-2′deoxycytidine, and short oligonucleotides containing 5-aza-2′deoxycytosine, 5,6-dihydro-5-aza-2′deoxycytosine, and 5-fluoro-2′deoxycytosine, and procainamide, Zebularine, and (−)-egallocatechin-3-gallate is administered. An additional therapeutic agent such as ARICEPT or donepezil, COGNEX or tacrine, EXELON or rivastigmine, REMINYL or galantamine, anti-amyloid vaccine, Abeta-lowering therapies, mental exercise or stimulation may be administered.

In other embodiments the HDAC2 inhibitor is an HDAC2 RNAi such as a siRNA, shRNA, miRNA, dsRNA or ribozyme or variants thereof.

The HDAC2 inhibitor may be administered orally, intravenously, cutaneously, subcutaneously, nasally, imtramuscularly, intraperitoneally, intracranially, or intracerebroventricularly.

The methods may also include a step of assessing cognitive function of the subject after administration of the HDAC2 inhibitor. Further the method may involve monitoring treatment by assessing cerebral blood flow or blood-brain barrier function.

A method for treating Alzheimer's disease by administering to a subject having Alzheimer's disease an HDAC2 inhibitor in an amount effective to treat Alzheimer's disease is provided according to other aspects of the invention. In one embodiment the HDAC2 inhibitor is a selective HDAC2 inhibitor.

In some embodiments the HDAC2 inhibitor is a selective HDAC1/HDAC2 inhibitor. In other embodiments the HDAC2 inhibitor is a selective HDAC1/HDAC2/HDAC3 inhibitor. In some embodiments, the HDAC2 inhibitor is a selective HDAC1/HDAC2/HDAC10 inhibitor. In some embodiments, the selective HDAC1/HDAC2/HDAC10 inhibitor is BRD-6929. In other embodiments, the HDAC2 inhibitor is a selective HDAC1/HDAC2/HDAC3/HDAC10 inhibitor.

In yet other embodiments the HDAC2 inhibitor is a compound of formula (IV)

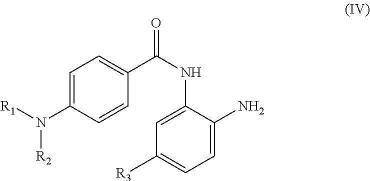

wherein R₁ and R₂ are independently selected from H, and —C(O)—C₁₋₆alkyl; R₃ is optionally substituted aryl, optionally substituted heteroaryl, or aryl-C₁₋₆alkylene.

In some embodiments R₁ is H; R₁ and R₂ are H; R₁ is —C(O)—C₁₋₆alkyl; R₁ is —C(O)-methyl; R₁ is —C(O)-methyl and R₂ is H; R₃ is optionally substituted aryl; R₃ is tolyl; R₃ is optionally substituted heteroaryl; R₃ is thienyl; R₃ is aryl-C₁₋₆alkylene; or R₃ is phenyl-ethylene.

In other embodiments formula IV is

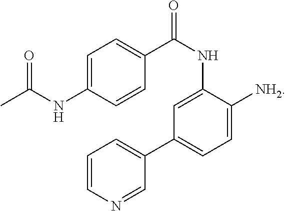

In other embodiments formula IV is

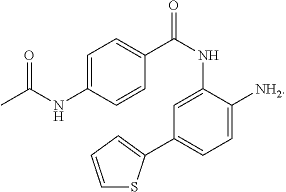

In other embodiments formula IV is

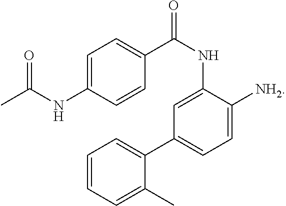

In other embodiments formula IV is

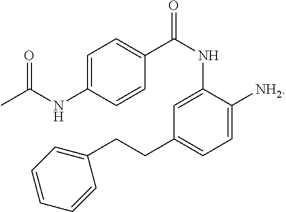

The HDAC2 inhibitor in other embodiments is a compound of formula (VI)

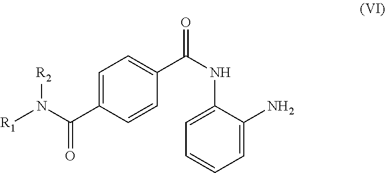

wherein R₁ and R₂ are independently selected from H, substituted or unsubstituted, branched or unbranched, cyclic or acyclic C₁₋₆alkyl, heterocyclyl, heteroaryl, aryl, and aryl-C₁₋₆alkylene.

In some embodiments R₁ is H; R₁ and R₂ are H: R₁ is methyl, ethyl, propyl, or butyl; R₁ is aryl-C₁₋₆alkylene; R₁ is phenyl-ethylene; or R₂ is H.

In other embodiments formula VI is

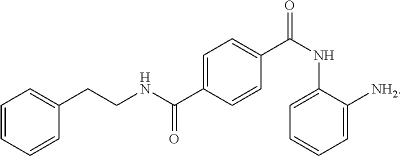

The HDAC2 inhibitor in other embodiments is a compound of formula (I)

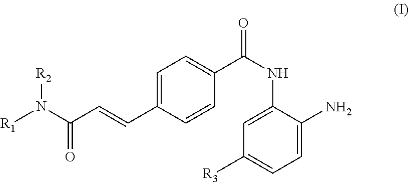

wherein R₁ and R₂ are independently selected from H, substituted or unsubstituted, branched or unbranched, cyclic or acyclic C₁₋₆alkyl, heterocyclyl, C₁₋₆alkylene, heteroaryl, heteroarylene, and heteroarylene-alkylene; and R₃ is aryl or heteroaryl.

In some embodiments R₁ is unsubstituted acyclic C₁₋₆alkyl; R₁ is selected from a group consisting of methyl, ethyl, propyl, and butyl; R₁ is heteroarylene-alkylene; R₁ is heteroarylene-C₁₋₆alkylene; R₁ is pyridinyl-ethylene; R₂ is hydrogen; R₃ is heteroaryl; or R₃ is thienyl.

In yet other embodiments formula I is

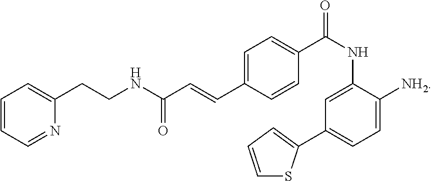

The HDAC2 inhibitor in some embodiments is a compound of formula (II)

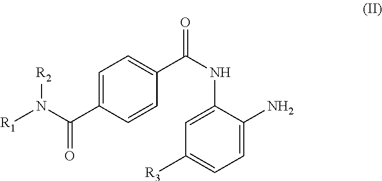

wherein R₁ and R₂ are independently selected from H, substituted or unsubstituted, branched or unbranched, cyclic or acyclic C₁₋₆alkyl, heterocyclyl, C₁₋₆alkylene, heteroaryl, heteroarylene, heteroarylene-alkylene, arylene-alkylene; and heterocyclyl-alkylene optionally substituted; and R₃ is aryl or heteroaryl.

In some embodiments R₁ is unsubstituted acyclic C₁₋₆alkyl; R₁ is selected from a group consisting of methyl, ethyl, propyl, and butyl; R₁ is heteroarylene-alkylene; R₁ is heteroarylene-C₁₋₆alkylene; R₁ is pyridinyl-ethylene; R₁ is arylene-alkylene; R₁ is arylene-C₁₋₆alkylene; R₁ is phenyl-ethylene; R₁ is heterocyclyl-alkylene; R₁ is unsubstituted heterocyclyl-C₁₋₆alkylene; R₁ is piperazine-ethylene; R₁ is substituted heterocyclyl-C₁₋₆alkylene; R₁ is substituted piperazine-ethylene; R₁ is C₁₋₆alkylene substituted piperazine-ethylene; R₁ is methyl substituted piperazine-ethylene; R₂ is hydrogen; R₃ is heteroaryl; R₃ is thienyl; or R₃ is pyridinyl.

In other embodiments formula II is

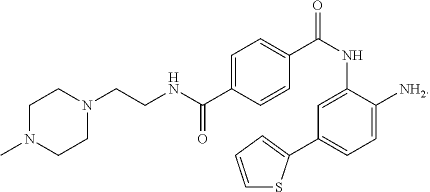

In other embodiments formula II is

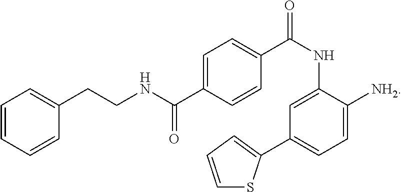

In other embodiments formula II is

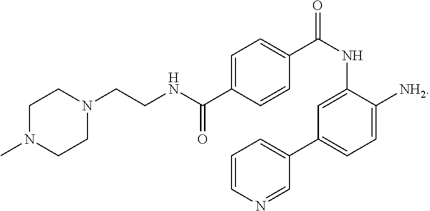

In other embodiments formula II is

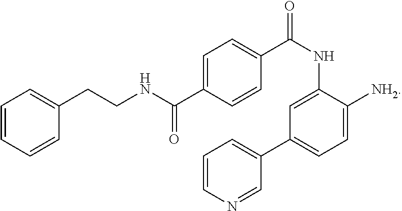

In other embodiments formula II is

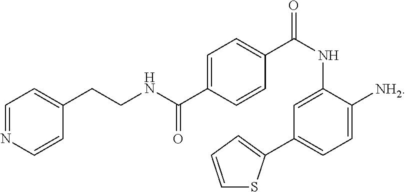

The HDAC2 inhibitor in some embodiments is a compound of formula (III)

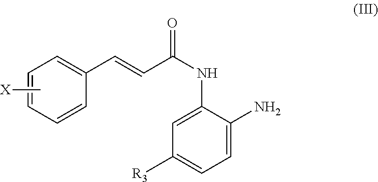

wherein X is —C(O)—N(R₁)(R₂), C₁₋₆alkylene-N(H)—C₁₋₆alkylene-N(R₁)C(O)(R₂); or —N(R₁)C(O)R₂; R₁ and R₂ are independently selected from H, and substituted or unsubstituted, branched or unbranched, cyclic or acyclic C₁₋₆alkyl; and R₃ is alkynyl, aryl, or heteroaryl.

In some embodiments X is —C(O)—N(R₁)(R₂); R₁ and R₂ are independently selected from H, unsubstituted, unbranched, acyclic C₁₋₆alkyl; R₁ and R₂ are independently selected from H, methyl, ethyl, propyl, and butyl; R₁ is H; R₁ and R₂ are H; X is —C(O)—NH₂; X is C₁₋₆alkylene-N(R₁)—C₁₋₆alkylene-N(R₁)C(O)(R₂); R₁ is H; X is C₁₋₆alkylene-N(H)—C₁₋₆alkylene-N(H)C(O)(R₂); X is —N(R₁)C(O)R₂; R₁ is H; R₁ is unsubstituted acyclic C₁₋₆alkyl; R₁ is selected from a group consisting of methyl, ethyl, propyl, and butyl; R₂ is unsubstituted acyclic C₁₋₆alky; R₂ is selected from a group consisting of methyl, ethyl, propyl, and butyl; R₃ is heteroaryl; R₃ is thienyl; R₃ is aryl; R₃ is alkynyl; R₃ is C₁₋₆alkynyl; or R₃ is ethynyl.

In some embodiments formula III is

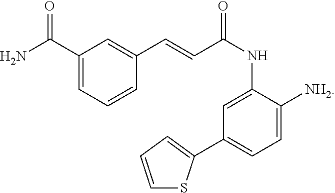

In some embodiments formula III is

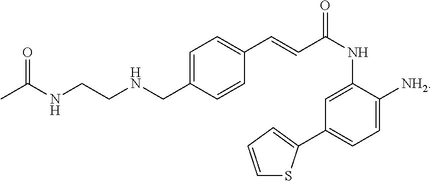

In some embodiments formula III is

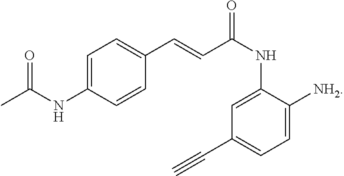

The HDAC2 inhibitor in other embodiments is a compound of formula (V)

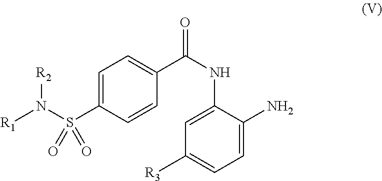

wherein R₁ and R₂ are independently selected from H, and substituted or unsubstituted, branched or unbranched, cyclic or acyclic C₁₋₆alkyl; and R₃ is aryl or heteroaryl.

In some embodiments R₁ is H; R₁ and R₂ are H; R₁ is methyl, ethyl, propyl, or to butyl; R₃ is aryl; R₃ is heteroaryl; or R₃ is thienyl.

In other embodiments formula V is

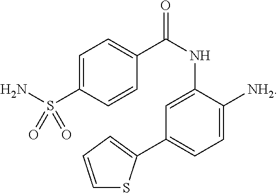

In some embodiments the methods specifically exclude the use of molecules of Formula IV.

Pharmaceutical compositions of a HDAC2 inhibitor and a pharmaceutically acceptable carrier in a formulation for delivery to brain tissue are also provided. In some embodiments the HDAC2 inhibitor is formulated for crossing blood brain barrier.

In other aspects the invention is a composition of an HDAC2 inhibitor, wherein the HDAC2 inhibitor is selected from the group consisting of compounds of formula I, II and III.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 shows HDAC inhibitor improved associative learning via HDAC2. a. Memory test for mice with contextual fear conditioning training (foot shock 1.0 mA). HDAC2OE mice (SAHA group, n=12; saline group, n=12) and WT littermates (SAHA group, n=12; saline group, n=15) were treated with saline or SAHA (25 mg/kg, i.p.) for 10 days before memory test. b. CA1 region (pyramidal neuron layer; stratum radiatum (s.r.)) from WT and HDAC2OE mice received chronic SAHA treatment or saline treatment and were observed through immunostaining. Average optical signals for Ac-lysine were measured on pyramidal neuron layer; SVP signals were measured from s.r. c. Images of Golgi staining from CA1 region of hippocampus. For WT, naive, n=23; WT, SAHA, n=41; HDAC2OE, naïve, n=21; HDAC2OE, SAHA, n=32. Scale bar 10 μm. d. Memory test for mice with contextual fear conditioning training (foot shock 0.5 mA) after 10 day SAHA injection (25 mg/kg, i.p.). WT mice (SAHA, n=10; saline, n=10) and HDAC2 KO mice (SAHA, n=8; saline, n=8). e. CA1 region from HDAC2KO mice received chronic SAHA treatment or saline treatment and were observed through immunostaining. SVP-singles were quantified in the s.r. Saline, n=15; SAHA, n=22. Scale bar=50 μm. f. Images of Golgi staining of CA1 region of hippocampus from HDAC2KO mice. HDAC2KO, SAHA, n=24; HDAC2KO, naïve, n=27. *, p<0.05; **, p<0.005; ***,p<0.001, unpaired student t-test error bars indicate s.e.m.

FIG. 2 Increased α-Tubulin(K40) acetylation resulting from HDAC6 inhibition does not facilitate associative learning in mice. a. The structure of WT-161 is shown. b. Selectivity of WT-161 (2 μM) for increasing acetylated α-tubulin(K40) over total acetylated lysine (Ac-lysine) was measured in human MM1.S cells treated for 16 hrs and assessed for hyperacetylated histones and/or α-tubulin(K40) using quantitative immunofluorescence imaging. Data presented are derived from a primary screen of a library of compounds biased for deacetylase function. c. Immunostaining of acetylated α-tubulin(K40) in area CA1 of hippocampus from mice treated with WT-161 or SAHA (both conditions in 25 mg/kg, i.p., 10 days) or saline is shown. Acetylated α-tubulin(K40) immunoreactive intensity signals in area CA1 were quantified (n=9, for each group). **, p<0.005. d. Memory test of WT mice injected with SAHA (25 mg/kg) or WT-161 (25 mg/kg) for 10 days. Mice were subjected to contextual fear conditioning training 24 hours before test (WT, n=20; SAHA, n=20; WT-161, n=10; ***, p<0.0005, student t-test).

FIG. 3 Expression and distribution of HDAC1 and HDAC2 in HDAC1OE and HDAC2OE mouse brain. a. Representative immunostaining images showing the expression of HDAC1 in the WT and HDAC1OE mice brain are provided. In WT brain, HDAC1 expression level is relatively higher in dentate gyrus than other areas of the brain. Increased HDAC1 signal in HDAC1OE brain is detected not only in the hippocampus but also in the cortex, amygdala (indicated with dashed lines) and basal forebrain. b. Representative immunostaining images showing the expression of HDAC2 in WT and HDAC2OE mice brain are presented. Scale bar, 400 μm. Scale bar for insertion, 100 μm.

FIG. 4 HDAC2KO mice exhibit enhanced memory in behavior tasks. a. Escape latency of WT, HDAC1OE and HDAC2OE mice in the visible platform water maze test. Mice were trained in the swimming pool with a visible platform for 3 days, with two trials per training day. The latency for mice to reach the platform was quantified (n=8 for each group). All three groups of mice reached the platform with similar escape latencies on the first day. No significant difference in escape latency was detected between the three groups of mice during the 3 days of training. b. Swimming speed in the water maze pool (n=8 for each group) is shown. c-d. Short-term memory was tested for WT, HDAC1OE and HDAC2OE mice in contextual- and tone-dependent fear conditioning paradigms (WT, n=9; HDAC2OE, n=9; HDAC1OE, n=8). No significant difference was detected between the WT group and the HDAC1/2OE mice. e-f. Short-term memory was tested for HDAC2KO mice in contextual- and tone-dependent fear conditioning paradigms (WT, n=8; HDAC2KO, n=9). HDAC2KO mice showed significantly increased freezing in contextual fear conditioning (p=0.0100, compared to WT littermates), but not in tone-dependent fear conditioning (p=0.1439). g. Mean percent correct responses for WT (n=8) and HDAC2KO mice (n=10) during spatial non-matching to place testing on the elevated T-maze is shown. HDAC2KO mice showed significant higher accuracy during the training period (Block 2, p=0.044, Block 3, p=0.0087, student t-test; between genotypes, p=0.0252, two-way ANOVA). h. Mean percent correct responses for WT (n=8), HDAC1OE (n=7) and HDAC2OE (n=9) mice during spatial non-matching to place testing on the elevated T-maze is shown. HDAC2OE mice showed significant defects in accuracy during training trail block 2 (p=0.0452, student t-test).

FIG. 5 Characterization of HDAC2KO mice. a. Schematic representation of the murine Hdac2 genomic locus is shown. Gray filled boxes indicate exons. Black arrowheads indicate loxP positions. P14F, P15R and P2 are oligo DNA primers used for genotyping. b. Westernblot analysis of protein lysates obtained from wild-type, Hdac2^{L/+} and Hdac2^{L/L} MEFs infected with either vector (V) or Cre-recombinase expressing retroviruses, using HDAC2 specific antibodies was performed. Cdk4 served as a loading control. c. Observed and expected numbers and frequencies of wild-type, Hdac2^{+/-} and Hdac2^{-/-} mice obtained from multiple Hdac2^{+/-} intercrosses. d. Western blot analysis of HDAC1 and HDAC2 expression levels in the brain lysate from the Hdac2^{-/-} mouse and WT littermate was performed. HDAC1 expression level was increased in Hdac2^{-/-} mice.

FIG. 6 SAHA treatment facilitates LTP in WT but not HDAC2KO hippocampus. a-b. One-month-old HDAC2KO mice and their WT littermates were injected with SAHA (25 mg/kg, i.p.) or saline for 10 days. An additional injection was introduced 30 minutes before sacrifice. Long-term potentiation (LTP) was induced by one HFS stimulation (1×100 Hz, 1 s) of Schaffer collaterals. a. A significant increase in the magnitude of LTP was observed in the SAHA treated WT mice when compared to the saline group. b. No significant difference in the magnitude of LTP was detected between SAHA and saline treated HDAC2KO mice. (**, p<0.005, two-way ANOVA).

FIG. 7 is a bar graph depicting the results of in vitro assays testing the protective effects of HDAC over expression on p25 induced toxicity. Neurons were dissociated from E15.5 cortex and hippocampus and transfected with plasmids encoding p25-GFP and Flag-HDACs at DIV4. 24 hrs after transfection, neurons were fixed and processed for IHC. All p25 positive neurons were counted, assuming most neurons are transfected by both p25 and HDACs.

FIG. 8 is a table which shows the enzymatic inhibitory activity of multiple HDAC inhibitors against several of the known HDAC isoforms.

FIG. 9 shows the effects of HDAC inhibitors on histone acetylation marks in HeLa cell lysate. Series of compounds incubated with whole HEK293 cells at 10 uM for a 6 hour time period. Western blot showing increased acetylation levels over DMSO controls using anti-acetyl H4K12 antibodies and horseradish peroxidase conjugated secondary antibody along with a luminol-based substrate. This demonstrates cellular HDAC activity of these analogs and the increase in acetylation in the specific mark, H4K12.

FIG. 10 is the quantification of the raw western data shown in FIG. 9. Relative to the DMSO control, multiple selectivity profiles are effective in increasing H4K12 acetylation levels. This demonstrates that HDAC 1,2 and HDAC 1,2,3 selective inhibitors have robust HDAC activity in whole cells on a specific histone loci (H4K12). BRD-9853 shows minimal activity in this cell line. BRD-4097 is the negative control. This is a benzamide with minimal HDAC inhibitory activity.

FIG. 11 is the quantification of the raw western blots used to measure the effects of HDAC inhibitors on histone acetylation marks in HeLa cell lysate. Relative to the DMSO control, there are varying degrees of acetylation. The histogram demonstrates that HDAC1,2 and HDAC1,2,3 selective compounds are effective at increasing the acetylation at the H4K12 loci.

FIG. 12 shows the increased H4K12 acetylation in mouse primary striatal cells. A. Western blots of primary striatal cells isolated from mouse brain that have been treated with HDAC inhibitors. Two sets of data with 3 independent samples/set. B. Histograms represent the quantification of westerns shown in panel A.

FIG. 13 shows that treatment of neuronal cells with BRD-6929 and BRD-5298 enhances H4 and H2B histone acetylation in vitro.

FIG. 14 demonstrates the nuclear intensity of increased H4K12-acetylation in mouse primary neuronal cultures. A. Control demonstrating that BRD-6929 at 1 and 10 uM does not cause an increase or decrease in overall cell number after 6 h incubation in brain region specific primary cultures (cortex and striatum). B. Histograms showing that BRD-6929 at 10 uM causes an increase in H4K12 acetylation after 6 h incubation in to brain region specific primary cultures (striatum). Thus, An HDAC 1,2 selective compound is effective at increasing acetylation at a specific histone locus (H4K12) in cultured striatal neurons.

FIG. 15 demonstrates that an HDAC 1,2 selective compound can significantly increase acetylation marks associated with memory and learning in neuronal cells isolated from specific brain regions and analyzed using immunofluorescence. A. Control demonstrating that BRD-6929 at 1 and 10 uM does not cause an increase or decrease in overall cell number after 6 h incubation in brain region specific primary cultures (striatum). B. Histograms showing that BRD-6929 at 1 and 10 uM causes a 2-3 fold increase in H2B tetra-acetylation after 6 h incubation in brain region specific primary cultures (striatum). This effect is significant relative to the DMSO control in all instances.

FIG. 16 demonstrates that HDAC 1,2 selective compounds are effective in increasing the acetylation at the specific histone locus H2B. A. Micrograph showing the increased fluorescence in primary neuronal cells after treatment with DMSO or 10 uM BRD-5298, an HDAC 1,2 selective inhibitor, after 6 h incubation. The increased magenta fluorescence corresponds to increased levels of H2B acetylation. B. Control demonstrating that BRD-6929 and BRD-5298 at 1 and 10 uM do not cause an increase or decrease in overall cell number after 6 h incubation in primary neuronal cell cultures. C. Histograms showing that BRD-6929 and BRD-5298 (HDAC1,2 selective inhibitors) at 1 and 10 uM cause a significant increase in H2B acetylation after 6 h incubation in primary neuronal cell cultures.

FIG. 17 is the concentration-time curve of BRD-6929 in plasma and brain following single 45 mg/kg i.p. dose in mice.

FIG. 18 is the experimental protocol for acute treatment with BRD-6929 and the corresponding effects on histone acytlation in brain specific regions of adult male C57BL/6J mice.

FIG. 19 shows that acute treatment with BRD-6929 causes H2B (tetra) histone acetylation in cortex of adult male C57BL/6J mice. The histograms on the left are the quantification of the western gel data shown on the right. The data has been normalized to the level of histone H3 levels. BRD-6929 causes a 1.5-2 fold increase in cortex for this mark. This demonstrates that BRD-6929 is a functional inhibitor of HDACs in the cortex after a single dose given systemically.

FIG. 20 shows that acute treatment with BRD-6929 causes increased H2BK5 histone acetylation in cortex of adult male C57BL/6J mice. In cortex after 1 hour, BRD-6929 causes a 1.5-2 fold increase in the acetylation levels for H2BK5. This acetylation mark has been associated with increased learning and memory.

FIG. 21 demonstrates the increase in acetylation marks in whole brain after chronic administration of BRD-6929.

FIG. 22 demonstrates that BD-6929 increased associative learning and memory in WT C57/BL6 mice.

### DETAILED DESCRIPTION

Increased histone-tail acetylation induced by inhibitors of histone deacetylases (HDACis) facilitates learning and memory in wildtype mice, as well as in mouse models of neurodegeneration. Harnessing the therapeutic potential of HDACis requires knowledge of the specific HDAC family members linked to cognitive enhancement. It is shown according to aspects of the invention that neuron-specific overexpression of HDAC2, but not HDAC1, reduced dendritic spine density, synapse number, synaptic plasticity, and memory formation. Conversely, HDAC2 deficiency resulted in increased synapse number and memory facilitation, similar to chronic HDAC inhibitor treatment in mice. Notably, reduced synapse number and learning impairment of HDAC2 overexpressing mice was completely ameliorated by chronic HDACi treatment. Correspondingly, HDACi treatment failed to further facilitate memory formation in HDAC2 deficient mice. Furthermore, analysis of promoter occupancy revealed HDAC2 associates with the promoter of genes implicated in synaptic plasticity and memory formation. Our results suggest that HDAC2 plays a role in modulating long lasting changes of the synapse, which in turn negatively regulates learning and memory.

The invention relates in some aspects to therapeutics for enhancing and/or retrieving memories as well as promoting learning and memory. A “memory” as used herein refers to the ability to recover information about past events or knowledge. Memories include short-term memory (also referred to as working or recent memory) and long-term memory. Short-term memories involve recent events, while long-term memories relate to the recall of events of the more distant past. Enhancing or retrieving to memories is distinct from learning. However, in some instances in the art learning is referred to as memory. The present invention distinguishes between learning and memory and is focused on enhancing memories. Learning, unlike memory enhancement, refers to the ability to create new memories that had not previously existed. In some instances the invention also relates to methods for enhancing learning. Thus in order to test the ability of a therapeutic agent to effect the ability of a subject to learn rather than recall old memories, the therapeutic would be administered prior to or at the same time as the memory is created. In order to test the ability of a therapeutic to effect recall of a previously created memory the therapeutic is administered after the memory is created and preferably after the memory is lost.

In some instances the invention relates to methods for recapturing a memory in a subject. In order to recapture the memory the memory has been lost. A lost memory is one which cannot be retrieved by the subject without assistance, such as the therapeutic of the invention. In other words the subject cannot recall the memory. As used herein the term “recapture” refers to the ability of a subject to recall a memory that the subject was previously unable to recall. Generally, such a subject has a condition referred to as memory loss. A subject having memory loss is a subject that cannot recall one or more memories. The memories may be short term memories or long term memories. Methods for assessing the ability to recall a memory are known to those of skill in the art and may include routine cognitive tests.

In other instances the invention relates to a method for accessing long-term memory in a subject having diminished access to a long-term memory. A subject having diminished access to a memory is a subject that has experienced one or more long term memory lapses. The long-term memory lapse may be intermittent or continuous. Thus, a subject having diminished access to a long term memory includes but is not limited to a subject having memory loss, with respect to long term memories.

In some instances the long-term memory of the “subject having diminished access” may be impaired. An impaired long-term memory is one in which a physiological condition of the subject is associated with the long-term memory loss. Conditions associated with long-term memory loss include but are not limited to age related memory loss and injury related memory loss.

As used herein “age related memory loss” refers to refers to any of a continuum of conditions characterized by a deterioration of neurological functioning that does not rise to the level of a dementia, as further defined herein and/or as defined by the Diagnostic and Statistical Manual of Mental Disorders: 4th Edition of the American Psychiatric Association (DSM-IV, 1994). This term specifically excludes age-related dementias such as Alzheimer's disease and Parkinson's disease, and conditions of mental retardation such as Down's syndrome. Age related memory loss is characterized by objective loss of memory in an older subject compared to his or her younger years, but cognitive test performance that is within normal limits for the subject's age. Age related memory loss subjects score within a normal range on standardized diagnostic tests for dementias, as set forth by the DSM-IV. Moreover, the DSM-IV provides separate diagnostic criteria for a condition termed Age-Related Cognitive Decline. In the context of the present invention, as well as the terms “Age-Associated Memory Impairment” and “Age-Consistent Memory Decline” are understood to be synonymous with the age related memory loss. Age-related memory loss may include decreased brain weight, gyral atrophy, ventricular dilation, and selective loss of neurons within different brain regions. For purposes of some embodiments of the present invention, more progressive forms of memory loss are also included under the definition of age-related memory disorder. Thus persons having greater than age-normal memory loss and cognitive impairment, yet scoring below the diagnostic threshold for frank dementia, may be referred to as having a mild neurocognitive disorder, mild cognitive impairment, late-life forgetfulness, benign senescent forgetfulness, incipient dementia, provisional dementia, and the like. Such subjects may be slightly more susceptible to developing frank dementia in later life (See also US patent application 2006/008517, which is incorporated by reference). Symptoms associated with age-related memory loss include but are not limited to alterations in biochemical markers associated with the aging brain, such as IL-1beta, IFN-gamma, p-JNK, p-ERK, reduction in synaptic activity or function, such as synaptic plasticity, evidenced by reduction in long term potentiation, diminution of memory and reduction of cognition.

As used herein “injury related memory loss” refers to damage which occurs to the brain, and which may result in neurological damage. Sources of brain injury include traumatic brain injury such as concussive injuries or penetrating head wounds, brain tumors, alcoholism, Alzheimer's disease, stroke, heart attack and other conditions that deprive the brain of oxygen, meningitis, AIDS, viral encephalitis, and hydrocephalus.

A subject shall mean a human or vertebrate animal or mammal including but not limited to a dog, cat, horse, cow, pig, sheep, goat, turkey, chicken, and primate, e.g., monkey. Subjects are those which are not otherwise in need of an HDAC inhibitor. Subjects specifically exclude subjects having Alzheimer's disease, except in the instance where a subject having Alzheimer's disease is explicitly recited.

The methods of the invention generally relate to methods for enhancing memories. Methods for enhancing memories include reestablishing access to memories as well as recapturing memories. The term re-establishing access as used herein refers to increasing retrieval of a memory. Although Applicants are not bound by a mechanism of action, it is believed that the compounds of the invention are effective in increasing retrieval of memories by re-establishing a synaptic network. The process of re-establishing a synaptic network may include an increase in the number of active brain synapses and or a reversal of neuronal loss.

As used herein, the term re-establish access to long-term memory when used with respect to a disorder comprising memory loss or memory lapse refers to a treatment which increases the ability of a subject to recall a memory. In some instances the therapeutic of the invention also decreases the incidence and/or frequency with which the memory is lost or cannot be retrieved.

A subject in need of enhanced memories is one having memory loss or memory lapse. The memory loss may occur by any mechanism, such as it may be age related or caused by injury or disorders associated with cognitive impairment. Disorders associated with cognitive impairment include for instance MC1 (mild cognitive impairment), Alzheimer's Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, vascular dementia, progressive supranuclear palsy or cortical basal degeneration.

Alzheimer's disease is a degenerative brain disorder characterized by cognitive and noncognitive neuropsychiatric symptoms, which accounts for approximately 60% of all cases of dementia for patients over 65 years old. In Alzheimer's disease the cognitive systems that control memory have been damaged. Often long-term memory is retained while short-term memory is lost; conversely, memories may become confused, resulting in mistakes in recognizing people or places that should be familiar. Psychiatric symptoms are common in Alzheimer's disease, with psychosis (hallucinations and delusions) present in many patients. It is possible that the psychotic symptoms of Alzheimer's disease involve a shift in the concentration of dopamine or acetylcholine, which may augment a dopaminergic/cholinergic balance, thereby resulting in psychotic behavior. For example, it has been proposed that an increased dopamine release may be responsible for the positive symptoms of schizophrenia. This may result in a positive disruption of the dopaminergic/cholinergic balance. In Alzheimer's disease, the reduction in cholinergic neurons effectively reduces acetylcholine release resulting in a negative disruption of the dopaminergic/cholinergic balance. Indeed, antipsychotic agents that are used to relieve psychosis of schizophrenia are also useful in alleviating psychosis in Alzheimer's patients and could be combined with the compositions described herein for use in the methods of the invention.

Methods for recapturing a memory in a subject having Alzheimer's disease by administering an HDAC inhibitor are also provided according to the invention. Such methods optionally administering the inhibitor and monitoring the subject to identify recapture of a memory that was previously lost. Subjects may be monitored by routine tests known in the art. For instance some are described in books such as DSM described above or in the medical literature.

Vascular dementia, also referred to as “multi-infarct dementia”, refers to a group of syndromes caused by different mechanisms all resulting in vascular lesions in the brain. The main subtypes of vascular dementia are, for example vascular mild cognitive impairment, multi-infarct dementia, vascular dementia due to a strategic single infarct (affecting the thalamus, the anterior cerebral artery, the parietal lobes or the cingulate gyrus), vascular dementia due to hemorrhagic lesions, small vessel disease (including, e.g. vascular dementia due to lacunar lesions and Binswanger disease), and mixed Alzheimer's Disease with vascular dementia.

HDACs interact with other chromatin-modifying enzymes and co-regulators and play a key role in shaping epigenetic landscapes (Goldberg, A. D., Allis, C. D., & Bernstein, E. Cell 128 (4), 635-638 (2007).). There are a total of 18 HDAC enzymes in the mammalian genome, which are generally divided into four classes including class I, II, III and IV. These enzymes are known to have both histone and non-histone substrates. With the exception of the class II HDAC5, which has recently been implicated in the response to both antidepressant action (Tsankova, N. M. et al. Nat Neurosci 9 (4), 519-525 (2006).) and chronic emotional stimuli (Renthal, W. et al. Neuron 56 (3), 517-529 (2007).), little is known about the function of HDACs in the brain. Among the HDACs, Class I, II and IV HDACs are the zinc-dependent hydrolases. Class I HDACs include 1, 2, 3, and 8, which have been well documented to exert deacetylase activity on histone substrates as well as non-histone substrates. These family members are all inhibited by the non-selective HDAC inhibitor sodium butyrate. Class II HDACs can be divided into Class IIa members, which include HDAC 4, 5, 7 and 9, and Class IIb members, which include HDAC6 and 10. In the case of HDAC5, a role in the brain has been identified in response to both antidepressant action and to chronic emotional stimuli. However, whether class IIa HDACs themselves have functional histone (or other non-histone) deacetylates activity, rather than activity contributed by co-purifying class I HDACs, currently remains unclear. Class IIb family members, HDAC6 and 10 are mainly localized in the cytoplasm. HDAC6 is unique in the family in its possession of two deacetylase domains. HDAC6 has been shown to function as both an α-tublin (K40) deacetylase and to regulate ubiquitin-dependent protein degradation by the proteasome. In contrast, class III HDACs (sirtuins; SIRT1-7) are non-classical, NAD(+)-dependent enzymes, which exhibit a non-overlapping sensitivity to most structural classes of inhibitors of zinc-dependent HDACs, including SB. The latter finding suggests the sirtuins are not the relevant targets of HDACi induced memory enhancement.

The compounds useful according to the invention are HDAC2 inhibitors. An HDAC2 inhibitor as used herein is any compound, including proteins, small molecules, and nucleic acids, that reduces HDAC2 activity and/or expression. HDAC2 inhibitors may in some embodiments be selective HDAC2 inhibitors. A selective HDAC2 inhibitor is a compound that inhibits the activity or expression of HDAC2 but does not significantly inhibit the activity or expression of at least 2 other HDAC enzymes such as HDAC1, HDAC3, HDAC4, HDAC5, HDAC6, HDAC7, HDAC8, HDAC9, HDAC10, HDAC11, HDAC12, HDAC13, HDAC14, HDAC15, HDAC16, HDAC17, or HDAC18. In some embodiments a selective HDAC2 inhibitor is a compound that inhibits the activity or expression of HDAC2 but does not significantly inhibit the activity or expression of any other HDAC enzymes. In other embodiments a selective HDAC2 inhibitor does not significantly inhibit the activity or expression of any other class I HDAC enzymes. An HDAC1/HDAC2 selective inhibitor is a compound that inhibits the activity or expression of HDAC1 and HDAC2 but does not significantly inhibit the activity or expression of at least one non-class I HDAC enzyme. In some embodiments an HDAC1/HDAC2 selective inhibitor does not significantly inhibit the activity or expression of any non-class I HDAC enzyme. In other embodiments an HDAC1/HDAC2 selective inhibitor does not significantly inhibit the activity or expression of a HDAC3 enzyme. An HDAC1/HDAC2/HDAC3 selective inhibitor is a compound that inhibits the activity or expression of HDAC1 and HDAC2 and HDAC3 but does not significantly inhibit the activity or expression of at least one non-class I HDAC enzyme. In some embodiments an HDAC1/HDAC2/HDAC3 selective inhibitor does not significantly inhibit the activity or expression of any non-class I HDAC enzyme. Significantly inhibit refers to an amount that would detectably alter the activity of the HDAC in a cell such as in vivo. In some embodiments the non-selective HDAC2 inhibitor may be partially selective. For instance, it may act as an inhibitor of one or more other enzymes of HDAC1-HDAC18 but not all. Preferably the HDAC inhibitor does not act as an inhibitor of HDAC1, HDAC5, HDAC6, HDAC7, and HDAC10. In some embodiments, the HDAC2 inhibitor is a selective HDAC1/HDAC2/HDAC10 inhibitor. In some embodiments, the selective HDAC1/HDAC2/HDAC10 inhibitor is BRD-6929. In other embodiments, the HDAC2 inhibitor is a selective HDAC1/HDAC2/HDAC3/HDAC10 inhibitor.

HDAC2 inhibitors include binding peptides such as antibodies, preferably monoclonal antibodies, antibody fragments, scFv, etc that specifically react with the histone deacetylase, small molecule inhibitors (often classically referred to as HDAC inhibitors), and expression inhibitors such as antisense and siRNA.

Studies described in the Examples below were also undertaken to determine which of the 11 histone deacetylases is responsible for the observed function and to identify selective HDAC inhibitors for enhancing memory. It had been discovered that while HDAC1 Tg mice do not show any difference in learning behavior compared to the control mice, HDAC2 Tg mice have impaired learning as evaluated by Pavlovian fear conditioning and Morris water maze tests. Remarkably, HDAC2 neuron specific knockout mice (loss of function) display enhanced learning. Conversely, MS-275, a class 1 HDAC inhibitor (HDAC1/HDAC3 specific), did not facilitate associative learning in mice and MS-275 treated mice showed lower number of c-fos positive cells after fear conditioning training compared to saline treated group. Additional data also demonstrates that HDAC5, HDAC6, HDAC7 and HDAC10 are not useful for enhancing memory. These observations suggest that HDAC2 participates in learning and memory and that it is likely to be the target of inhibition by the general HDAC inhibitors. Even more surprisingly it was discovered that HDAC1/HDAC2 and HDAC1/HDAC2/HDAC3 selective inhibitors were also useful in enhancing learning and memory. Prior studies by some of the instant inventors had demonstrated that HDAC1 activators promote neurogenesis. Thus it was unexpected that HDAC1/HDAC2 inhibitors would be useful for enhancing memory.

HDAC inhibitors include but are not limited to the following compounds, functional analogs and salts thereof: trichostatin A (TSA), trichostatin B, trichostatin C, trapoxin A, trapoxin B, chlamydocin, sodium salts of butyrate, butyric acid, sodium salts of phenylbutyrate, phenylbutyric acid, scriptaid, FR901228, depudecin, oxamflatin, pyroxamide, apicidin B, apicidin C, Helminthsporium carbonum toxin, 2-amino-8-oxo-9,10-epoxy-decanoyl, 3-(4-aroyl-1H-pyrrol-2-yl)-N-hydroxy-2-propenamide, suberoylanilide hydroxamic acid (SAHA), valproic acid, FK228, or m-carboxycinnamic acid bis-hydroxamide. In preferred embodiments the HDAC inhibitor is an HDAC2 inhibitor such as sodium butyrate, SAHA or TSA. Derivatives of the inhibitors showing increased pharmacological half-life are also useful according to the invention (Brettman and Chaturvedi, J. Cli. Pharmacol. 36 (1996), 617-622).

An example of a pan or universal HDAC inhibitor is SAHA. “SAHA” as used herein refers to suberoylanilide hydroxamic acid, analogs, derivatives and polymorphs. Polymorphs of SAHA are described in US Published Patent Application No. 20040122101 which is incorporated by reference.

HDAC2 inhibitors, including HDAC2 selective inhibitors, HDAC1/HDAC2 selective inhibitors and HDAC1/HDAC2/HDAC3 selective inhibitors, of the invention include small molecules as well as inhibitory nucleic acids such as antisense and siRNA. Small molecule HDAC2 inhibitors include for instance compounds of the following formulas:

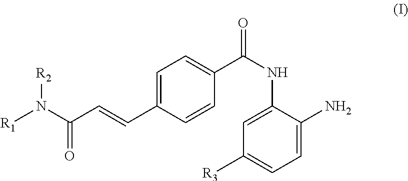

wherein R₁ and R₂ are independently selected from H, substituted or unsubstituted, branched or unbranched, cyclic or acyclic C₁₋₆alkyl, heterocyclyl, C₁₋₆alkylene, heteroaryl, heteroarylene, and heteroarylene-alkylene; and R₃ is aryl or heteroaryl. In some embodiments R₁ is unsubstituted acyclic C₁₋₆alkyl; R₁ is selected from a group consisting of methyl, ethyl, propyl, and butyl; R₁ is heteroarylene-alkylene; R₁ is heteroarylene-C₁₋₆alkylene; R₁ is pyridinyl-ethylene; R₂ is hydrogen; R₃ is heteroaryl; or R₃ is thienyl.

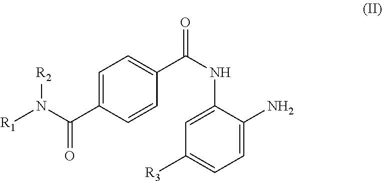

wherein R₁ and R₂ are independently selected from H, substituted or unsubstituted, branched or unbranched, cyclic or acyclic C₁₋₆alkyl, heterocyclyl, C₁₋₆alkylene, heteroaryl, heteroarylene, heteroarylene-alkylene, arylene-alkylene; and heterocyclyl-alkylene optionally substituted; and R₃ is aryl or heteroaryl. In some embodiments R₁ is unsubstituted acyclic C₁₋₆alkyl; R₁ is selected from a group consisting of methyl, ethyl, propyl, and butyl; R₁ is heteroarylene-alkylene; R₁ is heteroarylene-C₁₋₆alkylene; R₁ is pyridinyl-ethylene; R₁ is arylene-alkylene; R₁ is arylene-C₁₋₆alkylene; R₁ is phenyl-ethylene; R₁ is heterocyclyl-alkylene; R₁ is unsubstituted heterocyclyl-C₁₋₆alkylene; R₁ is piperazine-ethylene; R₁ is substituted heterocyclyl-C₁₋₆alkylene; R₁ is substituted piperazine-ethylene; R₁ is C₁₋₆alkylene substituted piperazine-ethylene; R₁ is methyl substituted piperazine-ethylene; R₂ is hydrogen; R₃ is heteroaryl; R₃ is thienyl; or R₃ is pyridinyl.

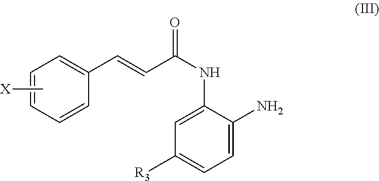

wherein X is —C(O)—N(R₁)(R₂), C₁₋₆alkylene-N(H)—C₁₋₆alkylene-N(R₁)C(O)(R₂); or —N(R₁)C(O)R₂; R₁ and R₂ are independently selected from H, and substituted or unsubstituted, branched or unbranched, cyclic or acyclic C₁₋₆alkyl; and R₃ is alkynyl, aryl, or heteroaryl. In some embodiments X is —C(O)—N(R₁)(R₂); R₁ and R₂ are independently selected from H, unsubstituted, unbranched, acyclic C₁₋₆alkyl; R₁ and R₂ are independently selected from H, methyl, ethyl, propyl, and butyl; R₁ is H; R₁ and R₂ are H; X is —C(O)—NH₂; X is C₁₋₆alkylene-N(R₁)—C₁₋₆alkylene-N(R₁)C(O)(R₂); R₁ is H; X is C₁₋₆alkylene-N(H)—C₁₋₆alkylene-N(H)C(O)(R₂); X is —N(R₁)C(O)R₂; R₁ is H; R₁ is unsubstituted acyclic C₁₋₆alkyl; R₁ is selected from a group consisting of methyl, ethyl, propyl, and butyl; R₂ is unsubstituted acyclic C₁₋₆alky; R₂ is selected from a group consisting of methyl, ethyl, propyl, and butyl; R₃ is heteroaryl; R₃ is thienyl; R₃ is aryl; R₃ is alkynyl; R₃ is C₁₋₆alkynyl; or R₃ is ethynyl.

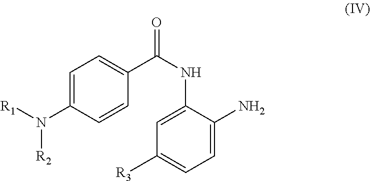

wherein R₁ and R₂ are independently selected from H, and —C(O)—C₁₋₆alkyl; R₃ is optionally substituted aryl, optionally substituted heteroaryl, or aryl-C₁₋₆alkylene. In some embodiments R₁ is H; R₁ and R₂ are H; R₁ is —C(O)—C₁₋₆alkyl; R₁ is —C(O)-methyl; R₁ is —C(O)-methyl and R₂ is H; R₃ is optionally substituted aryl; R₃ is tolyl; R₃ is optionally substituted heteroaryl; R₃ is thienyl; R₃ is aryl-C₁₋₆alkylene; or R₃ is phenyl-ethylene.

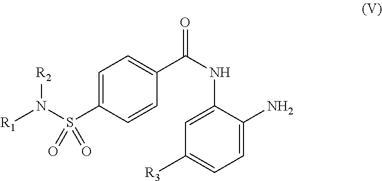

wherein R₁ and R₂ are independently selected from H, and substituted or unsubstituted, branched or unbranched, cyclic or acyclic C₁₋₆alkyl; and R₃ is aryl or heteroaryl. In some embodiments R₁ is H; R₁ and R₂ are H; R₁ is methyl, ethyl, propyl, to or butyl; R₃ is aryl; R₃ is heteroaryl; or R₃ is thienyl.

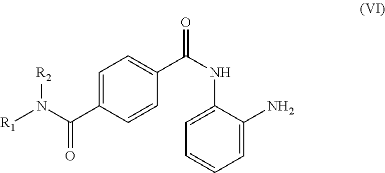

wherein R₁ and R₂ are independently selected from H, substituted or unsubstituted, branched or unbranched, cyclic or acyclic C₁₋₆alkyl, heterocyclyl, heteroaryl, aryl, and aryl-C₁₋₆alkylene. In some embodiments R₁ is H; R₁ and R₂ are H: R₁ is methyl, ethyl, propyl, or butyl; R₁ is aryl-C₁₋₆alkylene; R₁ is phenyl-ethylene; or R₂ is H.

“Alkyl” in general, refers to an aliphatic hydrocarbon group which may be straight, branched or cyclic having from 1 to about 10 carbon atoms in the chain, and all combinations and sub combinations of ranges therein. The term “alkyl” includes both “unsubstituted alkyls” and “substituted alkyls,” the latter of which refers to alkyl moieties having substituents replacing a hydrogen on one or more carbons of the backbone. In preferred embodiments, a straight chain or branched chain alkyl has 12 or fewer carbon atoms in its backbone (e.g., C₁-C₁₂ for straight chain, C₃-C₁₂ for branched chain), and more preferably 6 or fewer, and even more preferably 4 or fewer. Likewise, preferred cycloalkyls have from 3-10 carbon atoms in their ring structure, and more preferably have 5, 6 or 7 carbons in the ring structure. Unless the number of carbons is otherwise specified, “lower alkyl” as used herein means an alkyl group, as defined above, but having from one to ten carbons, more preferably from one to six carbon atoms in its backbone structure, and even more preferably from one to four carbon atoms in its backbone structure. Likewise, “lower alkenyl” and “lower alkynyl” have similar chain lengths. Preferred alkyl groups are lower alkyls. In preferred embodiments, a substituent designated herein as alkyl is a lower alkyl. Alkyl groups include, but are not limited to, methyl, ethyl, n-propyl, isopropyl, n-butyl, isobutyl, t-butyl, n-pentyl, cyclopentyl, isopentyl, neopentyl, n-hexyl, isohexyl, cyclohexyl, cyclooctyl, adamantyl, 3-methylpentyl, 2,2-dimethylbutyl, and 2,3-dimethylbutyl. Alkyl substituents can include, for example, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “alkenyl” refers to unsaturated aliphatic groups analogous in length and possible substitution to the alkyls described above, but that contain at least one double bond.

As used herein, the term “halogen” designates —F, —Cl, —Br or —I; the term “sulfhydryl” means —SH; and the term “hydroxyl” means —OH.

The term “aryl,” alone or in combination, means a carbocyclic aromatic system containing one, two or three rings wherein such rings may be attached together in a pendent manner or may be fused. The term “aryl” embraces aromatic radicals such as phenyl, naphthyl, tetrahydronapthyl, indane and biphenyl, and includes carbocyclic aryl, heterocyclic aryl and biaryl groups, all of which may be optionally substituted. The term “aryl” as used herein includes 5-, 6- and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring can be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocyclyls.

The term “biaryl” represents aryl groups which have 5-14 atoms containing more than one aromatic ring including both fused ring systems and aryl groups substituted with other aryl groups. Such groups may be optionally substituted. Suitable biaryl groups include naphthyl and biphenyl. The term “carbocyclic” refers to a cyclic compounds in which all of the ring members are carbon atoms. Such rings may be optionally substituted. The compound can be a single ring or a biaryl ring. The term “cycloalkyl” embraces radicals having three to ten carbon atoms, such as cyclopropyl, cyclobutyl, cyclopentyl, cyclohexyl, cycloheptyl and norboryl. Such groups may be substituted.

“Heterocyclic” aryl or “heteroaryl” groups are groups which have 5-14 ring atoms wherein 1 to 4 heteroatoms are ring atoms in the aromatic ring and the remainder of the ring atoms being carbon atoms. Suitable heteroatoms include oxygen, sulfur, and nitrogen. Suitable heteroaryl groups include furanyl, thienyl, pyridyl, pyrrolyl, N-lower alkyl pyrrolyl, pyridyl-N-oxide, pyrimidyl, pyrazinyl, imidazolyl, indolyl and the like, all optionally substituted. The term “heterocyclic” refers to cyclic compounds having as ring members atoms of at least two different elements. The compound can be a single ring or a biaryl. Heterocyclic groups include, for example, thiophene, benzothiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

Non-limiting examples of HDAC2 inhibitors useful in the methods of the invention are:

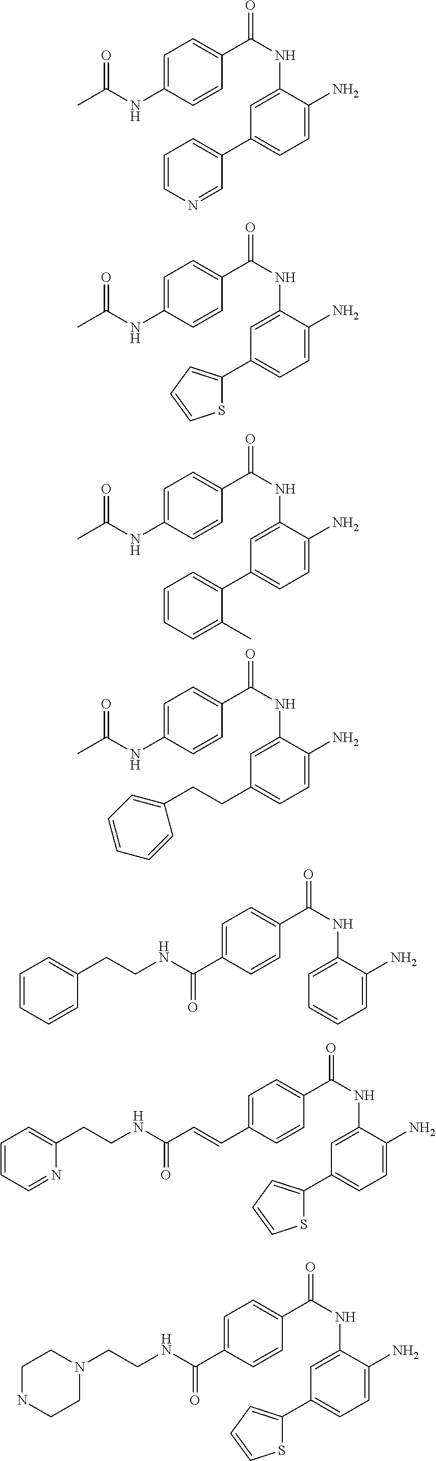

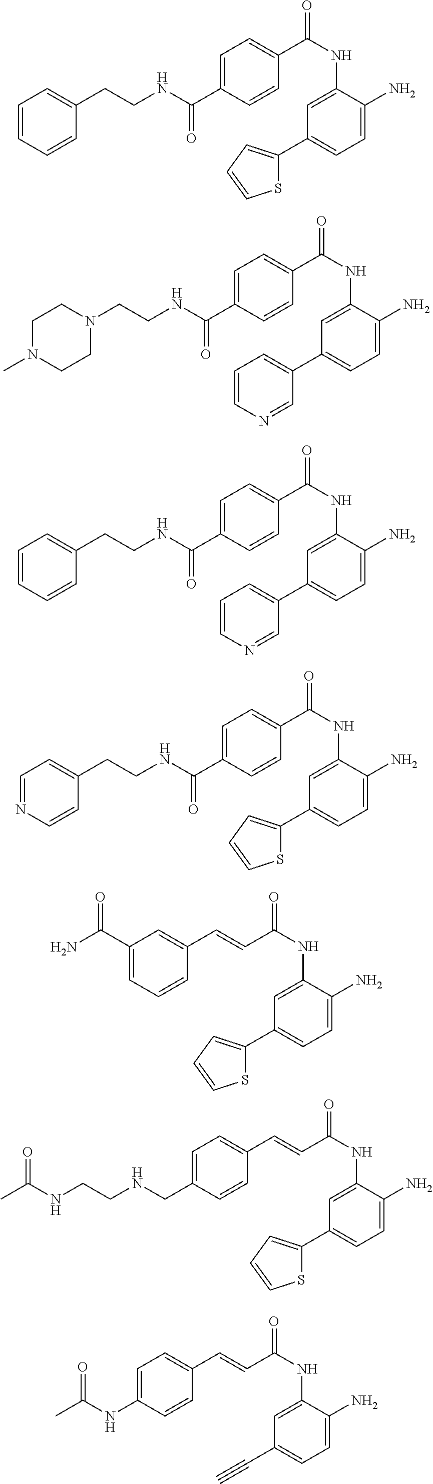

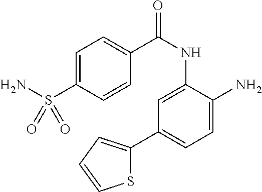

The compounds of the invention may optionally be administered with other compounds such as DNA methylation inhibitors. A DNA methylation inhibitor is an agent that directly or indirectly causes a reduction in the level of methylation of a nucleic acid molecule. DNA methylation inhibitors are well known and routinely utilized in the art and include, but are not limited to, inhibitors of methylating enzymes such as methylases and methyltransferases. Non-limiting examples of DNA methylation inhibitors include 5-azacytidine, 5-aza-2′deoxycytidine (also known as Decitabine in Europe), 5,6-dihydro-5-azacytidine, 5,6-dihydro-5-aza-2′deoxycytidine, 5-fluorocytidine, 5-fluoro-2′deoxycytidine, and short oligonucleotides containing 5-aza-2′deoxycytosine, 5,6-dihydro-5-aza-2′deoxycytosine, and 5-fluoro-2′deoxycytosine, and procainamide, Zebularine, and (−)-egallocatechin-3-gallate.

In addition to the classic small molecule HDAC inhibitors described above, HDAC2 can also be inhibited by nucleic acid based or expression inhibitors such as antisense and RNAi. Thus, the invention embraces inhibitory nucleic acids such as antisense oligonucleotides that selectively bind to nucleic acid molecules encoding HDAC2 to decrease expression and activity of this protein.

As used herein, the term “antisense oligonucleotide” or “antisense” describes an oligonucleotide that is an oligoribonucleotide, oligodeoxyribonucleotide, modified oligoribonucleotide, or modified oligodeoxyribonucleotide which hybridizes under physiological conditions to DNA comprising a particular gene or to an mRNA transcript of that gene and, thereby, inhibits the transcription of that gene and/or the translation of that mRNA. The antisense molecules are designed so as to interfere with transcription or translation of a target gene upon hybridization with the target gene or transcript. Antisense oligonucleotides that selectively bind to a nucleic acid molecule encoding a histone deacetylase are particularly preferred. Those skilled in the art will recognize that the exact length of the antisense oligonucleotide and its degree of complementarity with its target will depend upon the specific target selected, including the sequence of the target and the particular bases which comprise that sequence.

It is preferred that the antisense oligonucleotide be constructed and arranged so as to bind selectively with the target under physiological conditions, i.e., to hybridize substantially more to the target sequence than to any other sequence in the target cell under physiological conditions. Based upon the nucleotide sequences of nucleic acid to molecules encoding histone deacetylase, (e.g., GenBank Accession Nos NP_{—}848512, NP_{—}848510, NP_{—}478057, NP_{—}478056, NP_{—}055522) or upon allelic or homologous genomic and/or cDNA sequences, one of skill in the art can easily choose and synthesize any of a number of appropriate antisense molecules for use in accordance with the present invention. In order to be sufficiently selective and potent for inhibition, such antisense oligonucleotides should comprise at least about 10 and, more preferably, at least about 15 consecutive bases which are complementary to the target, although in certain cases modified oligonucleotides as short as 7 bases in length have been used successfully as antisense oligonucleotides. See Wagner et al., Nat. Med. 1(11):1116-1118, 1995. Most preferably, the antisense oligonucleotides comprise a complementary sequence of 20-30 bases. Although oligonucleotides may be chosen which are antisense to any region of the gene or mRNA transcripts, in preferred embodiments the antisense oligonucleotides correspond to N-terminal or 5′ upstream sites such as translation initiation, transcription initiation or promoter sites. In addition, 3′-untranslated regions may be targeted by antisense oligonucleotides. Targeting to mRNA splicing sites has also been used in the art but may be less preferred if alternative mRNA splicing occurs. In addition, the antisense is targeted, preferably, to sites in which mRNA secondary structure is not expected (see, e.g., Sainio et al., Cell Mol. Neurobiol. 14(5):439-457, 1994) and at which proteins are not expected to bind.

In one set of embodiments, the antisense oligonucleotides of the invention may be composed of “natural” deoxyribonucleotides, ribonucleotides, or any combination thereof. That is, the 5′ end of one native nucleotide and the 3′ end of another native nucleotide may be covalently linked, as in natural systems, via a phosphodiester internucleoside linkage. These oligonucleotides may be prepared by art recognized methods which may be carried out manually or by an automated synthesizer. They also may be produced recombinantly by vectors.

In preferred embodiments, however, the antisense oligonucleotides of the invention also may include “modified” oligonucleotides. That is, the oligonucleotides may be modified in a number of ways which do not prevent them from hybridizing to their target but which enhance their stability or targeting or which otherwise enhance their therapeutic effectiveness.

The term “modified oligonucleotide” as used herein describes an oligonucleotide in which (1) at least two of its nucleotides are covalently linked via a synthetic internucleoside linkage (i.e., a linkage other than a phosphodiester linkage between the 5′ end of one nucleotide and the 3′ end of another nucleotide) and/or (2) a chemical group not normally associated with nucleic acid molecules has been covalently attached to the oligonucleotide. Preferred synthetic internucleoside linkages are phosphorothioates, alkylphosphonates, phosphorodithioates, phosphate esters, alkylphosphonothioates, phosphoramidates, carbamates, carbonates, phosphate triesters, acetamidates, carboxymethyl esters and peptides.

The term “modified oligonucleotide” also encompasses oligonucleotides with a covalently modified base and/or sugar. For example, modified oligonucleotides include oligonucleotides having backbone sugars which are covalently attached to low molecular weight organic groups other than a hydroxyl group at the 3′ position and other than a phosphate group at the 5′ position. Thus modified oligonucleotides may include a 2′-O-alkylated ribose group. In addition, modified oligonucleotides may include sugars such as arabinose instead of ribose.

The present invention, thus, contemplates pharmaceutical preparations containing modified antisense molecules that are complementary to and hybridizable with, under physiological conditions, nucleic acid molecules encoding a histone deacetylase, together with pharmaceutically acceptable carriers. Antisense oligonucleotides may be administered as part of a pharmaceutical composition. In this latter embodiment, it may be preferable that a slow intravenous administration be used. Such a pharmaceutical composition may include the antisense oligonucleotides in combination with any standard physiologically and/or pharmaceutically acceptable carriers which are known in the art. The compositions should be sterile and contain a therapeutically effective amount of the antisense oligonucleotides in a unit of weight or volume suitable for administration to a subject.

The methods of the invention also encompass use of isolated short RNA that directs the sequence-specific degradation of a histone deacetylase mRNA through a process known as RNA interference (RNAi). The process is known to occur in a wide variety of organisms, including embryos of mammals and other vertebrates. It has been demonstrated that dsRNA is processed to RNA segments 21-23 nucleotides (nt) in length, and furthermore, that they mediate RNA interference in the absence of longer dsRNA. Thus, these 21-23 nt fragments are sequence-specific mediators of RNA degradation and are referred to herein as siRNA or RNAi. Methods of the invention encompass the use of these fragments (or recombinantly produced or chemically synthesized oligonucleotides of the same or similar nature) to enable the targeting of histone deacetylase mRNAs for degradation in mammalian cells useful in the therapeutic applications discussed herein.

The nucleotide sequence of HDAC2 is well known in the art and can be used by one of skill in the art using art recognized techniques in combination with the guidance set forth below to produce the appropriate siRNA molecules. Such methods are described in more detail below.

In one embodiment, the invention features a siNA molecule having RNAi activity against target HDAC2 RNA (e.g., coding or non-coding RNA), wherein the siNA molecule comprises a sequence complementary to any HDAC2 RNA sequence, such as that sequence having HDAC2 GenBank Accession No: mRNA NM_{—}001527 for homo sapiens. Chemical modifications can be applied to any siNA construct of the invention. As shown in GenBank Accession No: mRNA NM_{—}001527 the protein sequence of HDAC2 is:

(SEQ ID NO: 1)
MRSPPCGLLRWFGGPLLASWCRCHLRFRAFGTSAGWYRAFPAPP
PLLPPACPSPRDYRPHVSLSPFLSRPSRGGSSSSSSSRRRSPVAAVAGE
PMAYSQGGGKKKVCYYYDGDIGNYYYGQGHPMKPHRIRMTHNLLLNYGL
YRKMEIYRPHKATAEEMTKYHSDEYIKFLRSIRPDNMSEYSKQMQRFNV
GEDCPVFDGLFEFCQLSTGGSVAGAVKLNRQQTDMAVNWAGGLHHAKKS
EASGFCYVNDIVLAILELLKYHQRVLYIDIDIHHGDGVEEAFYTTDRVM
TVSFHKYGEYFPGTGDLRDIGAGKGKYYAVNFPMRDGIDDESYGQIFKP
IISKVMEMYQPSAVVLQCGADSLSGDRLGCFNLTVKGHAKCVEVVKTFN
LPLLMLGGGGYTIRNVARCWTYETAVALDCEIPNELPYNDYFEYFGPDF
KLHISPSNMTNQNTPEYMEKIKQRLFENLRMLPHAPGVQMQAIPEDAVH
EDSGDEDGEDPDKRISIRASDKRIACDEEFSDSEDEGEGGRRNVADHKK
GAKKARIEEDKKETEDKKTDVKEEDKSKDNSGEKTDTKGTKSEQLSNP

The nucleic acid sequence is:

```
(SEQ ID NO: 2)
      1 atgcgctcac ctccctgcgg cctcctgagg tggtttggtg gccccctcct cgcgagttgg
    61 tgccgctgcc acctccgatt ccgagctttc ggcacctctg ccgggtggta ccgagccttc
  121 ccggcgcccc ctcctctcct cccaccggcc tgccattccc cgcgggacta tcgcccccac
  181 gtttccctca gcccttttct ctcccggccg agccgcggcg gcagcagcag cagcagcagc
  241 agcaggagga ggagcccggt ggcggcggtg gccggggagc ccatggcgta cagtcaagga
  301 ggcggcaaaa aaaaagtctg ctactactac gacggtgata ttggaaatta ttattatgga
  361 cagggtcatc ccatgaagcc tcatagaatc cgcatgaccc ataacttgct gttaaattat
  421 ggcttataca gaaaaatgga aatatatagg ccccataaag ccactgccga agaaatgaca
  481 aaatatcaca gtgatgagta tatcaaattt ctacggtcaa taagaccaga taacatgtct
  541 gagtatagta agcagatgca gagatttaat gttggagaag attgtccagt gtttgatgga
  601 ctctttgagt tttgtcagct ctcaactggc ggttcagttg ctggagctgt gaagttaaac
  661 cgacaacaga ctgatatggc tgttaattgg gctggaggat tacatcatgc taagaaatca
  721 gaagcatcag gattctgtta cgttaatgat attgtgcttg ccatccttga attactaaag
  781 tatcatcaga gagtcttata tattgatata gatattcatc atggtgatgg tgttgaagaa
  841 gctttttata caacagatcg tgtaatgacg gtatcattcc ataaatatgg ggaatacttt
  901 cctggcacag gagacttgag ggatattggt gctggaaaag gcaaatacta tgctgtcaat
  961 tttccaatga gagatggtat agatgatgag tcatatgggc agatatttaa gcctattatc
1021 tcaaaggtga tggagatgta tcaacctagt gctgtggtat tacagtgtgg tgcagactca
1081 ttatctggtg atagactggg ttgtttcaat ctaacagtca aaggtcatgc taaatgtgta
1141 gaagttgtaa aaacttttaa cttaccatta ctgatgcttg gaggaggtgg ctacacaatc
1201 cgtaatgttg ctcgatgttg gacatatgag actgcagttg cccttgattg tgagattccc
1261 aatgagttgc catataatga ttactttgag tattttggac cagacttcaa actgcatatt
1321 agtccttcaa acatgacaaa ccagaacact ccagaatata tggaaaagat aaaacagcgt
1381 ttgtttgaaa atttgcgcat gttacctcat gcacctggtg tccagatgca agctattcca
1441 gaagatgctg ttcatgaaga cagtggagat gaagatggag aagatccaga caagagaatt
1501 tctattcgag catcagacaa gcggatagct tgtgatgaag aattctcaga ttctgaggat
1561 gaaggagaag gaggtcgaag aaatgtggct gatcataaga aaggagcaaa gaaagctaga
1621 attgaagaag ataagaaaga aacagaggac aaaaaaacag acgttaagga agaagataaa
1681 tccaaggaca acagtggtga aaaaacagat accaaaggaa ccaaatcaga acagctcagc
1741 aacccctgaa tttgacagtc tcaccaattt cagaaaatca ttaaaaagaa aatattgaaa
1801 ggaaaatgtt ttctttttga agacttctgg cttcatttta tactactttg gcatggactg
1861 tatttatttt caaatggctt tttcgttttt gtttttcttg gcaagtttta ttgtgagttt
1921 ttctaattat gaagcaaaat ttcttttctc caccatgctt tatgtgatag tatttaaaat
1981 tgatgtgagt tattatgtca aaaaaactga tctattaaag aagtaattgg cctttctgag
2041 ctgatttttc catcttttgt aattatcttt attaaaaaat tgtacttgga ttatcttttg
2101 tctgtttatt actacaatat gaagtcttgt ttcagtggct aatgacatca tttctgtaga
2161 cttacaatac actctaggtg aaagataatg attacagctt gaaagataac tatttgctgt
2221 ttctttggga agagtattta tagtaattat tacttatctt tgcaatagaa attctaccac
2281 cttgccctct atagcttagc cagttagtat cagtgaagat taacatccca ttacaattta
2341 tgaaataata cagactctgc aattgagatg taggagttct ttgagttgac ccaaagattc
2401 tcaaaattga aatggaaatt ctgaattgaa agaagaaact gaccagaact tgtattgacc
2461 agacttgcct atagtatatt gctggtttaa aatggaacct gcagacaaaa cctgtttctt
2521 ttactgcatt tacatggcat ccaggttcca ttattattta cgtgacatcc aggtttctaa
2581 ctcaaggaaa taaacacaac tgatttatca ttcagcaact acttattgtg tgtctgccca
2641 ttttaagact gtaggagtgt aactgaatat aatggaaaaa tgctttcact cagagcttac
2701 actcagagct tacattctag tagcaggaag cagacaaatg ggggtaactc ctgctactaa
2761 gatgtgcaaa gaagacaaaa cattttagga acttgccaaa atcagtgaaa tctccctttt
2821 tgtcaggccc acattgattc ttttgagatt aaaaattaca gaatgccaga agataattca
2881 gtcaaaagta tttctcttca gtgcagtaaa atattaaaag aaaaaatatt tctctacaag
2941 cctcttaaat gtttcagaca ttcacaatag cacctagact tttgtaatga acagctgtgc
3001 acccaccccg acttaacaaa tattttactt ttgctatatt tgcttcggtg tttttttctt
3061 aaaatacacc gaaattgaag ctctctttgt gtactttgtc gtttgtaatc ctccctccct
3121 cccagcctta agaagtaatc accattgtga ttattttatc catgtttttg tgaatatttt
3181 acccatgttt ttgtactttt gctacatatt tgttatcaat ctgtaaacct ttatgacatt
3241 aggaactaag aaacttagtc ccttcgttag ggggataatg aaatgtattt agtgtttgtg
3301 aaacatagat ggtatgtatt tggacaattc tgtaactttg ctttttttat ttttattttt
3361 ccatagctta ttggggaaca ggtggtgttt ggttacatga ttaagttctt tagtggtgat
3421 ttgtgggatt ttggtggacc catcacccaa gcagtgtaca ctgcacccta tttgtaatct
3481 tttatccctc gcccccctcc caccatgcct cccgtctacc atgatgatcc tgttttaaat
3541 aagaaaatac catttcgcag gctccagatg ttctggcatc ctccctgtgg atttcccagt
3601 gcctgcagct cacaggacaa caggggctgt ggtagagtca cctatgagat cctggagtag
3661 tggatggagg agatggaaca gtgaagacgg aaactgagct cagtatccgg gtgccaggag
3721 acaaaggccc tttgcttttt ttcatttaat attctgatct acccctgttg acacatgtta
3781 agtatagttc attttgactg ctatgtatta tgttccattg tgtgaacata ctgaaattgt
3841 acacttcaat actatactgg atctccttgg gtgtatttaa gaggttttgt ttttctaagt
3901 agttggttat atacaactaa aacctcaaga gaactatcta aagcaatttc agcaaggtga
3961 tttggtacag cattaataaa cagaaatcag taacacttag tgaccaagtc tgttggaaga
4021 acaaagaccc ccatttgtaa taacaaaatt tttagaaata atatgtaaag aagctatggt
4081 tcttgtgtct agtaaggtca atgtaacata gtaagatgtc agaataccct aatactttaa
4141 aaaattcata taggataaaa atgatatttg aaattggcaa ggaaagacat tattttgtaa
4201 gtggaattgg gacaacaact ggtaaccaaa tggaaaaccc agttttctgc cctccactag
4261 aagatttaaa taggaaaaga taaaactacc aaaaacctaa actcttaaag caaatggatt
4321 gaagaaggcc taagtgtgac accaaactca actataaaag atatatttga taacaaaaaa
4381 aaaattagtt cagtggacca acaaaaactt agaagacaag tcaagaaaaa tgacaaaaga
4441 cagagtggga ggcagatttg taactcatcc aggtcaaaag gctcatatct aaagatagta
4501 gaggaacaaa atgtataagg atgtgaactg ggaaacaaat acatataaat agtttgtaaa
4561 tatgaaaaga tctttaacct cagtaaataa aaagctatag agagacttat tttttaactt
4621 agttttttaa acctattatg tttatttatt ttttcttttt ttgagacgga gtctcgctgt
4681 tgcccaggct ggtgtgcaat ggcgcaatct cggctcactg caacctccgc ctcccaggtt
4741 gaagccattc tcctgcctca gcctcctgag tagctgggat tacaggcgcc tgtcaccacg
4801 cccagctaat tgttcgcatt tttagtagag acggggtttc actatgttgg ccaggctggc
4861 ctcgaactcc tgacctcatg atccacccac cttggccttc caaagtgctg ggattacagg
4921 tgtgagctgc cgcacctggc tgtttatctc ttttttttag agaaaggatc tcggtcaccc
4981 aggatggagt gtggtagcct gatcatatct cactacatct tagaacttct aggcttaggg
5041 tattctccca cctcagcctc ccaagtagct gggactacaa gtgtgcacca tcacacctag
5101 ctgattttta catttttatt ttggagagat ggtgtctccc tgtgttgccc aggctggtca
5161 caaactccta ggctcaagcg attctcctga ctcaggcatg agccaccgta cccggcctaa
5221 acctatcatg ttacagactt agaaagcaac tattgtcaag tgtttgagga aactcaggtc
5281 aggtttggta aactaagata ttaactcaag taaagctctt taattcattt aatgaaggtg
5341 ccacattgtc tcagttctct atggcatggg tgaatgctgt tctaagtcag cattggtact
5401 ctaagctagt tacatcatat ctaagctttg cccttctacc agagctgcta gcattctgtc
5461 aatgggcaat tatttgaagt tcttacattg aagttagtca cctacatctt ctgtttttta
5521 tggttttgat gtagtaatac tgctgaagtt tttttgataa ctgcgattca taatatttgt
5581 tattcatatt gtgatataaa tgataagggc ttttgaaaac aagtagtaat catttcaata
5641 gcttaggatc tccaccataa tcttaggaaa attactaacc tctgtgcctc agttgcttca
5701 tcatttaaaa tgaggaaaat aatagtccct acttaatagg tttgttgtga ggattgagtt
5761 aataacatag ttaatgctca gtaagggtta gctgctattt ttttttcttt tttttttgaa
5821 agagtctcac tctgttgccc aggttggagt gcagtggcat gatcttggct cactgcatcc
5881 tctgcttcct aggttcaggt gattctcatg cttcagcctc ccaagcagct gggagtacag
5941 gtgtgcacca ccacacctgg ctaatttttg tatatttagt agagacgggt tctcaccata
6001 ttgtccaggc tggtcttgaa ctccttacct caaatgatcc acccgcctgg gcctcccaaa
6061 gtgctgggat tacaagcatg agccaccgcg cctggcttgc tattgttatg aggtaaaggt
6121 agatagatgg gtgagagtgg tgccagggga agtgttaaat ttttgagtgt tcctttagat
6181 gccagatggg ttgtatctga gccttttatt gcagtttgat gcctactagt gtgaagacta
6241 ctaggtcata gtggatagag aagcaatctt ttggagacct gattttagca aggatacgaa
6301 taatatttga caactttggg gggatcttga tgcctctgta atttactcaa ggataatctc
6361 aagaaaaatg gcattaagta gattacagaa aaaatagaac tatcatattg ttattattgg
6421 ctatttacat gagcaatgcg gagaaatgtt taggattaca gcatttagaa gcttctcaat
6481 tgctgcattt cctcactgta ccacaagatg gcagatactg catttaaaat ttttttttct
6541 gtgtgttttc tcttatagtc acttggtggc catgtaacaa gcagagcaac atgtattaac
6601 agattctttt tgaatgcaat attggattaa aaactttgaa ttaaaaaaaa aaaaaaaaa
```

Thus the invention features the use of small nucleic acid molecules, referred to as small interfering nucleic acid (siNA) that include, for example: microRNA (miRNA), small interfering RNA (siRNA), double-stranded RNA (dsRNA), and short hairpin RNA (shRNA) molecules. An siNA of the invention can be unmodified or chemically-modified. An siNA of the instant invention can be chemically synthesized, expressed from a vector or enzymatically synthesized as discussed herein. The instant invention also features various chemically-modified synthetic small interfering nucleic acid (siNA) molecules capable of modulating gene expression or activity in cells by RNA interference (RNAi). The use of chemically-modified siNA improves various properties of native siNA molecules through, for example, increased resistance to nuclease degradation in vivo and/or through improved cellular uptake. Furthermore, siNA having multiple chemical modifications may retain its RNAi activity. The siNA molecules of the instant invention provide useful reagents and methods for a variety of therapeutic applications.

Chemically synthesizing nucleic acid molecules with modifications (base, sugar and/or phosphate) that prevent their degradation by serum ribonucleases can increase their potency (see e.g., Eckstein et al., International Publication No. WO 92/07065; Perrault et al, 1990 Nature 344, 565; Pieken et al., 1991, Science 253, 314; Usman and Cedergren, 1992, Trends in Biochem. Sci. 17, 334; Usman et al., International Publication No. WO 93/15187; and Rossi et al., International Publication No. WO 91/03162; Sproat, U.S. Pat. No. 5,334,711; and Burgin et al., supra; all of these describe various chemical modifications that can be made to the base, phosphate and/or sugar moieties of the nucleic acid molecules herein). Modifications which enhance their efficacy in cells, and removal of bases from nucleic acid molecules to shorten oligonucleotide synthesis times and reduce chemical requirements are desired. (All these publications are hereby incorporated by reference herein).

There are several examples in the art describing sugar, base and phosphate modifications that can be introduced into nucleic acid molecules with significant enhancement in their nuclease stability and efficacy. For example, oligonucleotides are modified to enhance stability and/or enhance biological activity by modification with nuclease resistant groups, for example, 2′amino, 2′-C-allyl, 2′-fluoro, 2′-O-methyl, T-H, nucleotide base modifications (for a review see Usman and Cedergren, 1992, TIBS. 17, 34; Usman et al., 1994, Nucleic Acids Symp. Ser. 31, 163; Burgin et al., 1996, Biochemistry, 35, 14090). Sugar modification of nucleic acid molecules have been extensively described in the art (see Eckstein et al., International Publication PCT No. WO 92/07065; Perrault et al. Nature, 1990, 344, 565 568; Pieken et al. Science, 1991, 253, 314317; Usman and Cedergren, Trends in Biochem. Sci., 1992, 17, 334 339; Usman et al. International Publication PCT No. WO 93/15187; Sproat, U.S. Pat. No. 5,334,711 and Beigelman et al., 1995, J. Biol. Chem., 270, 25702; Beigelman et al., International PCT publication No. WO 97/26270; Beigelman et al., U.S. Pat. No. 5,716,824; Usman et al., molecule comprises one or more chemical modifications.

In one embodiment, one of the strands of the double-stranded siNA molecule comprises a nucleotide sequence that is complementary to a nucleotide sequence of a target RNA or a portion thereof, and the second strand of the double-stranded siNA molecule comprises a nucleotide sequence identical to the nucleotide sequence or a portion thereof of the targeted RNA. In another embodiment, one of the strands of the double-stranded siNA molecule comprises a nucleotide sequence that is substantially complementary to a nucleotide sequence of a target RNA or a portion thereof, and the second strand of the double-stranded siNA molecule comprises a nucleotide sequence substantially similar to the nucleotide sequence or a portion thereof of the target RNA. In another embodiment, each strand of the siNA molecule comprises about 19 to about 23 nucleotides, and each strand comprises at least about 19 nucleotides that are complementary to the nucleotides of the other strand.

In some embodiments an siNA is an shRNA, shRNA-mir, or microRNA molecule encoded by and expressed from a genomically integrated transgene or a plasmid-based expression vector. Thus, in some embodiments a molecule capable of inhibiting mRNA expression, or microRNA activity, is a transgene or plasmid-based expression vector that encodes a small-interfering nucleic acid. Such transgenes and expression vectors can employ either polymerase II or polymerase III promoters to drive expression of these shRNAs and result in functional siRNAs in cells. The former polymerase permits the use of classic protein expression strategies, including inducible and tissue-specific expression systems. In some embodiments, transgenes and expression vectors are controlled by tissue specific promoters. In other embodiments transgenes and expression vectors are controlled by inducible promoters, such as tetracycline inducible expression systems.

In some embodiments, a small interfering nucleic acid of the invention is expressed in mammalian cells using a mammalian expression vector. The recombinant mammalian expression vector may be capable of directing expression of the nucleic acid preferentially in a particular cell type (e.g., tissue-specific regulatory elements are used to express the nucleic acid). Tissue specific regulatory elements are known in the art. Non-limiting examples of suitable tissue-specific promoters include the myosin heavy chain promoter, albumin promoter, lymphoid-specific promoters, neuron specific promoters, pancreas specific promoters, and mammary gland specific promoters. Developmentally-regulated promoters are also encompassed, for example the murine hox promoters and the α-fetoprotein promoter.

Other inhibitor molecules that can be used include ribozymes, peptides, DNAzymes, peptide nucleic acids (PNAs), triple helix forming oligonucleotides, antibodies, and aptamers and modified form(s) thereof directed to sequences in gene(s), RNA transcripts, or proteins. Antisense and ribozyme suppression strategies have led to the reversal of a tumor phenotype by reducing expression of a gene product or by cleaving a mutant transcript at the site of the mutation (Carter and Lemoine Br. J. Cancer. 67(5):869-76, 1993; Lange et al., Leukemia. 6(10:1786-94, 1993; Valera et al., J. Biol. Chem. 269(46):28543-6, 1994; Dosaka-Akita et al., Am. J. Clin. Pathol. 102(5):660-4, 1994; Feng et al., Cancer Res. 55(10):2024-8, 1995; Quattrone et al., Cancer Res. 55(1):90-5, 1995; Lewin et al., Nat. Med. 4(8):967-71, 1998). For example, neoplastic reversion was obtained using a ribozyme targeted to an H-Ras mutation in bladder carcinoma cells (Feng et al., Cancer Res. 55(10):2024-8, 1995). Ribozymes have also been proposed as a means of both inhibiting gene expression of a mutant gene and of correcting the mutant by targeted trans-splicing (Sullenger and Cech Nature 371(6498):619-22, 1994; Jones et al., Nat. Med. 2(6):643-8, 1996). Ribozyme activity may be augmented by the use of, for example, non-specific nucleic acid binding proteins or facilitator oligonucleotides (Herschlag et al., Embo J. 13(12):2913-24, 1994; to Jankowsky and Schwenzer Nucleic Acids Res. 24(3):423-9, 1996). Multitarget ribozymes (connected or shotgun) have been suggested as a means of improving efficiency of ribozymes for gene suppression (Ohkawa et al., Nucleic Acids Symp Ser. (29):121-2, 1993).

Triple helix approaches have also been investigated for sequence-specific gene suppression. Triple helix forming oligonucleotides have been found in some cases to bind in a sequence-specific manner (Postel et al., Proc. Natl. Acad. Sci. U.S.A. 88(18):8227-31, 1991; Duval-Valentin et al., Proc. Natl. Acad. Sci. U.S.A. 89(2):504-8, 1992; Hardenbol and Van Dyke Proc. Natl. Acad. Sci. U.S.A. 93(7):2811-6, 1996; Porumb et al., Cancer Res. 56(3):515-22, 1996). Similarly, peptide nucleic acids have been shown to inhibit gene expression (Hanvey et al., Antisense Res. Dev. 1(4):307-17, 1991; Knudsen and Nielson Nucleic Acids Res. 24(3):494-500, 1996; Taylor et al., Arch. Surg. 132(11):1177-83, 1997). Minor-groove binding polyamides can bind in a sequence-specific manner to DNA targets and hence may represent useful small molecules for future suppression at the DNA level (Trauger et al., Chem. Biol. 3(5):369-77, 1996). In addition, suppression has been obtained by interference at the protein level using dominant negative mutant peptides and antibodies (Herskowitz Nature 329(6136):219-22, 1987; Rimsky et al., Nature 341(6241):453-6, 1989; Wright et al., Proc. Natl. Acad. Sci. U.S.A. 86(9):3199-203, 1989). In some cases suppression strategies have led to a reduction in RNA levels without a concomitant reduction in proteins, whereas in others, reductions in RNA have been mirrored by reductions in protein.

The diverse array of suppression strategies that can be employed includes the use of DNA and/or RNA aptamers that can be selected to target, for example HDAC2. Suppression and replacement using aptamers for suppression in conjunction with a modified replacement gene and encoded protein that is refractory or partially refractory to aptamer-based suppression could be used in the invention.

The methods for design of the RNA's that mediate RNAi and the methods for transfection of the RNAs into cells and animals is well known in the art and are readily commercially available (Verma N. K. et al, J. Clin. Pharm. Ther., 28(5):395-404(2004), Mello C. C. et al. Nature, 431(7006)338-42 (2004), Dykxhoorn D. M. et al., Nat. Rev. Mol. Cell. Biol. 4(6):457-67 (2003) Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK)). The RNAs are preferably chemically synthesized using appropriately protected ribonucleoside phosphoramidites and a conventional DNA/RNA synthesizer. Most conveniently, siRNAs are obtained from commercial RNA oligo synthesis suppliers listed herein. In general, RNAs are not too difficult to synthesize and are readily provided in a quality suitable for RNAi. A typical 0.2 μmol-scale RNA synthesis provides about 1 milligram of RNA, which is sufficient for 1000 transfection experiments using a 24-well tissue culture plate format.

The histone deacetylase cDNA specific siRNA is designed preferably by selecting a sequence that is not within 50-100 bp of the start codon and the termination codon, avoids intron regions, avoids stretches of 4 or more bases such as AAAA, CCCC, avoids regions with GC content <30% or >60%, avoids repeats and low complex sequence, and it avoids single nucleotide polymorphism sites. The histone deacetylase siRNA may be designed by a search for a 23-nt sequence motif AA(N19). If no suitable sequence is found, then a 23-nt sequence motif NA(N21) may be used with conversion of the 3′ end of the sense siRNA to TT. Alternatively, the histone deacetylase siRNA can be designed by a search for NAR(N17)YNN. The target sequence may have a GC content of around 50%. The siRNA targeted sequence may be further evaluated using a BLAST homology search to avoid off target effects on other genes or sequences. Negative controls are designed by scrambling targeted siRNA sequences. The control RNA preferably has the same length and nucleotide composition as the siRNA but has at least 4-5 bases mismatched to the siRNA. The RNA molecules of the present invention can comprise a 3′ hydroxyl group. The RNA molecules can be single-stranded or double stranded; such molecules can be blunt ended or comprise overhanging ends (e.g., 5′, 3′) from about 1 to about 6 nucleotides in length (e.g., pyrimidine nucleotides, purine nucleotides). In order to further enhance the stability of the RNA of the present invention, the 3′ overhangs can be stabilized against degradation. The RNA can be stabilized by including purine nucleotides, such as adenosine or guanosine nucleotides. Alternatively, substitution of pyrimidine nucleotides by modified analogues, e.g., substitution of uridine 2 nucleotide 3′ overhangs by 2′-deoxythymidine is tolerated and does not affect the efficiency of RNAi. The absence of a 2′ hydroxyl significantly enhances the nuclease resistance of the overhang in tissue culture medium.

The RNA molecules used in the methods of the present invention can be obtained using a number of techniques known to those of skill in the art. For example, the RNA can be chemically synthesized or recombinantly produced using methods known in the art. Such methods are described in U.S. Published Patent Application Nos. US2002-0086356A1 and US2003-0206884A1 that are hereby incorporated by reference in their entirety.

Any RNA can be used in the methods of the present invention, provided that it has sufficient homology to the HDAC2 gene to mediate RNAi. The RNA for use in the present invention can correspond to the entire HDAC2 gene or a portion thereof. There is no upper limit on the length of the RNA that can be used. For example, the RNA can range from about 21 base pairs (bp) of the gene to the full length of the gene or more. In certain embodiments the preferred length of the RNA of the invention is 21 to 23 nucleotides.

Further, histone deacetylase DNA methylating enzymes can also be inhibited by binding peptides such as antibodies. Numerous histone deacetylase antibodies are commercially available from sources such as Sigma, Vinci Biochem, Cell Signaling Technologies. Such antibodies can be modified to produce antibody fragments or humanized versions. Alternatively therapeutically useful antibodies can be produced using techniques known to those of ordinary skill in the art since HDACs are available.

The therapeutic compounds of the invention may be directly administered to the subject or may be administered in conjunction with a delivery device or vehicle. Delivery vehicles or delivery devices for delivering therapeutic compounds to surfaces have been described. The therapeutic compounds of the invention may be administered alone (e.g., in saline or buffer) or using any delivery vehicles known in the art. For instance the following delivery vehicles have been described: Cochleates; Emulsomes, ISCOMs; Liposomes; Live bacterial vectors (e.g., Salmonella, Escherichia coli, Bacillus calmatte-guerin, Shigella, Lactobacillus); Live viral vectors (e.g., Vaccinia, adenovirus, Herpes Simplex); Microspheres; Nucleic acid vaccines; Polymers; Polymer rings; Proteosomes; Sodium Fluoride; Transgenic plants; Virosomes; Virus-like particles. Other delivery vehicles are known in the art and some additional examples are provided below.

The term effective amount of a therapeutic compound of the invention refers to the amount necessary or sufficient to realize a desired biologic effect. For example, as discussed above, an effective amount of a therapeutic compounds of the invention is that amount sufficient to re-establish access to a memory. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject. The effective amount for any particular application can vary depending on such factors as the disease or condition being treated, the particular therapeutic compounds being administered the size of the subject, or the severity of the disease or condition. One of ordinary skill in the art can empirically determine the effective amount of a particular therapeutic compounds of the invention without necessitating undue experimentation. Compositions of the invention include compounds as described herein, or a pharmaceutically acceptable salt or hydrate thereof.

Subject doses of the compounds described herein for delivery typically range from about 0.1 μg to 10 mg per administration, which depending on the application could be given daily, weekly, or monthly and any other amount of time therebetween. The doses for these purposes may range from about 10 μg to 5 mg per administration, and most typically from about 100 μg to 1 mg, with 2-4 administrations being spaced days or weeks apart. In some embodiments, however, parenteral doses for these purposes may be used in a range of 5 to 10,000 times higher than the typical doses described above.

In one embodiment, the composition is administered once daily at a dose of about 200-600 mg. In another embodiment, the composition is administered twice daily at a dose of about 200-400 mg. In another embodiment, the composition is administered twice daily at a dose of about 200-400 mg intermittently, for example three, four, or five days per week. In another embodiment, the composition is administered three times daily at a dose of about 100-250 mg. In one embodiment, the daily dose is 200 mg, which can be administered once-daily, twice-daily, or three-times daily. In one embodiment, the daily dose is 300 mg, which can be administered once-daily or twice-daily. In one embodiment, the daily dose is 400 mg, which can be administered once-daily or twice-daily. The HDAC inhibitor can be administered in a total daily dose of up to 800 mg once, twice or three times daily, continuously (i.e., every day) or intermittently (e.g., 3-5 days a week).

For any compound described herein the therapeutically effective amount can be initially determined from animal models. A therapeutically effective dose can also be determined from human data for HDAC inhibitors which have been tested in humans (e.g. for the treatment of cancer) and for compounds which are known to exhibit similar pharmacological activities. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

Toxicity and efficacy of the prophylactic and/or therapeutic protocols of the present invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Prophylactic and/or therapeutic agents that exhibit large therapeutic indices are preferred. While prophylactic and/or therapeutic agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of the prophylactic and/or therapeutic agents for use in humans. The dosage of such agents lies preferably within a range of circulating concentrations that include the ED₅₀ with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein.

Multiple doses of the molecules of the invention are also contemplated. In some instances, when the molecules of the invention are administered with another therapeutic a sub-therapeutic dosage of either agent, or a sub-therapeutic dosage of both, is used. A “sub-therapeutic dose” as used herein refers to a dosage which is less than that dosage which would produce a therapeutic result in the subject if administered in the absence of the other agent. Thus, the sub-therapeutic dose of, for instance, an anti-Alzheimer's agent is one which would not produce the desired therapeutic result in the subject in the absence of the administration of the compounds of the invention.

The formulations of the invention are administered in pharmaceutically acceptable solutions, which may routinely contain pharmaceutically acceptable concentrations of salt, buffering agents, preservatives, compatible carriers, and optionally other therapeutic ingredients.

For use in therapy, an effective amount of the therapeutic compounds of the invention can be administered to a subject by any mode that delivers the therapeutic agent or compound to the desired surface, e.g., mucosal, systemic. Administering the pharmaceutical composition of the present invention may be accomplished by any means known to the skilled artisan. Preferred routes of administration include but are not limited to oral, parenteral, intramuscular, intranasal, sublingual, intratracheal, inhalation, ocular, vaginal, rectal and intracerebroventricular.

For oral administration, the therapeutic compounds of the invention can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, i.e. EDTA for neutralizing internal acid conditions or may be administered without any carriers.

Also specifically contemplated are oral dosage forms of the above component or components. The component or components may be chemically modified so that oral delivery of the derivative is efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, 1981, “Soluble Polymer-Enzyme Adducts” In: Enzymes as Drugs, Hocenberg and Roberts, eds., Wiley-Interscience, New York, N.Y., pp. 367-383; Newmark, et al., 1982, J. Appl. Biochem. 4:185-189. Other polymers that could be used are poly-1,3-dioxolane and poly-1,3,6-tioxocane. Preferred for pharmaceutical usage, as indicated above, are polyethylene glycol moieties.

The location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the therapeutic agent or by release of the biologically active material beyond the stomach environment, such as in the intestine.

To ensure full gastric resistance a coating impermeable to at least pH 5.0 is important. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic i.e. powder; for liquid forms, a soft gelatin shell may be used.

The shell material of cachets could be thick starch or other edible paper. For pills, lozenges, molded tablets or tablet triturates, moist massing techniques can be used.

The therapeutic can be included in the formulation as fine multi-particulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the therapeutic agent may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, α-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential non-ionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the therapeutic agent either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

Also contemplated herein is pulmonary delivery of the therapeutic compounds of the invention. The therapeutic agent is delivered to the lungs of a mammal while inhaling and traverses across the lung epithelial lining to the blood stream. Other reports of inhaled molecules include Adjei et al., 1990, Pharmaceutical Research, 7:565-569; Adjei et al., 1990, International Journal of Pharmaceutics, 63:135-144 (leuprolide acetate); Braquet et al., 1989, Journal of Cardiovascular Pharmacology, 13(suppl. 5):143-146 (endothelin-1); Hubbard et al., 1989, Annals of Internal Medicine, Vol. III, pp. 206-212 (a1-antitrypsin); Smith et al., 1989, J. Clin. Invest. 84:1145-1146 (a-1-proteinase); Oswein et al., 1990, “Aerosolization of Proteins”, Proceedings of Symposium on Respiratory Drug Delivery II, Keystone, Colorado, March, (recombinant human growth hormone); Debs et al., 1988, J. Immunol. 140:3482-3488 (interferon-g and tumor necrosis factor alpha) and Platz et al., U.S. Pat. No. 5,284,656 (granulocyte colony stimulating factor). A method and composition for pulmonary delivery of drugs for systemic effect is described in U.S. Pat. No. 5,451,569, issued Sep. 19, 1995 to Wong et al.

Contemplated for use in the practice of this invention are a wide range of mechanical devices designed for pulmonary delivery of therapeutic products, including but to not limited to nebulizers, metered dose inhalers, and powder inhalers, all of which are familiar to those skilled in the art.

Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colorado; the Ventolin metered dose inhaler, manufactured by Glaxo Inc., Research Triangle Park, N.C.; and the Spinhaler powder inhaler, manufactured by Fisons Corp., Bedford, Mass.

All such devices require the use of formulations suitable for the dispensing of therapeutic agent. Typically, each formulation is specific to the type of device employed and may involve the use of an appropriate propellant material, in addition to the usual diluents, and/or carriers useful in therapy. Also, the use of liposomes, microcapsules or microspheres, inclusion complexes, or other types of carriers is contemplated. Chemically modified therapeutic agent may also be prepared in different formulations depending on the type of chemical modification or the type of device employed.

Formulations suitable for use with a nebulizer, either jet or ultrasonic, will typically comprise therapeutic agent dissolved in water at a concentration of about 0.1 to 25 mg of biologically active compound per mL of solution. The formulation may also include a buffer and a simple sugar (e.g., for stabilization and regulation of osmotic pressure). The nebulizer formulation may also contain a surfactant, to reduce or prevent surface induced aggregation of the compound caused by atomization of the solution in forming the aerosol.

Formulations for use with a metered-dose inhaler device will generally comprise a finely divided powder containing the therapeutic agent suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing therapeutic agent and may also include a bulking agent, such as lactose, sorbitol, sucrose, or mannitol in amounts which facilitate dispersal of the powder from the device, e.g., 50 to 90% by weight of the formulation. The therapeutic agent should most advantageously be prepared in particulate form with an average particle size of less than 10 mm (or microns), most preferably 0.5 to 5 mm, for most effective delivery to the distal lung.

Nasal delivery of a pharmaceutical composition of the present invention is also contemplated. Nasal delivery allows the passage of a pharmaceutical composition of the present invention to the blood stream directly after administering the therapeutic product to the nose, without the necessity for deposition of the product in the lung. Formulations for nasal delivery include those with dextran or cyclodextran.

For nasal administration, a useful device is a small, hard bottle to which a metered dose sprayer is attached. In one embodiment, the metered dose is delivered by drawing the pharmaceutical composition of the present invention solution into a chamber of defined volume, which chamber has an aperture dimensioned to aerosolize and aerosol formulation by forming a spray when a liquid in the chamber is compressed. The chamber is compressed to administer the pharmaceutical composition of the present invention. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the drug.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science 249:1527-1533, 1990, which is incorporated herein by reference.

The therapeutic compounds of the invention and optionally other therapeutics may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The pharmaceutical compositions of the invention contain an effective amount of a therapeutic compound of the invention optionally included in a pharmaceutically-acceptable carrier. The term pharmaceutically-acceptable carrier means one or more compatible solid or liquid filler, diluents or encapsulating substances which are suitable for administration to a human or other vertebrate animal. The term carrier denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

The therapeutic agents may be delivered to the brain using a formulation capable of delivering a therapeutic agent across the blood brain barrier. One obstacle to delivering therapeutics to the brain is the physiology and structure of the brain. The blood-brain barrier is made up of specialized capillaries lined with a single layer of endothelial cells. The region between cells is sealed with a tight junction, so the only access to the brain from the blood is through the endothelial cells. The barrier allows only certain substances, such as lipophilic molecules through and keeps other harmful compounds and pathogens out. Thus, lipophilic carriers are useful for delivering non-lipohilic compounds to the brain. For instance, DHA, a fatty acid naturally occurring in the human brain has been found to be useful for delivering drugs covalently attached thereto to the brain (Such as those described in U.S. Pat. No. 6,407,137). U.S. Pat. No. 5,525,727 describes a dihydropyridine pyridinium salt carrier redox system for the specific and sustained delivery of drug species to the brain. U.S. Pat. No. 5,618,803 describes targeted drug delivery with phosphonate derivatives. U.S. Pat. No. 7,119,074 describes amphiphilic prodrugs of a therapeutic compound conjugated to an PEG-oligomer/polymer for delivering the compound across the blood brain barrier. The compounds described herein may be modified by covalent attachment to a lipophilic carrier or co-formulation with a lipophilic carrier. Others are known to those of skill in the art.

The therapeutic agents of the invention may be delivered with other therapeutics for enhancing memory retrieval or treating other symptoms or causes of disorders associated with the memory loss. For instance, environmental enrichment (EE) has been used for enhancing memories. EE involves creating a stimulating environment around a subject. Other therapeutics may also be combined to treat the underlying disorder or to enhance memory recall.

Examples of combinations of the compounds of the present invention with other drugs in either unit dose or kit form include combinations with: anti-Alzheimer's agents, beta-secretase inhibitors, gamma-secretase inhibitors, HMG-CoA reductase inhibitors, NSAID's including ibuprofen, N-methyl-D-aspartate (NMDA) receptor antagonists, such as memantine, cholinesterase inhibitors such as galantamine, rivastigmine, donepezil, and tacrine, vitamin E, CB-1 receptor antagonists or CB-1 receptor inverse agonists, antibiotics such as doxycycline and rifampin, anti-amyloid antibodies, or other drugs that affect receptors or enzymes that either increase the efficacy, safety, convenience, or reduce unwanted side effects or toxicity of the compounds of the present invention. The compounds of the invention may also be delivered in a cocktail of multiple HDAC inhibitors. The foregoing list of combinations is illustrative only and not intended to be limiting in any way.

The invention also includes articles, which refers to any one or collection of components. In some embodiments the articles are kits. The articles include pharmaceutical or diagnostic grade compounds of the invention in one or more containers. The article may include instructions or labels promoting or describing the use of the compounds of the invention.

As used herein, “promoted” includes all methods of doing business including methods of education, hospital and other clinical instruction, pharmaceutical industry activity including pharmaceutical sales, and any advertising or other promotional activity including written, oral and electronic communication of any form, associated with compositions of the invention in connection with treatment of cognitive disorders such as Alzheimer's disease.

“Instructions” can define a component of promotion, and typically involve written instructions on or associated with packaging of compositions of the invention. Instructions also can include any oral or electronic instructions provided in any manner.

Thus the agents described herein may, in some embodiments, be assembled into pharmaceutical or diagnostic or research kits to facilitate their use in therapeutic, diagnostic or research applications. A kit may include one or more containers housing the components of the invention and instructions for use. Specifically, such kits may include one or more agents described herein, along with instructions describing the intended therapeutic application and the proper administration of these agents. In certain embodiments agents in a kit may be in a pharmaceutical formulation and dosage suitable for a particular application and for a method of administration of the agents.

The kit may be designed to facilitate use of the methods described herein by physicians and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the invention. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which instructions can also reflects approval by the agency of manufacture, use or sale for human administration.

The kit may contain any one or more of the components described herein in one or more containers. As an example, in one embodiment, the kit may include instructions for mixing one or more components of the kit and/or isolating and mixing a sample and applying to a subject. The kit may include a container housing agents described herein. The agents may be prepared sterilely, packaged in syringe and shipped refrigerated. Alternatively it may be housed in a vial or other container for storage. A second container may have other agents prepared sterilely. Alternatively the kit may include the active agents premixed and shipped in a syringe, vial, tube, or other container.

The kit may have a variety of forms, such as a blister pouch, a shrink wrapped pouch, a vacuum sealable pouch, a sealable thermoformed tray, or a similar pouch or tray form, with the accessories loosely packed within the pouch, one or more tubes, containers, a box or a bag. The kit may be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kit may also include other components, depending on the specific application, for example, containers, cell media, salts, buffers, reagents, syringes, needles, a fabric, such as gauze, for applying or removing a disinfecting agent, disposable gloves, a support for the agents prior to administration etc.

The compositions of the kit may be provided as any suitable form, for example, as liquid solutions or as dried powders. When the composition provided is a dry powder, the powder may be reconstituted by the addition of a suitable solvent, which may also be provided. In embodiments where liquid forms of the composition are sued, the liquid form may be concentrated or ready to use. The solvent will depend on the compound and the mode of use or administration. Suitable solvents for drug compositions are well known and are available in the literature. The solvent will depend on the compound and the mode of use or administration.

The kits, in one set of embodiments, may comprise a carrier means being compartmentalized to receive in close confinement one or more container means such as vials, tubes, and the like, each of the container means comprising one of the separate elements to be used in the method. For example, one of the containers may comprise a positive control for an assay. Additionally, the kit may include containers for other components, for example, buffers useful in the assay.

The present invention also encompasses a finished packaged and labeled pharmaceutical product. This article of manufacture includes the appropriate unit dosage form in an appropriate vessel or container such as a glass vial or other container that is hermetically sealed. In the case of dosage forms suitable for parenteral administration the active ingredient is sterile and suitable for administration as a particulate free solution. In other words, the invention encompasses both parenteral solutions and lyophilized powders, each being sterile, and the latter being suitable for reconstitution prior to injection. Alternatively, the unit dosage form may be a solid suitable for oral, transdermal, topical or mucosal delivery.

In a preferred embodiment, the unit dosage form is suitable for intravenous, intramuscular or subcutaneous delivery. Thus, the invention encompasses solutions, preferably sterile, suitable for each delivery route.

In another preferred embodiment, compositions of the invention are stored in containers with biocompatible detergents, including but not limited to, lecithin, taurocholic acid, and cholesterol; or with other proteins, including but not limited to, gamma globulins and serum albumins. More preferably, compositions of the invention are stored with human serum albumins for human uses, and stored with bovine serum albumins for veterinary uses.

As with any pharmaceutical product, the packaging material and container are designed to protect the stability of the product during storage and shipment. Further, the products of the invention include instructions for use or other informational material that advise the physician, technician or patient on how to appropriately prevent or treat the disease or disorder in question. In other words, the article of manufacture includes instruction means indicating or suggesting a dosing regimen including, but not limited to, actual doses, monitoring procedures and other monitoring information.

More specifically, the invention provides an article of manufacture comprising packaging material, such as a box, bottle, tube, vial, container, sprayer, insufflator, intravenous (i.v.) bag, envelope and the like; and at least one unit dosage form of a pharmaceutical agent contained within said packaging material. The invention also provides an article of manufacture comprising packaging material, such as a box, bottle, tube, vial, container, sprayer, insufflator, intravenous (i.v.) bag, envelope and the like; and at least one unit dosage form of each pharmaceutical agent contained within said packaging material. The invention further provides an article of manufacture comprising packaging material, such as a box, bottle, tube, vial, container, sprayer, insufflator, intravenous (i.v.) bag, envelope and the like; and at least one unit dosage form of each pharmaceutical agent contained within said packaging material. The invention further provides an article of manufacture comprising a needle or syringe, preferably packaged in sterile form, for injection of the formulation, and/or a packaged alcohol pad.

In a specific embodiment, an article of manufacture comprises packaging material and a pharmaceutical agent and instructions contained within said packaging material, wherein said pharmaceutical agent is a HDAC2 inhibitor and a pharmaceutically acceptable carrier, and said instructions indicate a dosing regimen for preventing, treating or managing a subject with cognitive disorders such as Alzheimer's disease.

Therapeutic Monitoring: The adequacy of the treatment parameters chosen, e.g. dose, schedule, adjuvant choice and the like, is determined by conventional methods for monitoring memory. In addition, the clinical condition of the patient can be monitored for the desired effect, e.g. increases in cognitive function. If inadequate effect is achieved then the patient can be boosted with further treatment and the treatment parameters can be modified, such as by increasing the amount of the composition of the invention and/or other active agent, or varying the route of administration.

The present invention is further illustrated by the following Examples, which in no way should be construed as further limiting. The Examples, data and Figures of U.S. patent application Ser. No. 11/998,834 as well as U.S. Provisional Patent Application 61/119,698, both of overlapping inventorship are hereby incorporated by reference.

### EXAMPLES

### Methods

Environmental enrichment: Up to four mice were continuously housed in a cage that contained two wheels for voluntary running and a variety of toys (obtained form from Petco) to create tunnels, and climbing devices. Food and water was ad libitum. The food was hidden within the bedding. Toys and running wheels were changed on a daily basis.

Cannulation and injection: Microcannula were inserted into the lateral brain ventricles. Sodiumbutyrate (Sigma; St. Louis, Mo.) was dissolved in artificial cerebrospinal fluid (aCSF). A stock solution of TSA (Sigma) was dissolved in DMSO and diluted with aCSF before injection.

Generation of HDAC overexpression animals The mouse HDAC1 or HDAC2 coding sequence was placed into exon 1 of the Tau gene, in-frame with the endogenous initiation codon, thereby creating a fusion protein that contains the first 31 amino acids of Tau. HDAC2 KO was produced in the laboratory of R.A.D. and engineered to contain loxP recombination sites such that Cre-mediated recombination deletes exons 5 and 6 which encodes the key catalytic core of the HDAC protein.

Chemical delivery Sodium butyrate (sigma) was dissolved in saline. HDAC inhibitors were dissolved in DMSO in 50 mg/ml and diluted with saline immediate before injection (100 ul-150 ul, i.p.).

Immunoblotting and staining Lysates for immunoblotting were prepared as described herein (see also Fischer, A. et al. Recovery of learning and memory is associated with chromatin remodeling. Nature 447 (7141), 178-182 (2007).). Briefly, to isolate histones, brain tissue was homogenized in TX-buffer (50 mM Tris HCl, 150 mM NACl, 2 mM EDTA, 1% Triton-100) and incubated at 4° C. for 15 min before centrifugation at 2,000 r.p.m. (400 g) for 10 min. After a wash-step in TX-buffer the pellet was dissolved in TX-buffer containing 0.2 M HCl and incubated on ice for 30 min, before a second centrifugation at 10,000 r.p.m. (9,300 g) for 10 min. The supernatant was used for immunoblotting. Immunoblot data were quantified by measuring the band intensity using NIH imaging software and UN-SCAN-it gel digitizing software (Silk Scientific). Immunostaining was performed as described herein (see also Fischer, A. et al. Recovery of learning and memory is associated with chromatin remodeling. Nature 447 (7141), 178-182 (2007).) using LSMetal0 software and a confocal microscope (Zeiss).

Gene targeting construct for HDAC1 overexpression (OE) mice. The ˜1200 nt-long mouse HDAC1 cDNA was amplified from a brain cDNA library and confirmed by sequencing. The cDNA was then cloned upstream of the polyadenylation (pA) signal of pC8N2 with a SpeI blunt ligation, subsequently HDAC1-pA was cloned into pBSK (Stratagene). A pGKneoLoxP sequence was directionally inserted into the XhoI-Kpn1 site downstream of the HDAC1-pA in pBSK. The HDAC1-pA-neo was released with XmaI-Acc65 and cloned in frame into exon 1 of the Tau gene. The Tau targeting arms were taken from pTauKR and modified by insertion of a XmaI and BsiWI linker in the unique NcoI site. The resulting targeting vector (pTH1) containing the in frame fusion of HDAC1 coding sequence with exon 1 of Tau was confirmed by sequencing. 3-6-month-old mice were used for the behavior test and further analysis.

Gene targeting construct for HDAC2 overexpression (OE) mice The mouse HDAC2 cDNA was obtained using RT PCR from mouse brain tissue. It was sequenced and subcloned into the XhoI-EcoR1 site of the Topo-TA vector (Invitrogen). The pTH1 targeting vector (described above) was cut open with SmaI-SalI to release HDAC1. The HDAC2 cDNA was cut out from Topo-TA with an EcoRI-XhoI and cloned into the SmaI-SalI site of pTH1, to create the pTH2 targeting vector. The in frame fusion of HDAC2 to exon 1 of Tau was verified by sequencing of pTH2.

The targeting vectors pTH1 and pTH2 were linearized with SacI and electroporated into V6.5 (129XC57BL/6) F1 embryonic stem (ES) cell line. We picked 96 neomycin resistant clones, of which 46 were analyzed by southern blots. We only used a 3′ external probe, after digestion with BamHI (Left) and EcoRI (Right). Wild-type clones display a 8.8-kb band. The correct targeting event results in a band-shift to 13 kb for the targeted allele. 5 clones were correctly targeted. Two clones were used to generate chimeras by injections into (DBA/2XC57BL/6) F1 blastocysts. Chimeras were mated to C57BL/6 females and offspring was analyzed for germline transmission. The heterozygous knock-in strains were maintained in a mixed background and were mated to obtain homozygous animals. 3-6-month-old mice were used for the behavior test and further analysis.

Generation of Hdac2 KO mice The Hdac2 floxed allele was generated by flanking exon 5 and exon 6 with loxP recombination sites, assuring the deletion of the HDAC-catalytic core of the protein after Cre-recombinase mediated deletion. Upon successful targeting of ES-cells and subsequent derivation of chimeric mice, we established a mouse strain carrying a foxed allele of Hdac2 (Hdac2^{L})(FVB). Infection of mouse embryonic fibroblasts with retroviruses expressing Cre-recombinase resulted in complete ablation of Hdac2 only in MEFs carrying two Hdac2 floxed alleles. This indicates that the floxed Hdac2 allele is functional and results in an Hdac2 null-genotype upon Cre-recombinase expression. Deletion of Hdac2 in the germline using EIIa-Cre or Nestin-Cre transgenic mice resulted in viable and fertile Hdac2^{-/-} mice with no obvious histological abnormalities up to a year of age. Crossing Hdac2^{+/-} mice gave rise to viable Hdac2-deficient mice, but these mice were born with a 2-fold lower frequency than expected from a normal Mendelian ratio (9 Hdac2^{-/-} mice out of 79 littermates, versus 20 out of 79 expected. Although Hdac2^{-/-} mice are viable and are capable of producing offspring their fertility is compromised (data not shown). Hdac2^{-/-} mice (males and females) were approximately 25% smaller compared to wild-type and heterozygote littermates (data not shown). The animals used for behavior tests are in FVBxC57/BL6 background and mated to each other to obtain homozygous animals. 3-6-month-old mice were used for the behavior test and further analysis. There was no difference in behavior tests between males and females.

Fear conditioning tests Context-dependent fear conditioning. Training consists of a 3 min exposure of mice to the conditioning box (context) followed by a foot shock (2 sec, 0.5/0.8/1.0 mA, constant current). The memory test was performed 24 hr later by re-exposing the mice for 3 min into the conditioning context. Freezing, defined as a lack of movement except for heart beat and respiration associated with a crouching posture, was recorded every 10 sec by two trained observers (one was unaware of the experimental conditions) during 3 min (a total of 18 sampling intervals). The number of observations indicating freezing obtained as a mean from both observers was expressed as a percentage of the total number of observations.

For short time memory test, the memory test was performed 3 hrs after the foot shock training.

Tone-dependent fear conditioning. Training consisted of a 3 min exposure of mice to the conditioning box (context), followed by a tone [30 sec, 20 kHz, 75 dB sound pressure level (SPL)] and a foot shock (2 sec, 0.8 mA, constant current). The memory test was performed 24 hr later by exposing the mice for 3 min to a novel context followed by an additional 3 min exposure to a tone (10 kHz, 75 dB SPL). Freezing was recorded every 10 sec by two nonbiased observers as described above.

Morris water maze test The water maze paradigm was performed in a circular tank (diameter 1.8 m) filled with opaque water. A platform (11×11 cm) was submerged below the water's surface in the center of the target quadrant. The swimming path of the mice was recorded by a video camera and analyzed by the Videomot 2 software (TSE). For each training session, the mice were placed into the maze consecutively from four random points of the tank. Mice were allowed to search for the platform for 60 s. If the mice did not find the platform within 60 s, they were gently guided to it. Mice were allowed to remain on the platform for 15 s. Two training trials were given every day; the latency for each trial was recorded for analysis. During the memory test (probe test), the platform was removed from the tank, and the mice were allowed to swim in the maze for 60 s.

Spatial working memory on elevated T-maze Mice were maintained on a restricted feeding schedule at 85% of their free-feeding weight. Spatial working memory was first assessed on an elevated plastic T-maze. This consisted of a start arm (47×10 cm) and two identical goal arms (35×10 cm), surrounded by a 10 cm high wall. A plastic food well was located 3 cm from the end of each goal arm. The maze was located 1 m above the floor in a well lit laboratory that contained various prominent distal extramaze cues. The mice were habituated to the maze, and to drinking sweetened, condensed milk, over several days before spatial non-matching-to-place testing.

Each trial consisted of a sample run and a choice run. On the sample run, the mice were forced either left or right by the presence of a plastic block, according to a pseudorandom sequence (with equal numbers of left and right turns per session, and with no more than two consecutive turns in the same direction). A reward consisting of 0.07 ml of sweetened, condensed milk (diluted 50/50 with water) was available in the food well at the end of the arm. The block was then removed, and the mouse was placed, facing the experimenter, at the end of the start arm and allowed a free choice of either arm. The time interval between the sample run and the choice run was approximately 15 s. The animal was rewarded for choosing the previously unvisited arm (that is, for alternating). Mice were run one trial at a time with an inter-trial interval (ITI) of approximately 10 min. Each daily session consisted of 4 trials, and mice received 24 trials in total.

Chemical administration Suberoylanilide hydroxamic acid (SAHA) was synthesized as described previously in WO 93/07148 PTC/US92/08454. Sodium butyrate was purchased from Sigma (cat.B5887). SAHA and WT-161 were dissolved in DMSO as stock solutions and diluted in saline just before injection. Sodium butyrate was prepared in saline. Mice received intraperitoneal injection daily with either SAHA or saline for 10 days or 21 days.

Golgi impregnation Golgi-Cox-stained brains were cut to 200 μm thick cross-sections with vibratome and analyzed using a Zeiss 200 Axiovert microscope and Openlab software. The number of apical and basal spines on hippocampal CA1 pyramidal neurons was counted blind to the genotype. For each experimental group, a minimum of 10 cells per slice (animal number n=3) were analyzed. CA1 hippocampal neurons within the region −1.4 mm to −1.6 mm (relative to the bregma position) were included for the analysis.

Virus mediated spine labeling. Tomato expressing HSV (0.5 μl, gift from Rachael Neve) was stereo-injected into both sides of area CA 1 or dentate gyrus with 0.05 μl/min rate. Mice were sacrificed 48 hrs after injection. Brains were fixed with 4% PFA and sectioned with vibratome (50 μm, Leica). Hippocampal slices were scanned with a confocal microscope. Obtained image stacks were reconstructed and analyzed using image J.

Immunohistochemistry Immunohistochemical analysis was performed as described before (Guan, J. S., et al., Cell, 2005. 122(4): p. 619-31.). Antibodies were used in a 1:1000 concentration. Anti-HDAC1, and anti-HDAC2 antibodies were purchased from Abcam. Anti-Ac-lysine, anti-Ac-H4K5, anit-Ac-H4K12, anti-Ac-H3K16, anti-CREB, anti-AKT and anti-CaMKIIa antibodies were purchased from Cell Signaling. Anti-Ac-α-tubulin (K40), anti-actin and anti-synaptophysin (SVP-38) antibodies were purchased from Sigma. Anti-NR2A and anti-NR2B were purchased from BD Biosciences. Anti-β-catenin, anti-EGR1, anti-c-FOS, anti-Brn1, anti-TLE4, anti-CDP, anti-ER81 and anti-GAPDH antibodies were purchased from Santa Cruz. Anti-NeuN antibody was purchased from Chemicon. Confocal images (1 μm) were scanned and subjected to three-dimensional reconstruction. LSMetal0 software (Zeiss) was used to calculate the mean synaptophysin intensity. Brain sections with the strongest intensity were scanned first. All other images included in the analysis were scanned using the same settings. Staining was quantified using LSMetal0 software (Zeiss).

Protein extraction and immunoblotting. The hippocampus and forebrain were collected and lysed in RIPA buffer. The lysates were incubated for 15 min on ice and centrifuged for 15 min at 15,000×g at 4° C. The supernatant was collected as cytosolic protein extract. The lysates were subjected to 10% SDS-PAGE followed by immunoblotting.

Extraction of histone proteins. Hippocampus samples were collected and homogenized in 400 μl TX-buffer (50 mM Tris-HCl, pH8.5, 5 mM sodium butyrate). The pellets were resuspended in 0.2M HCl/TX buffer and incubated on ice for 30 mins. Samples were spun down at 14000 rpm, the histone containing supernatants were subjected to western analysis.

Electrophysiological analysis. 3-6 months old HDAC2OE, HDAC2KO or their littermates were killed by cervical dislocation, and hippocampi were rapidly dissected in iced oxygenated artificial CSF (ACSF). Transverse hippocampal slices, 400 μm thick were placed in a chamber and continuously perfused with oxygenated ACSF. A bipolar stimulating electrodes (0.002-inch-diameter nichrome wire; A-M Systems) placed in the stratum radiatum was used to elicit action potentials in CA3 axons. An ACSF-filled glass microelectrode with a resistance between 0.5 and 3 MΩ was placed in the stratum radiatum region of CA1 and was used to record the field excitatory post-synaptic potentials (fEPSP). Data were acquired using HEKA EPC10 and analyzed by patchmaster (HEKA). Peak fEPSP amplitudes from stimulators were required to be at least 2 mV, and stimulus intensity was set to produce 40% of the maximal response. Baseline responses were recorded for 20 min. fEPSP were evoked at the CA1 synapses by stimulating Schaffer collaterals at a low frequency (2 per min) to establish a stable baseline. Immediately following LTP induction with high-frequency stimulation (HFS, 100 Hz, 1 s), slices from HDAC2OE and control mice showed an increase in fEPSP slope and amplitude, suggesting that short-term potentiation (STP) occurs in all groups. For HDAC2KO and its control WT slices, LTP was induced by applying one train of stimuli at 100 Hz for 1 s. For HDAC2OE and its control WT slices, LTP was induced by applying two trains of stimuli at 100 Hz for 1 s, with an interval of 20 s.

Imaging based EGR-1 expression assay for cultured neurons Embryonic cortici (E17) of EGR1-GFP BAC transgenic mice (Genesat Project) were isolated using standard procedures and triturated with trypsin/DNAse digestion. Cortical neurons were plated at a density of 10,000 cells per well in black/clear bottom plates coated with poly-D-lysine (Costar) in neurobasal medium (1.6% B27, 2% glutamax, 1% pen/strep and 5% heat inactivated fetal calf serum) and in neurobasal medium without serum 24 hrs later. Under these culture conditions, the percentage of glia was estimated to be in the range of 5-25. On day 6, HDAC inhibitors or DMSO control (triplicates or quadruplicates) were added to the cultures for approx. 30 hr. BDNF, KCl or forskolin were added to the cultures on day 7 for 8 hrs.

Cell were fixed in 4% PFA/4% sucrose in PBS. Fixative was washed away with PBS (3 wash cycles) and processed for EGR1-GFPimaging. Cells (3,000-5,000 per well) were imaged and analyzed with 5× objective using the Cellomics ArrayScan Image system. The built-in TargetActivation algorithm was optimized to measure average EGR1-GFP expression per cell (mean Fluorescence intensity per cell per well), using the Hoechst dye to mark cells. The data was normalized to control (medium addition).

After imaging, cells were processed for antibody staining: cells were permeabilized with 0.25% TritonX100 (10-15 min). Triton was washed away by 3 PBS wash cycles, cells were blocked in PBS containing 10% goat or horse serum (1 hr, 37° C.). Cells were exposed to anti-acetyl-Lysine-histone H3 or H4 antibody. Then washed 5 times with PBS followed by secondary antibody conjugated to Alexa594, and Hoechst (1 hr, RT). Secondary antibody was washed 5 times with PBS, and assayed on Cellomics ArrayScan Image system.

Chromatin immunoprecipitation (ChIP) ChIP was performed using mouse forebrains fixed with 4% PFA solution and stored at −80° C. prior to use. Brains were chemically cross-linked by the addition of one-tenth volume of fresh 11% formaldehyde solution for 15 min at room temperature, homogenized, resuspended, lysed in lysis buffers, and sonicated to solubilize and shear crosslinked DNA. Sonication conditions vary depending on cells, culture conditions, crosslinking, and equipment. We used a Misonix Sonicator 3000 and sonicated at power 7 for 10×30 s pulses (90 s pause to between pulses) at 4° C. while samples were immersed in an ice bath. The resulting whole-cell extract was incubated overnight at 4° C. with 100 μl of Dynal Protein G magnetic beads that had been preincubated with 10 μg of the appropriate antibody. Beads were washed five times with RIPA buffer and one time with TE containing 50 mM NaCl. Bound complexes were eluted from the beads by heating at 65° C. with occasional vortexing and crosslinking was reversed by overnight incubation at 65° C. Whole-cell extract DNA (reserved from the sonication step) was also treated for crosslink reversal. Immunoprecipitated DNA and whole-cell extract DNA were then purified by treatment with RNaseA, proteinase K, and multiple phenol:chloroform:isoamyl alcohol extractions. Purified DNA samples were normalized and subjected to PCR analysis. Antibodies used for pull downs were: anti-HDAC1 (#31263), anti-HDAC2(#12169) from Abcam; anti-ACH4 (#06-866), anti-ACH3 (#06-599) from Upstate. After IP, recovered chromatin fragments were subjected to semiquantitative PCR or Real-time PCR for 32-40 cycles using primer pairs specific for 150-250 bp segments corresponding to mouse genes promoter regions (regions upstream of the start codon, near the first exon).

Real-time PCR: Real-time PCR was carried out with SYBR-Green-based reagents (Invitrogen, express SYBR GreenER) using a CFX96 real-time PCR Detection system (BioRad). The relative quantities of immunoprecipitated DNA fragments were calculated using the comparative C_{T} method. Results were compared to a standard curve generated by serial dilutions of input DNA. Data were derived from three independent amplifications. Error bars represent standard deviations.

Primer sequences used for PCR:

BDNF PI:
(SEQ ID NO: 3)
5′-TGATCATCACTCACGACCACG-3′
(SEQ ID NO: 4)
5′-CAGCCTCTCTGAGCCAGTTACG-3′
BDNF PII:
(SEQ ID NO: 5)
5′-TGAGGATAGTGGTGGAGTTG-3′
(SEQ ID NO: 6)
5′-TAACCTTTTCCTCCTCC-3′
BDNF PIV:
(SEQ ID NO: 7)
5′-GCGCGGAATTCTGATTCTGGTAAT-3′
(SEQ ID NO: 8)
5′GAGAGGGCTCCACGCTGCCTTGACG-3′
CREB:
(SEQ ID NO: 9)
5′-CTACACCAGCTTCCCCGGT-3′
(SEQ ID NO: 10)
5′-ACGGAAACAGCCGAGCTC-3
PKM zeta (100 bp upstream of the PKMzeta mRNA
initiation site [15], which contains a
cAMP response element (CRE) consensus sequence):
(SEQ ID NO: 11)
5′-TGTTGAGTCTGGGCCCTC-3′
(SEQ ID NO: 12)
5′-CCTGGCCTCCGGACC-3′
Creb binding protein (CBP):
(SEQ ID NO: 13)
5′-CGGGCAGGGGATGAG-3′
(SEQ ID NO: 14)
5′-GCGAGCCAGCGAGGA-3′
Neurexin I:
(SEQ ID NO: 15)
5′-CAGGGCCTTTGTCCTGAATA-3′
(SEQ ID NO: 16)
5′-GCTTTGAATGGGGTTTTGAG-3′
Neurexin III:
(SEQ ID NO: 17)
5′-ACTGAGAGCTAGCCACCCAGAC-3′
(SEQ ID NO: 18)
5′-TTGCCCATTTGTGAATTTGA-3′
PGK1:
(SEQ ID NO: 19)
5′-ACATTTTGGCAACACCGRGAG-3′
(SEQ ID NO: 20)
5′-GAAGTAGCACGTCTCACTAGTCTCGTG-3′
ATF4:
(SEQ ID NO: 21)
5′-GTGATAACCTGGCAGCTTCG-3′
(SEQ ID NO: 22)
5′-GGGGTAACTGTGGCGTTAGA-3′
CaMKIIA:
(SEQ ID NO: 23)
5′-GACCTGGATGCTGACGAAG-3′
(SEQ ID NO: 24)
5′-AGGTGATGGTAGCCATCCTG-3′
p21 (WAP/CIP1):
(SEQ ID NO: 25)
5′-CCACAGTTGGTCAGGGACAG-3′
(SEQ ID NO: 26)
5′-CCCTCCCCTCTGGGAATCTA-3′
EGR-1:
(SEQ ID NO: 27)
5′-GTGCCCACCACTCTTGGAT-3′
(SEQ ID NO: 28)
5′-CGAATCGGCCTCTATTTCAA-3′
Agrin:
(SEQ ID NO: 29)
5′-TTGTAACCAACAGGGGTTGC-3′
(SEQ ID NO: 30)
5′-AGTTGTGGCTAGGGGAGCAC-3′
EGR-2:
(SEQ ID NO: 31)
5′-GGCTGCAAATCGTTCCTG-3′
(SEQ ID NO: 32)
5′-TCGGAGTATTTATGGGCAGGT-3′
GLUTAMATE RECEPTOR 1 PRECURSOR (GLUR-1/AMPA 1)
(SEQ ID NO: 33)
5′-GGAGGAGAGCAGAGGGAGAG-3′
(SEQ ID NO: 34)
5′-TTCCTGCAATTCCTTGCTTG-3′
GLUR-2
(SEQ ID NO: 35)
5′-GCGGTGCTAAAATCGAATGC-3′
(SEQ ID NO: 36)
5′-ACAGAGAGGGGCAGGCAGT-3′
PSD95:
(SEQ ID NO: 37)
5′-CCCCTACCCCTCCTGAGAAT-3′
(SEQ ID NO: 38)
5′-GAGGGGAAGGAGAAGGTTGG-3′
HOMER1:
(SEQ ID NO: 39)
5′-CTGCCTGAGTGTCGTGGAAG-3′
(SEQ ID NO: 40)
3′-ATGATTTCACTCGCGCTGAC3′
P35:
(SEQ ID NO: 41)
5′-GAGGGAGGGCGCTGAGG-3′
(SEQ ID NO: 42)
5′-GCAGCTAGGGAGCTTCTGTCC-3′
CDK5:
(SEQ ID NO: 43)
5′-CGCAGCCTGTTGGACTTTGT-3′
(SEQ ID NO: 44)
3′-GCGTTGCAGAGGAGGTGGTA-3′
SHANK3:
(SEQ ID NO: 45)
5′-TTTTCCAGGTCCCAGTGGTG-3′
(SEQ ID NO: 46)
5′-CCTGCCCACAGTGTCACTCC-3′
SVP:
(SEQ ID NO: 47)
5′-CTAGCCTCCCGAATGGAATG-3′
(SEQ ID NO: 48)
5′-CAGCAGCAGCATCAGCAATG-3′
SYNAPSIN2
(SEQ ID NO: 49)
5′-GGCTTTCCTTCCCTCCACAC3′
(SEQ ID NO: 50)
5′TGTTAGCGAGGGAGCAGTGG3′
BETA-ACTIN:
(SEQ ID NO: 51)
5′-CCCATCGCCAAAACTCTTCA3′
(SEQ ID NO: 52)
5′GGCCACTCGAGCCATAAAAG3′
GAPDH:
(SEQ ID NO: 53)
5′-CTCCCAGGAAGACCCTGCTT-3′
(SEQ ID NO: 54)
5′-GGAACAGGGAGGAGCAGAGA-3′
ARC:
(SEQ ID NO: 55)
5′-CAGCATAAATAGCCGCTGGT-3′
(SEQ ID NO: 56)
5′-GAGTGTGGCAGGCTCGTC-3′
FOS:
(SEQ ID NO: 57)
5′-GAAAGCCTGGGGCGTAGAGT-3′
(SEQ ID NO: 58)
5′-CCTCAGCTGGCGCCTTTAT-3′
CPG15:
(SEQ ID NO: 59)
5′-GCGAGATTTCGTTGAGATCG-3′
(SEQ ID NO: 60)
5′-GGGATGACACGGATTGATTTT-3′
SNK:
(SEQ ID NO: 61)
5′-TTTCCCACGTCCAAAGTCAG-3′
(SEQ ID NO: 62)
5′-GCAGCGAAGCTTTAAATACGC-3′
NR2A:
(SEQ ID NO: 63)
5′-TCGGCTTGGACTGATACGTG-3′
(SEQ ID NO: 64)
5′-AGGATAGACTGCCCCTGCAC-3′
NR2B:
(SEQ ID NO: 65)
5′-CCTTAGGAAGGGGACGCTTT-3′
(SEQ ID NO: 66)
5′-GGCAATTAAGGGTTGGGTTC-3′
TUBULIN:
(SEQ ID NO: 67)
5′-TAGAACCTTCCTGCGGTCGT-3′
(SEQ ID NO: 66)
5-TTTTCTTCTGGGCTGGTCTC-3′

Statistical analysis: The data were analyzed by unpaired student's t test and one-way ANOVA (ANalyis Of VAriance). One-way ANOVA followed by post-hoc Scheffe's test was employed to compare means from several groups. Error bars present S.E.M.

Results

### Example 1

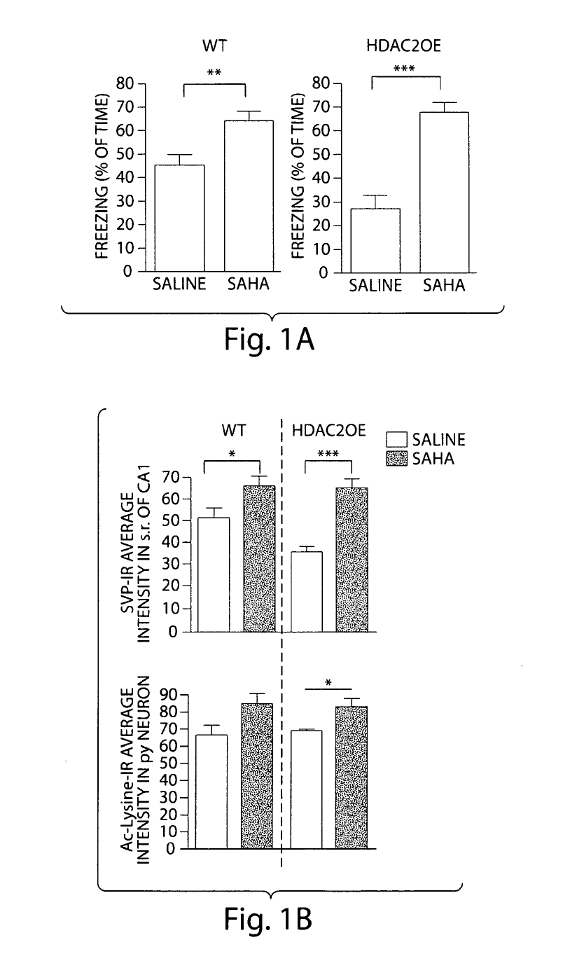

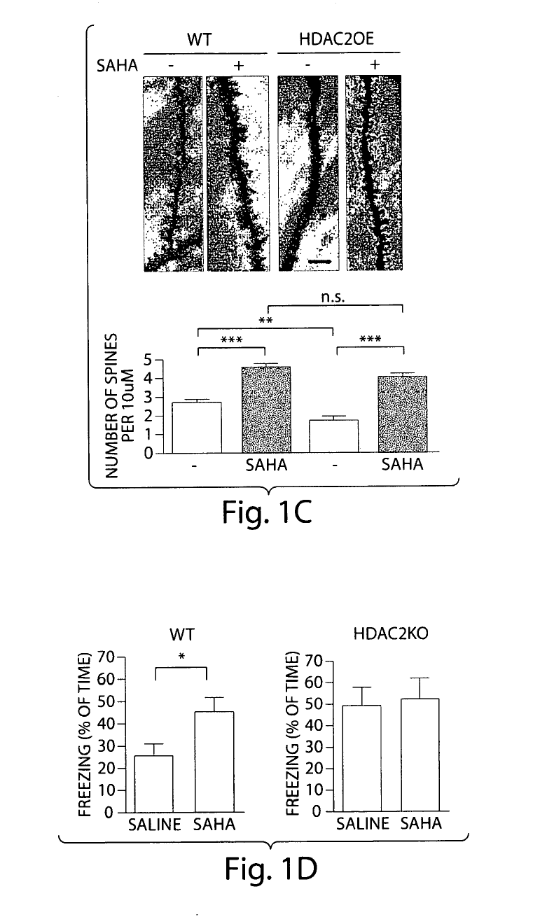

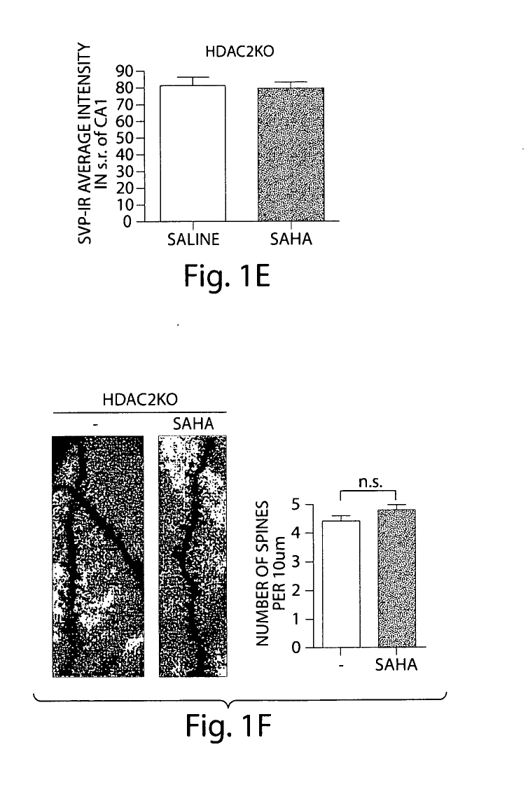

SAHA was administered daily by intraperitoneal (i.p.) injection at 25 mg/kg for 10 days prior to contextual fear conditioning training and memory test. Remarkably, SAHA, but not saline treatment, significantly increased the freezing behavior of HDAC2OE mice (66.7±5.1%, n=12; 26.9±5.9, n=12, p<0.0001, SAHA group versus saline group, FIG. 1A). It should be noted that, in the same training paradigms, SAHA treatment increased the freezing behavior of WT control mice from 44.8±4.7% (n=15, saline control) to 63.9±4.2% (n=12, SAHA treatment). Thus, the freezing levels of HDAC2OE mice after SAHA treatment were comparable to those of the control mice treated with SAHA, despite the fact that saline treated HDAC2OE mice exhibited lower freezing behavior. Concordantly, SARA treatment completely abrogated the decreased dendritic spine and synapses phenotype in HDAC2OE mice (FIG. 1B,C).

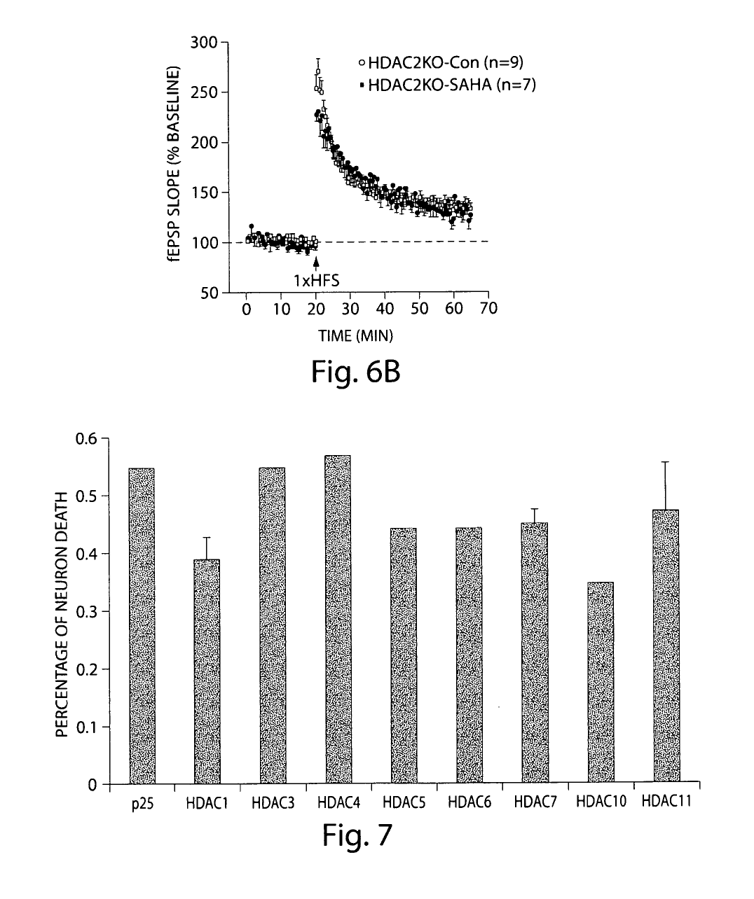

Next, we investigated the effect of SAHA on HDAC2KO mice. As HDAC2KO mice showed markedly increased freezing behavior compared to WT littermates without treatment, we sensitized the assay by lowering the foot shock intensity from 1.0 mA to 0.5 mA to prevent a possible ceiling effect in the memory test. Using this paradigm, we found that SAHA treatment (n=10) induced significantly higher freezing behavior (p=0.0383) compared to saline treatment (n=10) in the WT control mice (45.0±6.9% v.s. 25.0±5.8%, FIG. 1D). However, SAHA treatment did not alter the freezing behavior of the HDAC2KO mice compared to saline treatment (52.1±9.8% v.s. 49.3±8.4%, p=0.8324, n=8 for each group) (FIG. 1D). Furthermore, dendritic spine density of CA1 neurons and synaptophysin staining in the stratum radiatum of the HDAC2KO mice was not significantly affected by SAHA treatment (FIG. 1E,F). Consistently, although SAHA treatment modestly increased LTP in the WT hippocampus, it did not have a detectable effect on LTP in the HDAC2 KO hippocampus (FIG. 6). Thus, HDAC2 KO mice are refractory to synaptogenesis and facilitation of synaptic plasticity and memory formation induced by SAHA. These results strongly suggest that HDAC2 is the major, if not the only target of SAHA in eliciting memory enhancement.

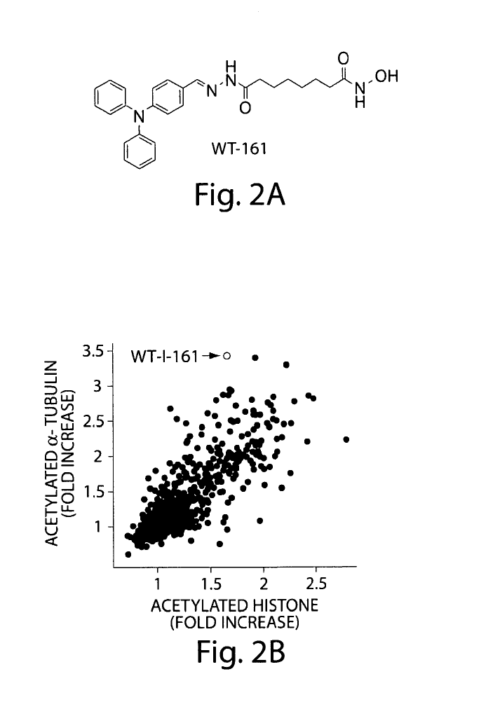

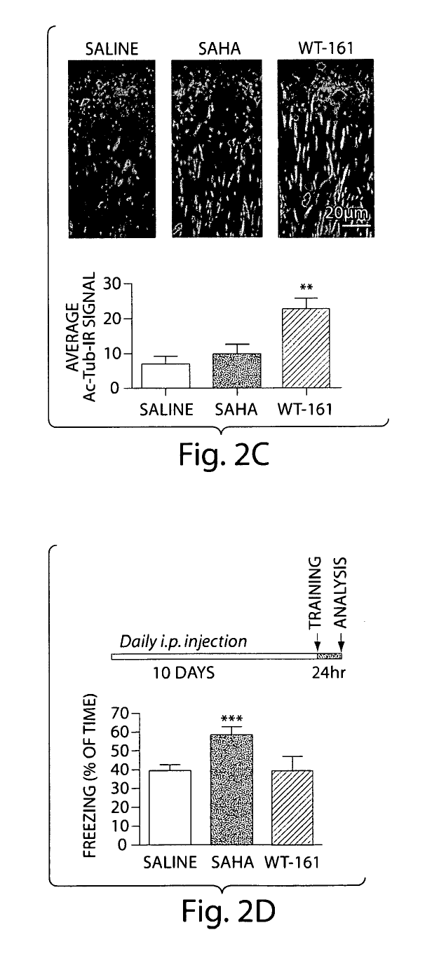

SAHA was initially reported to be a pan-HDACi, although recent studies using recombinant HDACs and in vitro deacetylase assays with appropriate class-specific substrates have revealed that SAHA is a more potent inhibitor of class I HDACs and HDAC6, with very weak to no inhibition of class IIa HDACs, such as HDAC4, 5, and 7. Although SB does not inhibit the activity of HDAC6 in vitro, to directly address the potential importance of this class IIb HDAC, we tested whether selectively inhibiting HDAC6 has any effects on memory formation using the HDACi WT-161 (FIG. 2A,B). α-Tubulin(K40) deacetylation is a known non-histone substrate of HDAC6 that served as specificity control in these experiments. While WT-161 increased α-tubulin(K40) levels in hippocampal pyramidal neurons(FIG. 2C), there was no correlated increase in memory formation (FIG. 2D). This result, and the observed cellular selectivity of SB and WT-161, suggests that HDAC6 inhibition by SAHA might not be involved in HDACi induced memory enhancement. In agreement with these, proteome-wide studies of a SAHA-based affinity probe identified HDAC1 and HDAC2 as the main cellular targets. Thus, class I HDACs, especially HDAC1 and HDAC2, might be the potential target for HDACi induced memory enhancement.

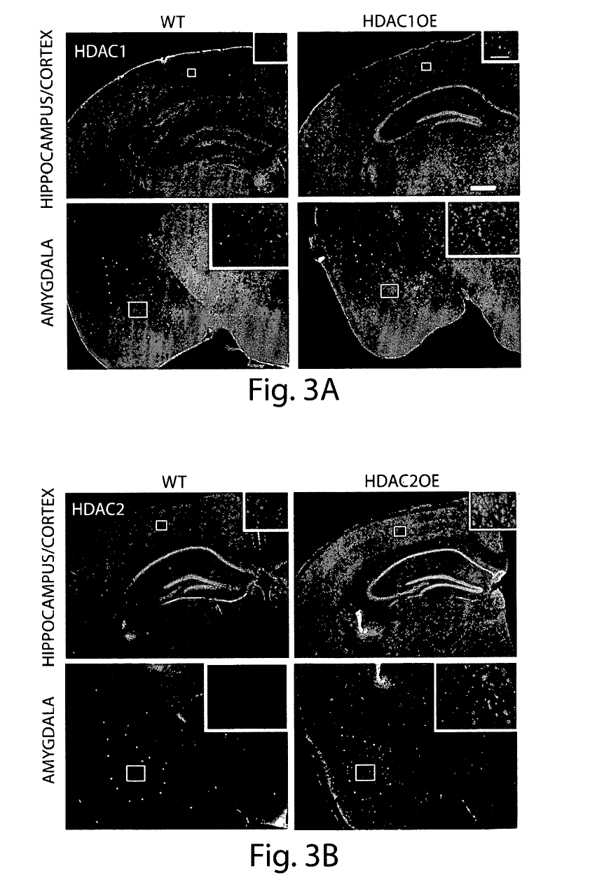

To directly evaluate the physiological role of HDAC1 and HDAC2 in the brain, we generated two mouse lines in which HDAC1 or HDAC2 was over-expressed in neurons. The mouse HDAC1 or HDAC2 coding sequence was placed into exon 1 of the Tau gene, in-frame with the endogenous initiation codon, thereby creating a fusion protein that contains the first 31 amino acids of Tau. Previously, homozygous animals mutant for Tau were shown to be phenotypically indistinguishable from wild-type littermates in memory tests. A 2-3 fold increase in HDAC1 or HDAC2 protein expression in brain of homozygous animals as compared to WT mice was observed in the hippocampus and other areas of the brain (FIG. 3). Consistently, the overall acetylated lysine level was reduced in homozygous HDAC1 (HDAC1OE) and HDAC2 overexpression mice (HDAC2OE), especially in the pyramidal neurons of the hippocampal formation. We found acetylated H4K12, H4K5 but not H3K14 was decreased in brains of HDAC2OE mice (data not shown). In contrast, acetylated α-tubulin(K40) level did not change in the HDAC1OE or HDAC2OE mice. Thus, the HDAC1/2 overexpressing animals exhibited increased histone deacetylation in the brain compared to that of the wildtype (WT) littermates. Importantly, there was no discernable difference in gross brain anatomy or neuronal positioning in the HDAC1/2 overexpressing mice, suggesting that increased HDAC1/2 is not overtly detrimental to brain development or neuronal survival.

Western blots from brain lysate were performed and showed the up-regulation of HDAC1 and HDAC2 respectively in HDAC1 or HDAC2 homozygous over-expression mice (data not shown). Decreased histone acetylation in the hippocampus of HDAC1OE and HDAC2OE mice was observed. Samples from hippocampal histone preparation also showed the reduction of lysine acetylation (at ˜16 KDa) in HDAC1OE mice and HDAC2OE mice.

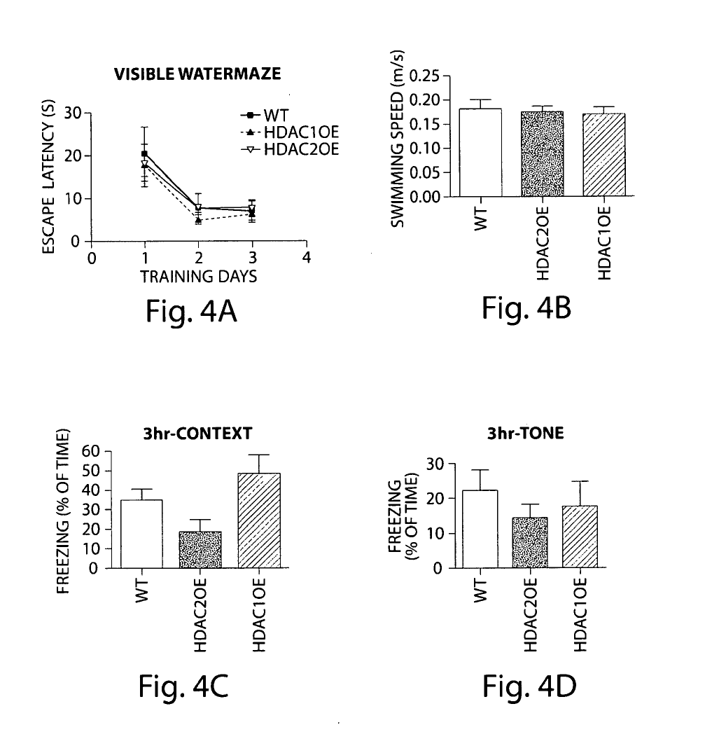

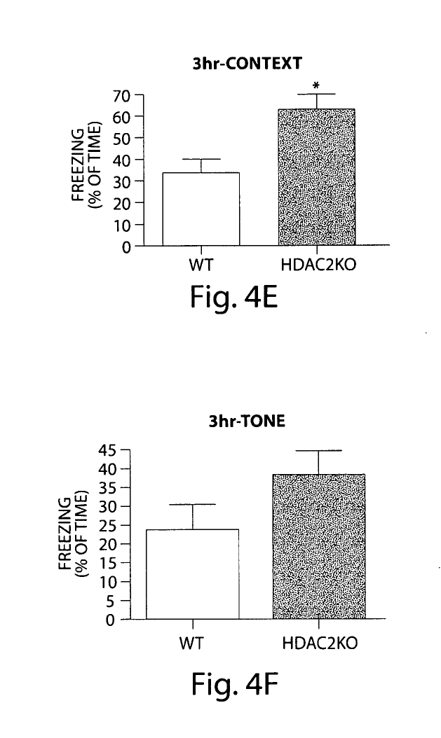

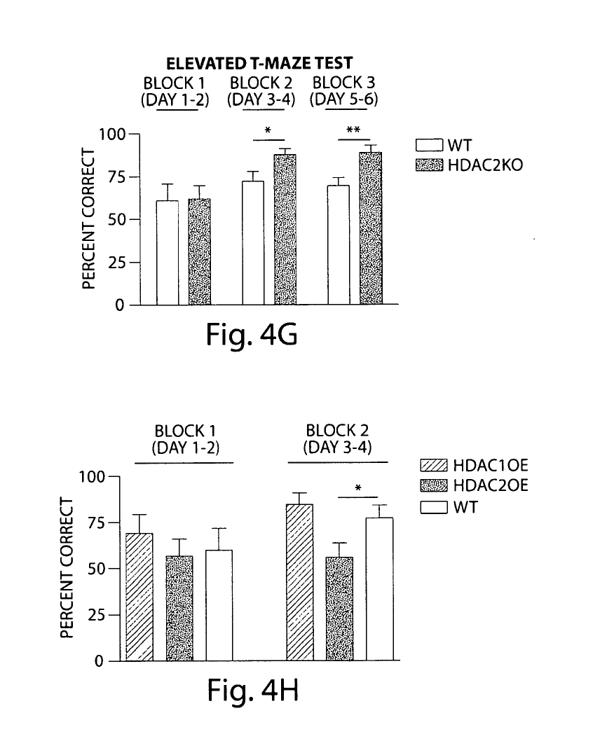

Interestingly, in the short-term memory test, no significant difference could be detected among HDAC1OE, HDAC2OE and WT control in both the context- and tone-dependent fear learning 3 hours after training (FIG. 4B). These observations suggest that HDAC2, but not HDAC1-gain-of-function in the nervous system results in impairment in associative learning. The escape latency and swimming speed were not different among groups in the visible platform test (FIGS. 4A&B), indicating comparable motor and visual function among the various strains. These results revealed a marked reduction of spatial learning of the HDAC2OE mice. Furthermore, HDAC2OE mice but not HDAC1OE mice showed spatial working memory impairment in a T-maze non-matching-to place task (FIG. 4H). Thus, gain of function of HDAC2, but not HDAC1, impairs hippocampus dependent memory formation.

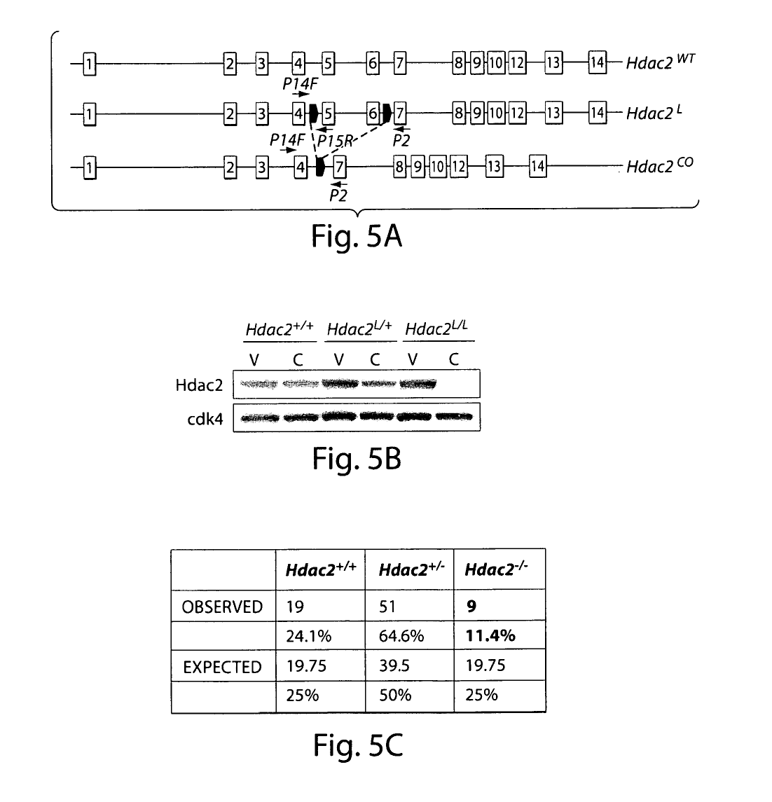

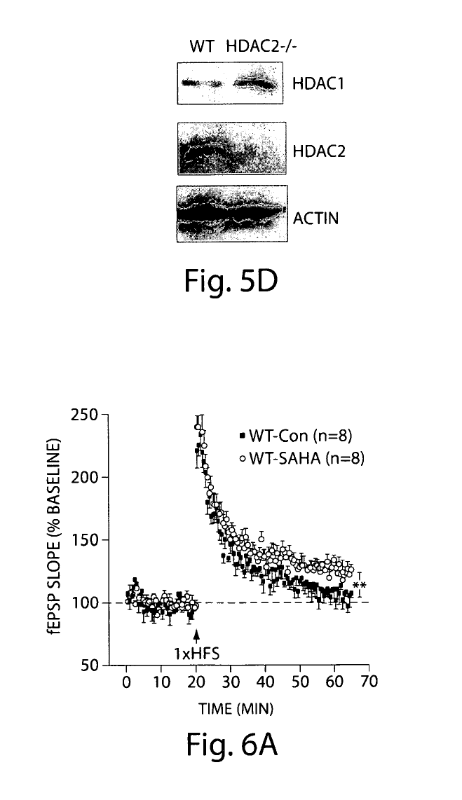

The HDAC 2 gene knockout enhances associative learning. To further investigate the role of HDAC2 in associative learning, HDAC2 deficient mice (HDAC2KO) were generated, by crossing mice carrying a floxed Hdac2 allele with Nestin-Cre transgenic mice. Germ-line deletion of Hdac2 resulted in viable and fertile Hdac2^{+/-} mice with no obvious histological abnormalities up to a year of age (FIG. 5). Crossing Hdac2^{+/-} mice gave rise to viable Hdac2-deficient mice, in which HDAC2 expression was abolished in the brain.

The freezing behavior of HDAC2 knockout (KO) mice and control mice (HDAC2 KO n=10; control, n=10) during the contextual dependent memory test was examined. HDAC2 KO mice showed enhanced fear conditioning.

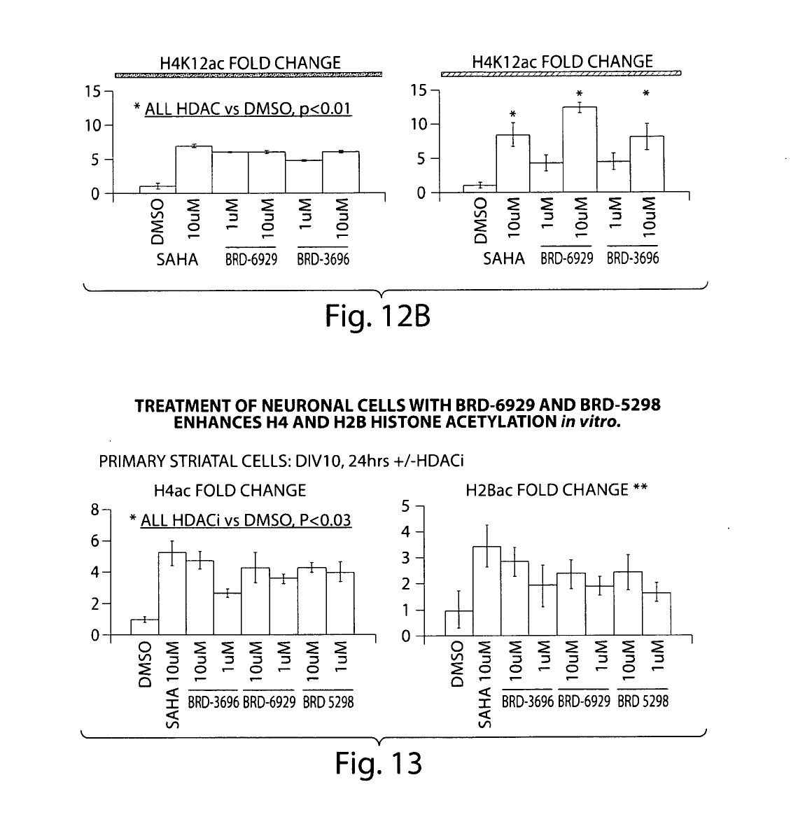

Loss of HDAC2 does not lead to detectable changes in the anatomy or cell positioning in the brain. H4K5, H4K12 and H2B acetylation was significantly increased in the hippocampus of HDAC2KO mice. However, overall acetylation of lysine residues in histone preparation was slightly decreased as revealed by western blot analysis using the acetylated-lysine antibody. This might be the consequence of a compensatory increase of HDAC1 in HDAC2KO mice (FIG. 5D). Remarkably, the HDAC2KO mice (n=9) showed markedly increased freezing behavior as evaluated by the contextual- and tone-dependent fear conditioning paradigm (p=0.0036, p=0.0047, FIG. 12A) 24 hours after training when compared to WT littermates (n=11). In the short-term memory test, HDAC2KO mice (n=9) showed increased freezing behavior (p=0.010 FIG. 4E) comparing to WT littermates (n=8) in contextual dependent conditioning. No difference in the locomotor activity or pain sensation had been detected between these two groups of mice. Thus, HDAC2 loss of function enhanced associative learning. Furthermore, HDAC2KO mice showed a profound spatial working memory improvement in the T-maze non-matching-to place task (p=0.025, two-way ANOVA, FIG. 4G). These data, coupled with the gain of function studies, suggest that HDAC2 may negatively regulate memory formation in mice.

### Example 2

In vitro assays were used to test the protective effects of HDAC overexpression on p25 induced toxicity. Neurons are dissociated from E15.5 cortex and hippocampus. They were transfected with plasmids encoding p25-GFP and Flag-HDACs at DIV4. 24 hrs after transfection, neurons were fixed and processed for IHC. HDAC1,5,6,7 and 10 showed protection (FIG. 7).

In summary, using mouse genetic models, we delineated the functions of HDAC isoforms including class I HDACs such as HDAC1 and HDAC2, and showed evidence that HDAC2 plays a negative role in regulating memory formation. Notably, we identified HDAC2 as the major target of HDACi in facilitating learning and memory. Our observations support the notion that HDAC1 and HDAC2 differentially regulate subset of activity regulated genes or genes implicated in plasticity and memory. This is unexpected, given the fact that HDAC1 and HDAC2 were reported to form functional hetero-dimmers (Grozinger, C. M. & Schreiber, S. L. Chem Biol 9 (1), 3-16 (2002)). It is possible that this is due to the differential distribution of HDAC1 and HDAC2 in the brain as described herein. Alternatively, neuronal HDAC2 and HDAC1 might form distinct complexes with transcriptional co-repressors and therefore are enriched in different regions of the chromatin. Additionally, HDAC2 may differentially target additional non-histone proteins, which may be involved in memory formation. Other possibilities, such as difference in posttranscriptional modification might also contribute to the biochemical/functional dissociation between HDAC1 and HDAC2. It should be noted that HDAC1 deficiency in mice is detrimental, resulting in embryonic lethality. We have also discovered that HDAC1 loss of function in neurons causes DNA damage and cell death. Conversely, HDAC2 deficient mice are viable and exhibit enhanced memory formation. These results not only reveal important distinct functions of HDAC isoforms, and hence, their target genes or non-histone substrates, they also support the discovery that HDAC2 is a suitable target for memory enhancement.
1. Andorfer, C. et al. Cell-cycle reentry and cell death in transgenic mice expressing nonmutant human tau isoforms. J neurosci 25, 5446-5454 (2005).
2. Santacruz, K. et al. Tau suppression in a neurodegenerative mouse model improves memory function. Science 309, 476-481 (2005).
3. Fischer, A., Sananbenesi, F., Pang, P. T., Lu, B. & Tsai, L. H. Opposing roles of transient and prolonged expression of p25 in synaptic plasticity and hippocampus-dependent memory. Neuron 48, 825-838 (2005).

4. Cruz, J. C. & Tsai, L. H. Jekyll and Hyde kinase: roles for Cdk5 in brain development and disease. Curr Opin Neurobiol. 14, 390-394 (2004).
5. Cruz, J. C., Tseng, H. C., Goldman, J. A., Shih, H. & Tsai, L. H. Aberrant Cdk5 activation by p25 triggers pathological events leading to neurodegeneration and neurofibrillary tangles. Neuron 40, 471-83 (2003).
6. Nithianantharajah, J. & Hannan, A. J. Enriched environments, experience-dependent plasticity and disorders of the nervous system. Nat Rev Neurosci 7, 697-709 (2006).
7. Kim, J. J. & Fanselow, M. S. Modality-specific retrograde amnesia of fear. Science 256, 675-7 (1992).
8. Gilmore, E. C. & Herrup, K. Neocortical cell migration: GABAergic neurons and cells in layers I and VI move in a cyclin-dependent kinase 5-independent manner. J Neurosci 21, 9690-700 (2001).
9. Bradshaw, J., Saling, M., Hopwood, M., Anderson, V. & Brodtmann, A. Fluctuating cognition in dementia with Lewy bodies and Alzheimer's disease is qualitatively distinct. J Neurol Neurosurg Psychiatry 75, 382-387 (2004).
10. Palop, J. J., Chin, J. & Mucke, L. A network dysfunction perspective on neurodegenerative diseases. Nature 443, 768-773 (2006).
11. Frankland, P. W., Bontempi, B., Talton, L. E., Kaczmarek, L. & Silva, A. J. The involvement of the anterior cingulate cortex in remote contextual fear memory. Science 304, 881-883 (2004).
12. Need, A. C. & Giese, K. P. Handling and environmental enrichment do not rescue learning and memory impairments in alphaCamKII(T286A) mutant mice. Genes Brain Behay. 2, 132-139 (2003).
13. Tang, Y. P., Wang, H. S., Feng, M., Kyin, Y. Z. & Tsien, J. Z. Differential effects of enrichment on learning and memory function in NR2B transgenic mice. Neuropharmacology, 779-790 (2001).
14. Rampon, C. et al. Effects of environmental enrichment on gene expression in the brain. Proc Natl Acad Sci USA 97, 12880-12884 (2000).
15. Levenson, J. M. et al. Regulation of histone acetylation during memory formation in the hippocampus. J. Biol. Chem. 279, 40545-40559 (2004).
16. Kumar, A. et al. Chromatin remodeling is a key mechanism underlying cocaine-induced plasticity in striatum. Neuron 48, 303-314 (2005).
17. Alarcon, J. M. et al. Chromatin acetylation, memory, and LTP are impaired in CBP+/− mice: a model for the cognitive deficit in Rubinstein-Taybi syndrome and its amelioration. Neuron 42, 947-959 (2004).
18. Korzus, E., Rosenfeld, M. G. & Mayford, M. CBP histone acetyltransferase activity is a critical component of memory consolidation. neuron 42, 961-972 (2004).
19. Voss, H. U. et al. Possible axonal regrowth in late recovery from the minimally conscious state. J Clin Invest. 116, 2005-2011 (2006).
20. van Praag, H., Kempermann, G. & Gage, F. H. Neuronal consequences of environmental enrichment. Nat Rev Neurosci 1, 191-198 (2000).
21. Horn, D., Ruppin, E., Usher, M. & Hermann, M. Neural network modeling of Alzheimer's Disease. Neural Computation 5, 736-749 (1993).
22. Ruppin, E., Reggia, J. A. & Horn, D. Pathogenesis of schizophrenic delusions and hallucinations: a neural model. Schizophr Bull 22, 105-123 (1996).
23. Horn, D., Levy, N. & Ruppin, E. Neuronal-based synaptic compensation: a computational study in Alzheimer's disease. Neuroal comput 8, 1227-1243 (1996).
24. Fischer, A., Sananbenesi, F., Schrick, C., Spiess, J. & Radulovic, J. Cyclin-dependent kinase 5 is required for associative learning. J Neurosci 22, 3700-7. (2002).
25. Frey R R, Wada C K, Garland R B, Curtin M L, Michaelides M R, Li J, Pease L J, Glaser K B, Marcotte P A, Bouska J J, Murphy S S, Davidsen S K. Trifluoromethyl ketones as inhibitors of histone deacetylase. Bioorg Med Chem Lett 2002; 12:3443-3447.
26. Wada C K, Frey R R, Ji Z, Curtin M L, Garland R B, Holms J H, Li J, Pease L J, Guo J, Glaser K B, Marcotte P A, Richardson P L, Murphy S S, Bouska J J, Tapang P, Magoc T J, Albert D H, Davidsen S K, Michaelides M R. Alpha-keto amides as inhibitors of histone deacetylase. Bioorg Med Chem Lett 2003; 13:3331-3335.
27. Haggarty S J, Wong J C, Koeller K M, Butcher R A, Schreiber S L. Multidimensional chemical genetic analysis of diversity-oriented synthesis-derived deacetylase inhibitors using cell-based assays. Chem Biol 2003a; 10:383-396.
28. Guan, Z., et al., Integration of long-term-memory-related synaptic plasticity involves bidirectional regulation of gene expression and chromatin structure. Cell, 2002. 111(4): p. 483-93.
29. Craig, A. M. and Y. Kang, Neurexin-neuroligin signaling in synapse development. Curr Opin Neurobiol, 2007. 17(1): p. 43-52.
30. Chih, B., H. Engelman, and P. Scheiffele, Control of excitatory and inhibitory synapse formation by neuroligins. Science, 2005. 307(5713): p. 1324-8.
31. Silva, A. J., et al., Impaired spatial learning in alpha-calcium-calmodulin kinase II mutant mice. Science, 1992. 257(5067): p. 206-11.
32. Silva, A. J., et al., Deficient hippocampal long-term potentiation in alpha-calcium-calmodulin kinase II mutant mice. Science, 1992. 257(5067): p. 201-6.
33. Cheung, Z. H., A. K. Fu, and N.Y. Ip, Synaptic roles of Cdk5: implications in higher cognitive functions and neurodegenerative diseases. Neuron, 2006. 50(1): p. 13-8.
34. Fischer, A., et al., Cyclin-dependent kinase 5 is required for associative learning. Neurosci, 2002. 22(9): p. 3700-7.
35. Herdegen, T., et al., The KROX-20 transcription factor in the rat central and peripheral nervous systems: novel expression pattern of an immediate early gene-encoded protein. Neuroscience, 1993. 57(1): p. 41-52.
36. van Zundert, B., A. Yoshii, and M. Constantine-Paton, Receptor compartmentalization and trafficking at glutamate synapses: a developmental proposal. Trends Neurosci, 2004. 27(7): p. 428-37.
37. Migaud, M., et al., Enhanced long-term potentiation and impaired learning in mice with mutant postsynaptic density-95 protein. Nature, 1998. 396(6710): p. 433-9.
38. Ksiazek, I., et al., Synapse loss in cortex of agrin-deficient mice after genetic rescue of perinatal death. J Neurosci, 2007. 27(27): p. 7183-95.
39. Vazdarjanova, A., et al., Experience-dependent coincident expression of the effector immediate-early genes arc and Horner 1a in hippocampal and neocortical neuronal networks. J Neurosci, 2002. 22(23): p. 10067-71.
40. Bottai, D., et al., Synaptic activity-induced conversion of intronic to exonic sequence in Horner 1 immediate early gene expression. J Neurosci, 2002. 22(1): p. 167-75.
41. Jaubert, P. J., et al., Complex, multimodal behavioral profile of the Horner1 knockout mouse. Genes Brain Behav, 2007. 6(2): p. 141-54.
42. Vazdarjanova, A., et al., Spatial exploration induces ARC, a plasticity-related immediate-early gene, only in calcium/calmodulin-dependent protein kinase II-positive principal excitatory and inhibitory neurons of the rat forebrain. J Comp Neurol, 2006. 498(3): p. 317-29.
43. Chowdhury, S., et al., Arc/Arg3.1 interacts with the endocytic machinery to regulate AMPA receptor trafficking. Neuron, 2006. 52(3): p. 445-59.
44. Greenberg, M. E., A. L. Hermanowski, and E. B. Ziff, Effect of protein synthesis inhibitors on growth factor activation of c-fos, c-myc, and actin gene transcription. Mol Cell Biol, 1986. 6(4): p. 1050-7.
45. Fleischmann, A., et al., Impaired long-term memory and NR2A-type NMDA receptor-dependent synaptic plasticity in mice lacking c-Fos in the CNS. J Neurosci, 2003. 23(27): p. 9116-22.
46. Matsuo, N., L. Reijmers, and M. Mayford, Spine-type-specific recruitment of newly synthesized AMPA receptors with learning. Science, 2008. 319(5866): p. 1104-7.
47. Tarsa, L. and Y. Goda, Synaptophysin regulates activity-dependent synapse formation in cultured hippocampal neurons. Proc Natl Acad Sci USA, 2002. 99(2): p. 1012-6.
48. Samigullin, D., et al., Regulation of transmitter release by synapsin II in mouse motor terminals. J Physiol, 2004. 561(Pt 1): p. 149-58.
49. Hung, A. Y., et al., Smaller dendritic spines, weaker synaptic transmission, but enhanced spatial learning in mice lacking Shank1. J Neurosci, 2008. 28(7): p. 1697-708.
50. Ehlers, M. D., Molecular morphogens for dendritic spines. Trends Neurosci, 2002. 25(2): p. 64-7.
51. Ohshima, T., et al., Impairment of hippocampal long-term depression and defective spatial learning and memory in p35 mice. J Neurochem, 2005. 94(4): p. 917-25.
52. Cantallops, I., K. Haas, and H. T. Cline, Postsynaptic CPG15 promotes synaptic maturation and presynaptic axon arbor elaboration in vivo. Nat Neurosci, 2000. 3(10): p. 1004-11.
53. Nedivi, E., G. Y. Wu, and H. T. Cline, Promotion of dendritic growth by CPG15, an activity-induced signaling molecule. Science, 1998. 281(5384): p. 1863-6.
54. Liu, L., et al., Role of NMDA receptor subtypes in governing the direction of hippocampal synaptic plasticity. Science, 2004. 304(5673): p. 1021-4.
55. Zhao, M. G., et al., Roles of NMDA NR2B subtype receptor in prefrontal long-term potentiation and contextual fear memory. Neuron, 2005. 47(6): p. 859-72.
56. Sprengel, R., et al., Importance of the intracellular domain of NR2 subunits for NMDA receptor function in vivo. Cell, 1998. 92(2): p. 279-89.
57. Chung, H. J., et al., Requirement of AMPA receptor GluR2 phosphorylation for cerebellar long-term depression. Science, 2003. 300(5626): p. 1751-5.
58. Pak, D. T. and M. Sheng, Targeted protein degradation and synapse remodeling by an inducible protein kinase. Science, 2003. 302(5649): p. 1368-73.

### Example 3

### In Vitro Enzymatic Inhibitions Assay Data

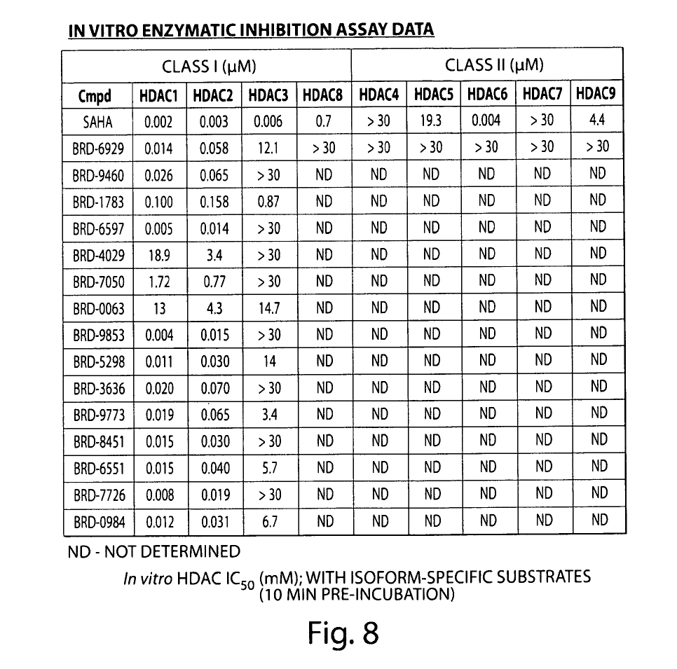

The enzymatic inhibitory activity of multiple HDAC inhibitors was assayed against several of the known HDAC isoforms and is shown in FIG. 8. SAHA was included as a reference mixed class I-class II inhibitor. BRD-6929 demonstrates that this class of compounds does not inhibit HDAC8 or the Class II HDAC enzymes. All of the BRD numbered compounds are derived from the ortho-anilide class of compounds. Not all compounds from this class are expected to bind the class II HDACs.

### Example 4

### In Vitro Cellular Data in Non-Neuronal Cell Lines

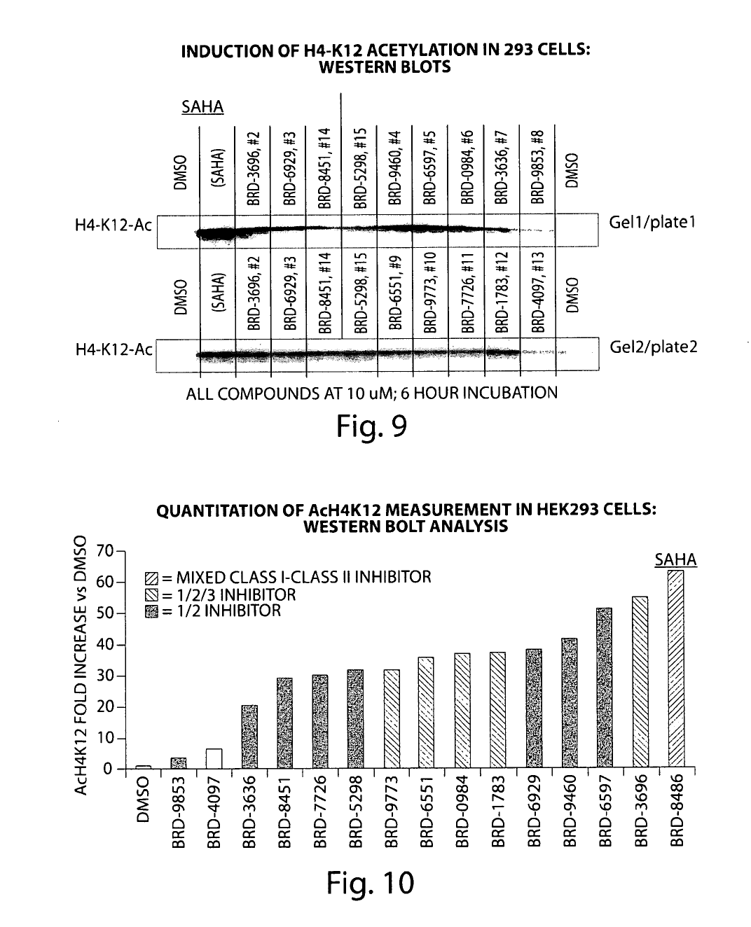

Standard western blotting methods were used to measure the effects of HDAC inhibitors on histone acetylation marks in HeLa cell lysate. Series of compounds were incubated with whole HEK293 cells at 10 uM for a 6 hour time period. Western blot showed increased acetylation levels over DMSO controls using anti-acetyl H4K12 antibodies and horseradish peroxidase conjugated secondary antibody along with a luminol-based substrate (FIG. 9). This demonstrates cellular HDAC activity of these analogs and the increase in acetylation in the specific mark, H4K12. Quantification of the raw western data (FIG. 10) established that relative to the DMSO control, multiple selectivity profiles are effective in increasing H4K12 acetylation levels, and that HDAC 1,2 and HDAC 1,2,3 selective inhibitors have robust HDAC activity in whole cells on a specific histone loci (H4K12).

BRD-9853 showed minimal activity in this cell line. BRD-4097 was the negative control. This is a benzamide with minimal HDAC inhibitory activity.

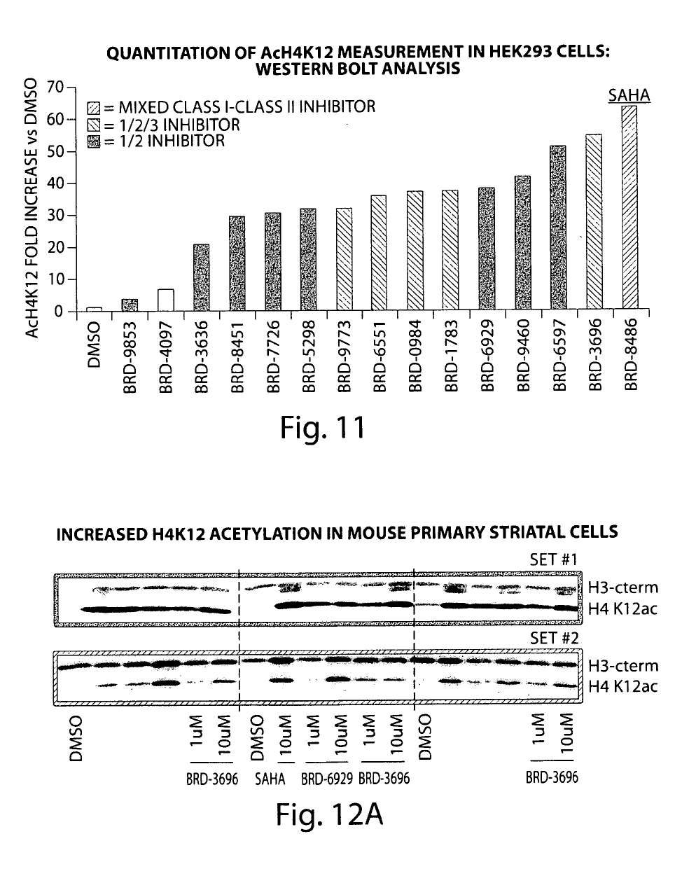

Standard western blotting methods were also used to measure the effects of HDAC inhibitors on histone acetylation marks in HeLa cell lysate. Quantification of western blots in HeLa cells and the effect of compound treatment on the levels of H4K12 acetylation is shown in FIG. 11. Relative to the DMSO control, varying degrees of acetylation were observed. HDAC1,2 and HDAC1,2,3 selective compounds were found to be effective at increasing the acetylation at the H4K12 loci.

### Example 5

### Functional Measures of BRD-6929 Cellular HDAC Activity

FIG. 12 demonstrates western blots of primary striatal cells isolated from mouse brain that have been treated with HDAC inhibitors. Two sets of data with 3 independent samples/set are presented. Histograms representing the quantification of westerns are also shown. Relative to DMSO controls, BRD-6929 has a significant effect on the acetylation levels of histone locus H4K12. BRD-6929 treatment results in a 5-10 fold increase at 1 and 10 uM. BRD-6929 is an HDAC 1,2 selective compound, and has 200× selectivity for HDAC1,2 vs. HDAC3. This demonstrates that an HDAC1,2 selective compound can effectively increase acetylation marks associated with HDAC2 inhibition and memory, H4K12. In this case the data was compared to controls: SAHA and BRD-3696 (CI-994). An HDAC1,2 selective compound is as effective at increasing acetylation as an HDAC1,2,3 inhibitor and a pan inhibitor (i.e. SAHA). Inhibiting HDAC1,2 is sufficient to effect increased acetylation at this histone locus.

FIG. 13 shows histograms representing the quantification of western gel analysis examining additional acetylation marks in primary striatal cells. Four compounds were tested including CI-994 (BRD-3696) and SAHA. Relative to DMSO controls, BRD-6929 and BRD-5298 have significantly increased tetra-acetylated H4. Both compounds also show a trend toward increasing tetra-acetylated H2B. BRD-6929 and BRD-5298 treatment results in a 2-5 fold increase in both marks at 1 and 10 uM. This data demonstrates that HDAC 1,2 specific compounds (BRD-6929, 5298) are effective in increasing a specific acetylation associated with the inhibition of HDAC2 and learning and memory.

### Example 6

### In Vitro Data with Brd-6929 in Neuronal Cell Lines (Immunofluorescent Analysis)

Materials and Methods:

Day 1:

1) Compounds were pin transferred from 384-well plates (Abgene) using a 185 nl pin tool using a no touch bottom protocol.

Day 2: After ˜24 hour compound treatment—

1) Media was aspirated using a plate washer (Tecan) protocol that leaves ˜5 ul residual volume and without touching the bottom of plates); or alternatively, wells were gently aspirated to remove media with 12-channel aspirator wand.

2) A multichannel pipet or use liquid handling system (e.g. Combi, standard tubing; slow speed) was used to add 75 ul formaldehyde (4% in PBS) and wells incubated 10 min at room temperature.

3) Formaldehyde was aspirated and cells rinsed 3 times with 100 ul PBS;

4) PBS was aspirated and 100 ul blocking/permeablization buffer (0.1% Triton-X100, 2% BSA, in PBS) added and wells incubate 1 hour at room temperature.

5) Blocking buffer was aspirated and 50 ul primary antibody diluted 1:500 in blocking buffer was added and wells incubated overnight at 4 degrees.

Day 3:

1) Primary antibody was aspirated and wells rinsed 3 times with 100 ul blocking buffer

2) 50 ul of secondary antibody diluted 1:500 and with Hoeschst (1:1000 from 10 mg/mL (16 mM) stock) added and wells incubated 1.5 hours at room temperature covered in foil to prevent photobleaching.

3) Wells were rinsed 3 times with 100 ul PBS, and a 100 uls of PBS added and the plates, sealed

4) Plates were then read on Acumen/IX Micro

5) Plates were stored at 4 degrees.

Results:

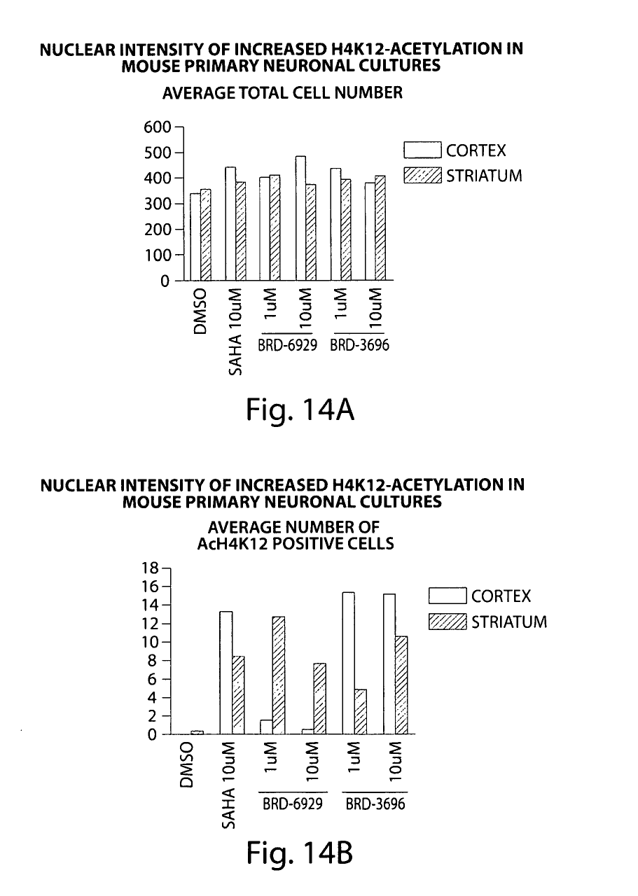

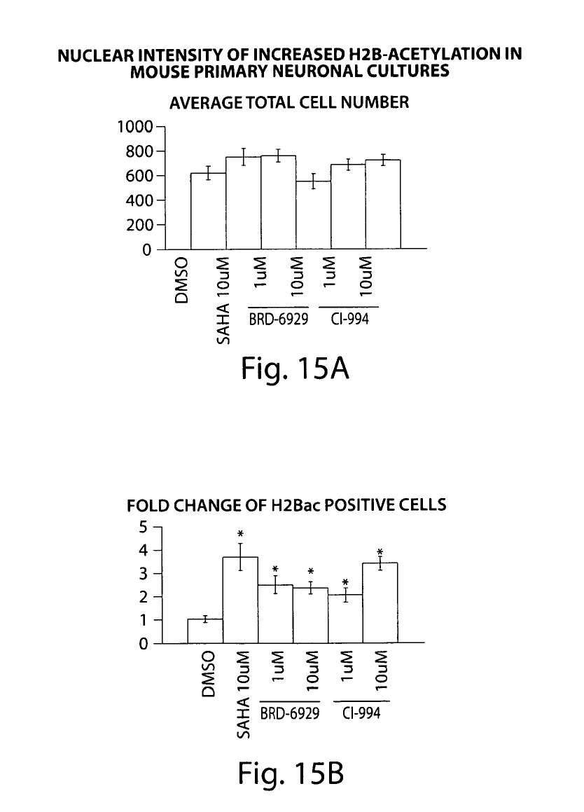

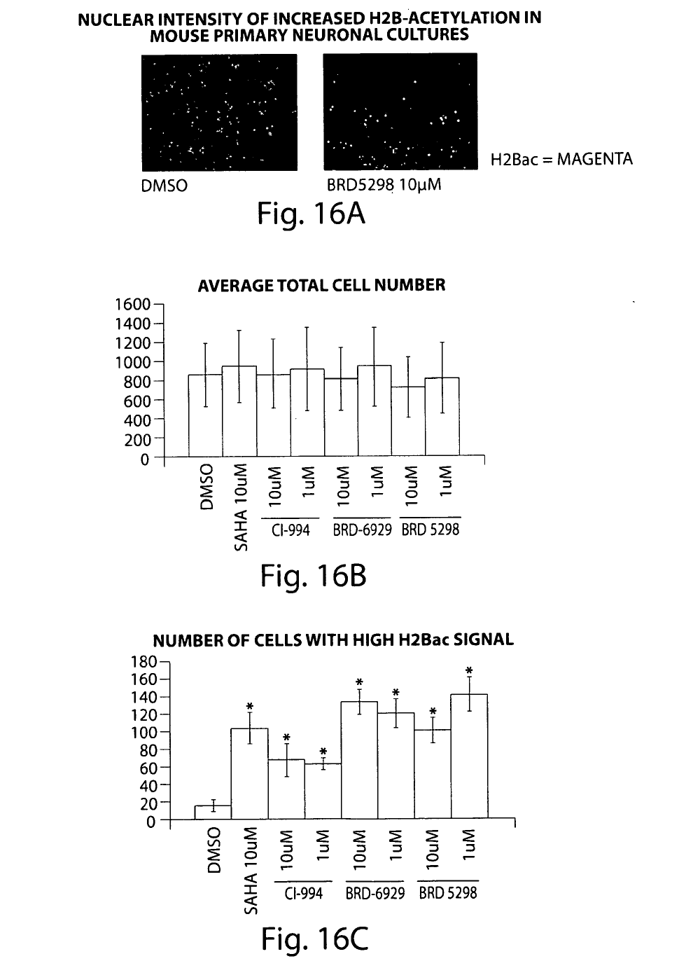

BRD-6929 at 1 and 10 uM does not cause an increase or decrease in overall cell number after 6 h incubation in brain region specific primary cultures (cortex and striatum). BRD-6929 at 10 uM causes an increase in H4K12 acetylation after 6 h incubation in brain region specific primary cultures (striatum) (FIG. 14). BRD-6929 and BRD-5298 (HDAC1,2 selective inhibitors) at 1 and 10 uM cause a significant increase in H2B acetylation after 6 h incubation in primary neuronal cell cultures (FIGS. 15, 16). This demonstrates that HDAC 1,2 selective compounds are effective in increasing the acetylation at the specific histone locus H2B. Increased acetylation of this histone locus is associated with the inhibition or modulation of HDAC2 and learning and memory. To our knowledge there are no reports of compounds with this HDAC inhibitory selectivity eliciting these specific marks in this specific cell type.

### Example 7

### Concentration-Time Curve of BRD-6929 in Plasma and Brain

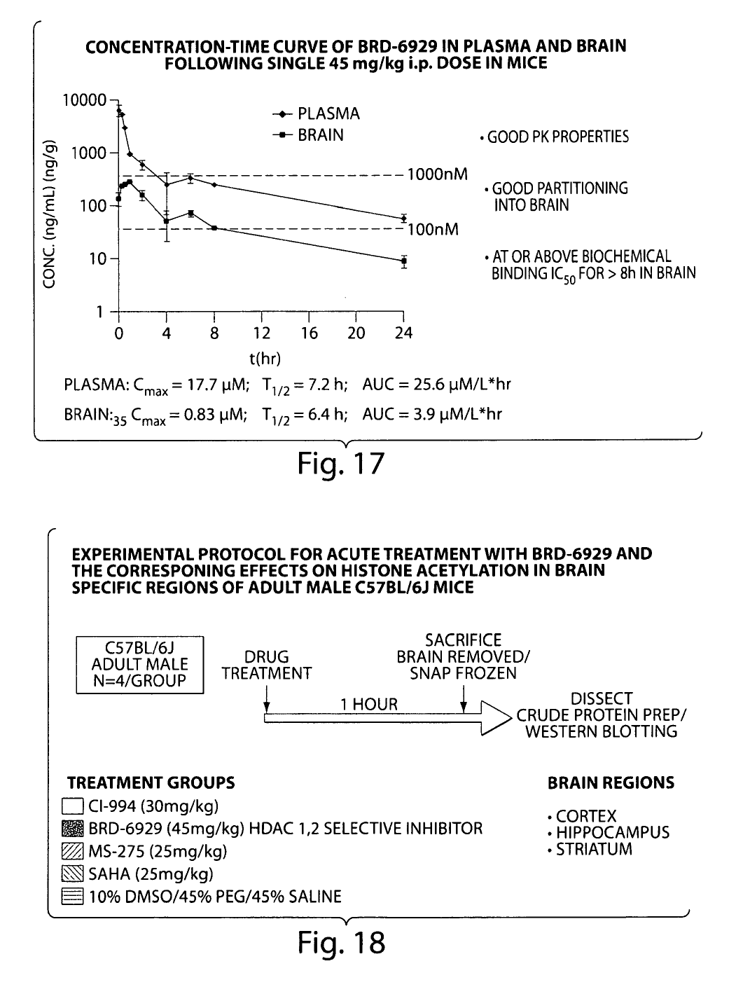

FIG. 17 represents a summary of the pharmacokinetic data after a single dose of 45 mg/kg BRD-6929 administered systemically via intraperitoneal injection. The concentration time curves for BRD-6929 in the plasma and brain of C-57 mice from 5 min to 24 h are shown. This data demonstrates that BRD-6929 crosses the blood-brain barrier and achieves concentrations in excess of its HDAC 1 and 2 IC50 in whole brain. The brain Cₘₐₓ(0.83 uM) and the AUC (3.9 uM) levels are well above effective in vitro concentrations necessary for enzymatic inhibition.

### Example 8

### Increase in Acetylation Marks in Brain Specific Regions Related to Learning and Memory after Acute Dosing in Mice

The experimental protocol for acute treatment with BRD-6929 and the corresponding effects on histone acetylation in brain specific regions of adult male C57BL/6J mice is shown in FIG. 18. Crude Protein Lysis Protocol for western blot analysis of specific brain sections.

1. For dissected, frozen brain tissue:

a. On ice, thaw frozen tissue and immediately homogenize carefully in 250 uL of ice-cold Suspension Buffer.

(100 uL was used for tissue approx. 2-3 mm3; adjust as needed)

1.5 mL disposable pestles (Fisher cat #03-392-100)

b. As soon as possible, add an equal volume of 2×SDS gel-loading buffer, pipetting up and down to mix.

2. Place the sample at 95° C. for 5 min.

3. Shear viscous chromosomal DNA by smoothly passaging through 23-25 gauge hypodermic needle (2-3×) or by sonicating briefly (Al used the needle method and it worked fine). Avoid foaming/bubbles.

4. Centrifuge the sample at 10,000 g for 10 min at room temperature, transferring supernatant to fresh tube.

5. Aliquot sample as needed based on protein concentration.

Suspension Buffer:

0.1M NaCl, 0.01M TrisCl (pH 7.6), 0.001M EDTA (pH 8.0) (buffer to this point can be prepared ahead, room temp. storage) Just before use, add: 1× phosphatase/protease inhibitor cocktail (ex. ThermoFisher “HALT,” cat #78440) 5 mM Sodium Butyrate (HDAC inhibitor).

2×SDS Gel-Loading Buffer:

100 mM TrisC1 (pH 6.8), 4% SDS, 20% glycerol (buffer to this point can be prepared ahead, room temp. storage) Just before use, add: 200 mM dithiothreitol (from 1M stock) 5 mM Sodium Butyrate (1-1DAC inhibitor).

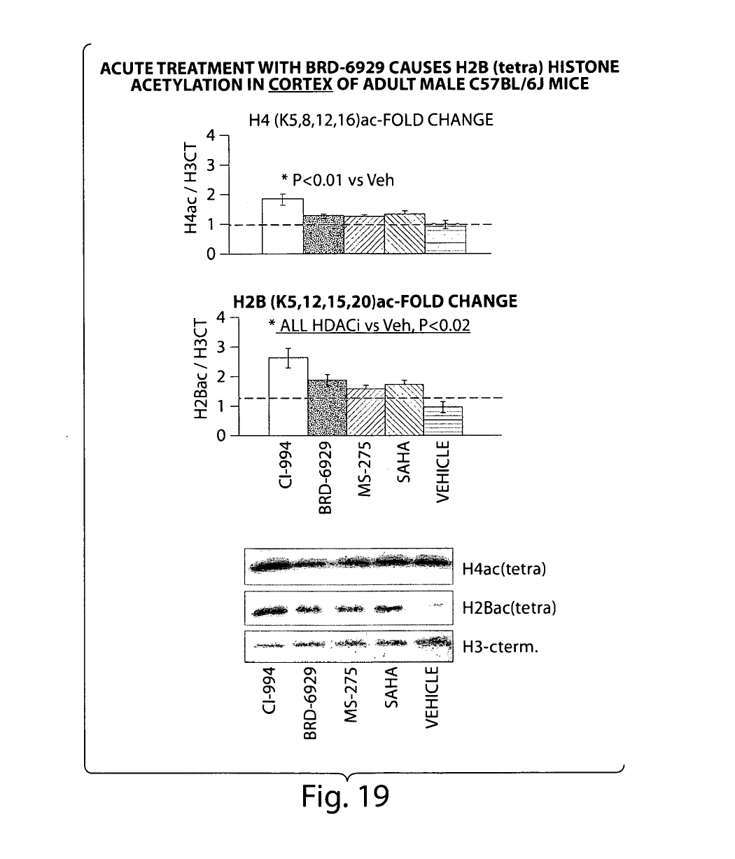

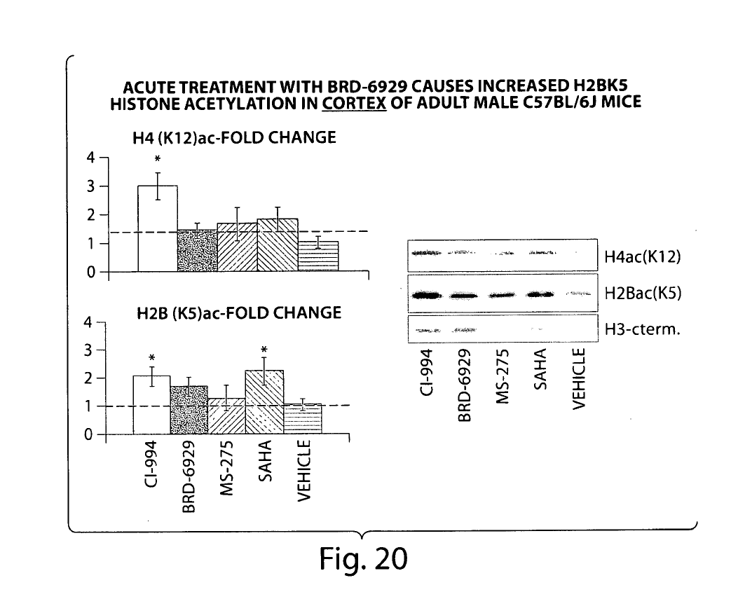

Results: BRD-6929 causes a significant increase in the levels of tetra-acetylated H2B in the cortex of mice (FIG. 19). This demonstrates that BRD-6929 is a functional inhibitor of HDACs in the cortex after a single dose given systemically. BRD-6929 causes a 1.5-2 fold increase in the acetylation levels for H2BK5 (FIG. 20). This acetylation mark has been associated with increased learning and memory. These experiments demonstrate that BRD-6929, an HDAC 1,2 selective inhibitor, has entered the brain, and the nucleus of cells located in specific brain regions associated with learning and memory. Moreover, BRD-6929 causes an increase in specific acetylation marks which have also been associated with learning and memory effects. To our knowledge, it has not been demonstrated that a compound with this high level of HDAC 1,2 selective inhibition is efficacious in increasing acetylation levels in the brain.

### Example 9

### Increase in Acetylation Marks in Whole Brain after Chronic Administration of BRD-6929

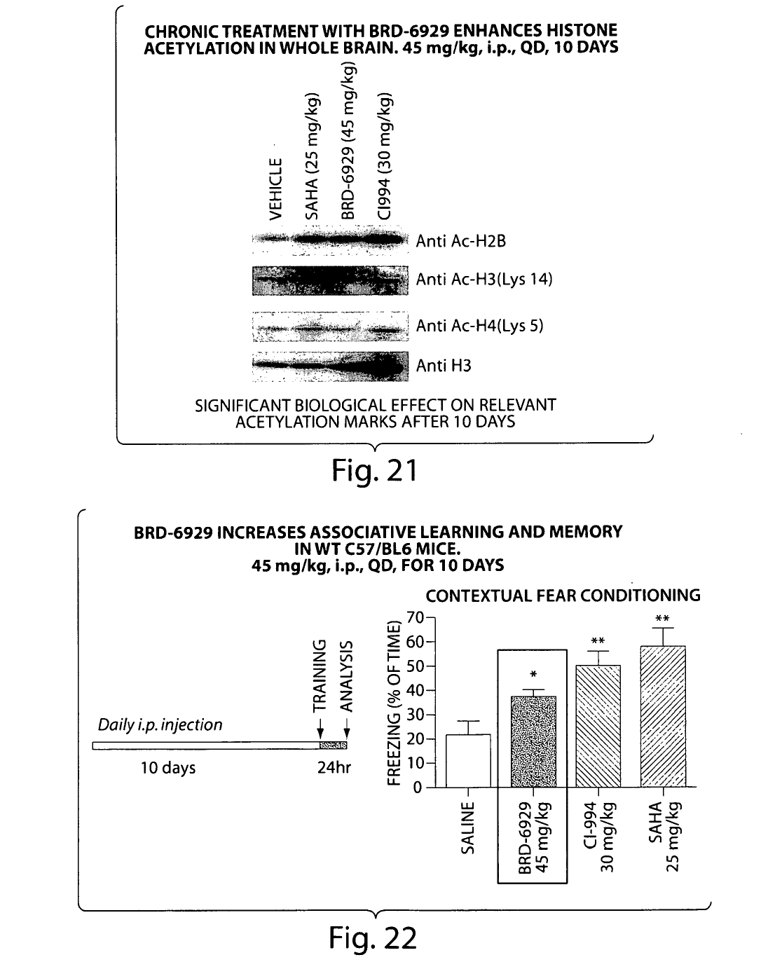

Western gel analysis demonstrated that even after chronic administration of BRD-6929, every day for 10 days, BRD-6929 can still exert an effect on acetylation levels in the brains of mice. The western blot showed an increase in tetra-acetylated H2B relative to the vehicle control (FIG. 21). This demonstrates that an HDAC 1,2 selective compound can effectively increase acetylation levels of specific acetylation marks (tetra-acetylated H2B) in the brain after chronic injection.

### Example 10

### Behavioral Data in Mice: Phenotypes that Correspond to Improved Memory and Cognition

C57/BL6 WT mice were injected with vehicle or BRD-6929 for 10 days. On day 11, mice were trained in contextual fear conditioning paradigm (Training consisted of a 3 min exposure of mice to the conditioning box (context, TSE) followed by a foot shock (2 sec, 0.8 mA, constant current). One hour after training, mice were injected with BRD-6929 or vehicle. On day 12, mice were returned to the training box and the freezing behavior were monitored and recorded.

Result: A 45 mg/kg dose of BRD-6929 given every day for 10 days improved the memory of mice in a contextual fear conditioning paradigm as measured by % time freezing (FIG. 22). To our knowledge, this effect has not been reported previously for an HDAC1,2 selective compound or for this class of compounds under any conditions. It was quite unexpected that this HDAC 1,2, selective inhibitor would be efficacious.

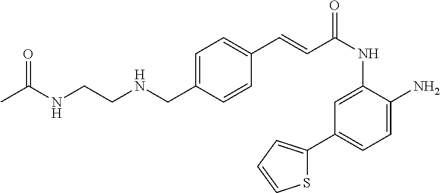

### Example 11

### Synthesis of (E)-3-(4-((2-acetamidoethylamino)methyl)phenyl)-N-(2-amino-5-(thiophen-2-yl)phenyl)acrylamide (BRD-9460)

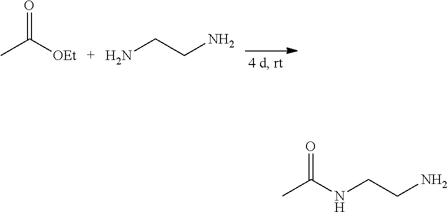

A mixture of ethyl acetate (3.0 g, 34.1 mmol, 1.0 eq) and ethylenediamine (6.14 g, 102 mmol, 3.0 eq) was stirred at room temperature for 4 days. The reaction mixture was then concentrated in vacuo. The product was purified by flash chromatography (silica gel, 1% ammonia/49% CH₂Cl₂/50% MeOH) to afford the desired product as a yellow oil (2.0 g, 57% yield).

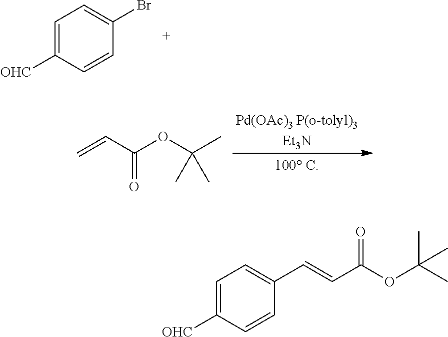

A mixture of 4-bromobenzaldehyde (9.25 g, 50.0 mmol, 1.0 eq), tert-butyl acrylate (8.01 g, 62.5 mmom, 1.25 eq), triethylamine (10.12 g, 100 mmol, 2.0 eq), triacetoxylpalladium (0.14 g, 0.5 mmol, 0.01 eq) and tri-o-tolylphosphine (0.609 g, 2.0 mmol, 0.04 eq) was heated at 100° C. for 2 h under nitrogen atmosphere. The reaction mixture was then diluted with water and extracted with ethyl acetate. The aqueous layer was adjusted to pH˜3 with a 1M aqueous solution of HCl. The product was extracted with ethyl acetate. The combined organic layer were filtered, dried over sodium sulfate and concentrated in vacuo to give the desired product as a yellow solid (10.3 g, 89% yield).

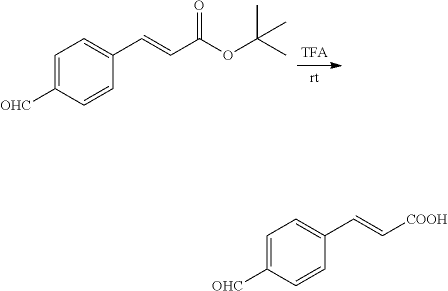

A mixture of tert-butyl cinnamate (10.3 g, 44.3 mmol) in trifluoroacetic (100 mL) acid was stirred at room temperature overnight. The solvent were then removed by evaporation under reduced pressure. The obtained yellow residue was dissolved in a saturated aqueous solution of sodium carbonate. The suspension was filtered and the filtrate was treated with a 3 M aqueous solution of HCl to give a white precipitate. The precipitate was then filtered off and dried to obtain the desired product as a white solid (5.3 g, 67% yield).

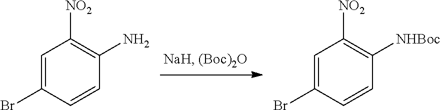

To a stirred solution of 4-bromo-2-nitroaniline (50 g, 230.4 mmol, 1 eq) in DMF (800 mL) was added 60% NaH (6.1 g, 253.4 mmol, 1.1 eq) and (Boc)₂O (60.3 g, 276 mmol, 1.2 eq) in DMF (200 ml) at 0° C. The reaction mixture was stirred room temperature for 5 h. The reaction was then poured into ice-cold water and stirred for 1 h. The obtained solid was filtered and dried under reduced pressure. The crude material was purified by column chromatography (silica gel, 1% EtOAc/hexanes) to give the desired product (41.0 g, 56% yield).

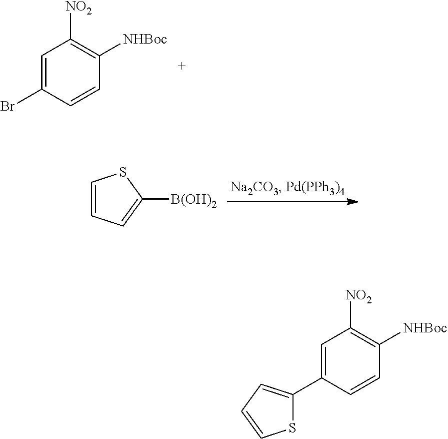

To a stirred solution of compound tert-butyl 4-bromo-2-nitrophenylcarbamate (1 g, 3.15 mmol, 1 eq) in DME (7 mL) was added thiophen-2-ylboronic acid (0.48 g, 3.78 mmol, 1.2 eq), Na₂CO₃ (1.0 g, 9.46 mmol, 3.0 eq), tetrakis(triphenylphosphine)palladium(0) (0.36 g, 0.31 mmol, 0.1 eq) and water (3 mL). The reaction mixture was heated at 90° C. for 18 h. The reaction was diluted with EtOAc and water. The organic layer was separated, dried over sodium sulfate, filtered and concentrated. The crude material was purified by column chromatography (silica gel, 10% EtOAc/hexanes) to afford the desired product (0.51 g, 50% yield).

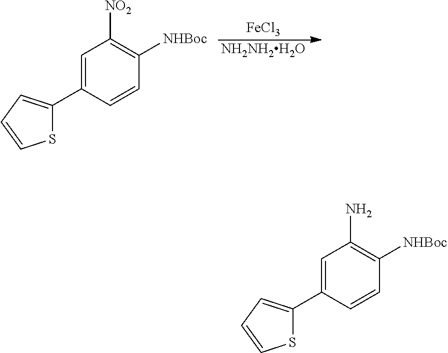

To a solution of tert-butyl 2-nitro-4-(thiophen-2-yl)phenylcarbamate (12.0 g, 37.5 mmol, 1 eq) in methanol (200 mL) was added hydrazine monohydrate (80 mL) and iron (III) chloride (0.37 g, 2.24 mmol, 0.06 eq). The reaction was stirred 80° C. for 1 h. The reaction was then filtered hot over celite and concentrated under reduced pressure. The obtained residue was diluted with water (500 mL) and stirred well. The obtained solid was filtered washed with water then hexanes and dried (10.5 g, 97% yield).

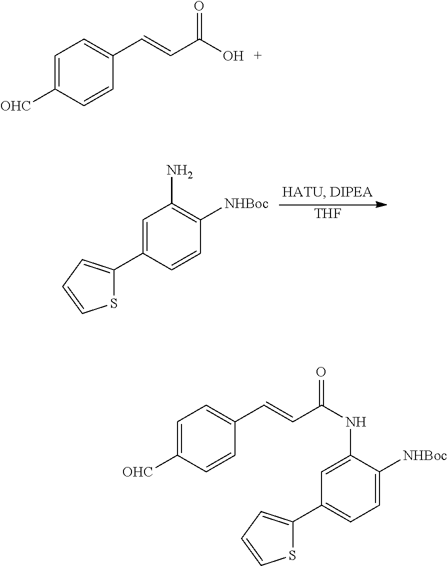

A mixture of (E)-3-(4-formylphenyl)acrylic acid (1.0 g, 5.68 mmol, 1.0 eq), tert-butyl 2-amino-4-(thiophen-2-yl)phenylcarbamate (1.48 g, 5.11 mmol, 0.9 eq) in THF was treated with HATU (2.2 g, 9.13 mmol, 1.0 eq) and DIPEA (1.8 g, 25.3 mmol, 4.46 eq). The resulting mixture was stirred at room temperature for 20 h. The solvents were removed by evaporation. The residue was diluted with ethyl acetate and washed with water, then brine. The organic layer was separated, dried over Na₂SO₄ and concentrated under reduced pressure. The product was purified by column chromatography (silica gel, 50% Petroleum ether/50% CH₂Cl₂) to afford a crude product as a yellow solid (2.2 g, 86% yield).

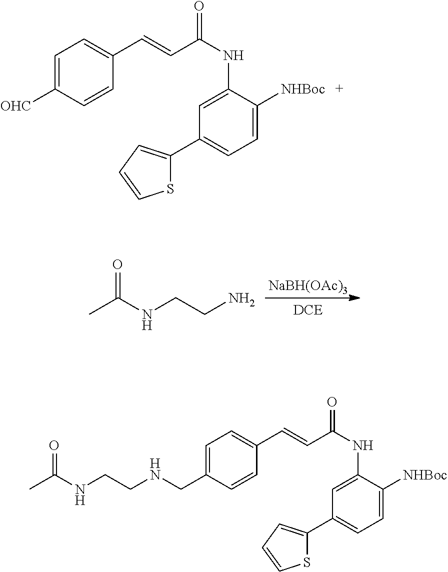

To a mixture of (E)-tert-butyl 2-(3-(4-formylphenyl)acrylamido)-4-(thiophen-2-yl)phenylcarbamate (0.15 g, 0.33 mmol, 1.0 eq) and N-(2-aminoethyl)acetamide (0.07 g, 0.67 mmol, 2.0 eq) in dichloroethane was added NaBH(OAc)₃ (0.43 g, 2.01 mmol, 6.0 eq). The reaction was stirred at room temperature for 20 h. The reaction was concentrated, and ethyl acetate (30 mL) was added. The organic solution was washed with a saturated aqueous solution of sodium bicarbonate (10 mL), then brine (10 mL), the organic layer was concentrated in vacuo, the residue was purified by prep TLC (5% MeOH/CH₂Cl₂) to afford the desired product as a white solid (0.04 g, 22% yield).

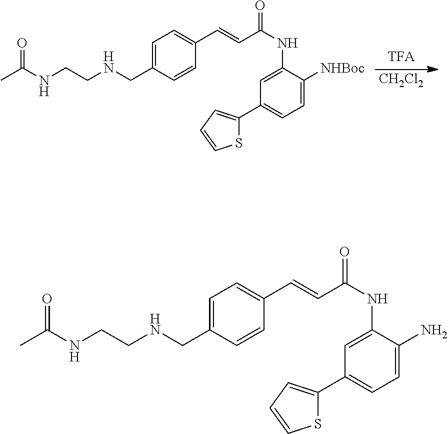

A mixture of (E)-tert-butyl 2-(3-(4-((2-acetamidoethylamino)methyl)phenyl)acrylamido)-4-(thiophen-2-yl)phenylcarbamate (0.04 g, 0.08 mmol) in dichloromethane (4 mL) was treated with trifluoroacetic acid (1 mL) and stirred at room temperature for 1 h. The reaction mixture was quenched with a saturated aqueous solution of bicarbonate and extracted with ethyl acetate. The organic layer was separated, dried over sodium sulfate, filtered and concentrated. The obtained yellow solid was washed with DCM/Hexane and dried under reduced pressure (0.02 g, 55.4% yield). ESI+ MS: m/z (rel intensity) 435 (100, M+H), ¹H NMR (500 MHz, d⁶-DMSO): δ 9.45 (s, 1H), 7.87-7.78 (m, 1H), 7.70 (s, 1H), 7.63-7.52 (m, 3H), 7.45-7.32 (m, 3H), 7.28-7.20 (m, 2H), 7.08-7.02 (m, 1H), 6.89 (d, J=15.5 Hz, 1H), 6.79 (d, J=8 Hz, 1H), 5.22 (s, 2H), 3.74 (s, 2H), 3.14 (d, J=6 Hz, 2H), 2.60-2.42 (m, 2H), 1.79 (s, 3H).

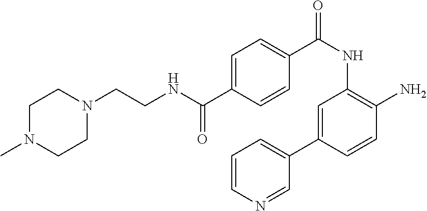

### Synthesis of N1-(2-amino-5-(pyridin-3-yl)phenyl)-N4-(2-(4-methylpiperazin-1-yl)ethyl)terephthalamide (BRD-6551)

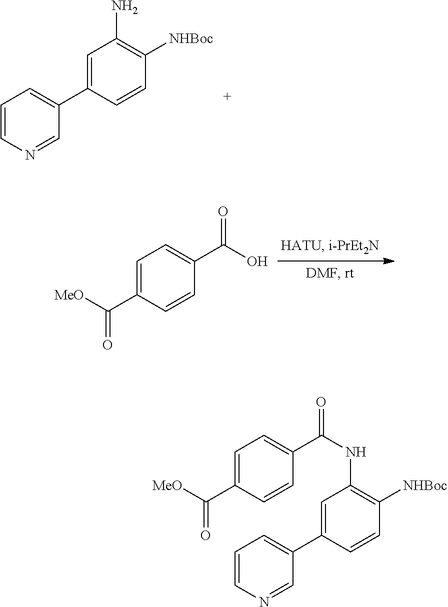

A mixture of tert-butyl 2-amino-4-(pyridin-3-yl)phenylcarbamate (0.50 g, 1.74 mmol, 1 eq) [which was prepared in a similar manner to tert-butyl 2-amino-4-(thiophen-2-yl)phenylcarbamate], 4-(methoxycarbonyl)benzoic acid (0.47 g, 2.62 mmol, 1.5 eq), and BOP (1.4 g, 3.16 mmol, 1.8 eq) in pyridine (5 mL) was stirred at room temperature for 20 h. The solvent was removed by evaporation. The residue was then diluted with a saturated aqueous solution of sodium bicarbonate. The obtained solid was filtered. The crude product purified by column chromatography (silica gel, 2% MeOH/CH₂Cl₂) to give the desired product (0.68 g, 87% yield).

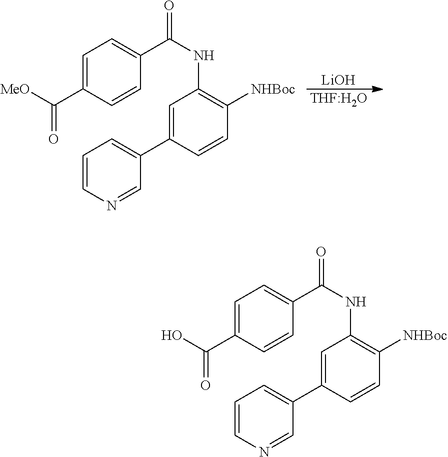

A solution of methyl 4-(2-(tert-butoxycarbonylamino)-5-(pyridin-3-yl)phenylcarbamoyl)benzoate (0.68 g, 1.52 mmol, 1 eq) in THF (10 mL) was treated with a solution of lithium hydroxide (0.18 g, 7.60 mmol, 5.0 eq) in water (10 mL). The reaction was stirred at room temperature 2 h. The reaction was concentrated then diluted with water and adjusted to pH˜3 with citric acid. The obtained solid was filtered and used directly in the next reaction (0.61 g, 93% crude yield).

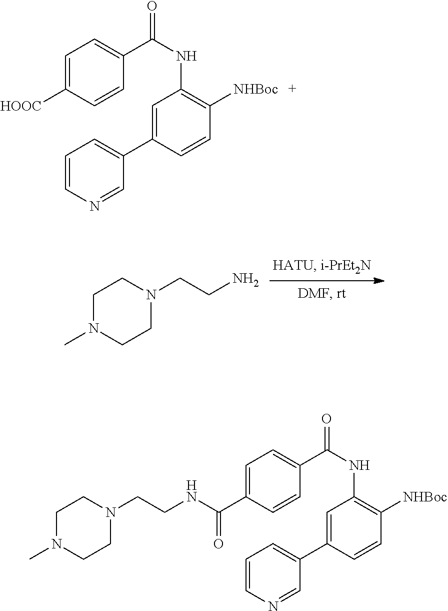

A mixture of 4-(2-(tert-butoxycarbonylamino)-5-(pyridin-3-yl)phenylcarbamoyl)benzoic acid (0.20 g, 0.46 mmol, 1.0 eq), 2-(4-methylpiperazin-1-yl)ethanamine (0.13 g, 0.92 mmol, 2.0 eq) in DMF (4 mL) was treated with HATU (0.35 g, 0.92 mmol, 2.0 eq) and DIPEA (0.20 mL, 1.15 mmol, 2.5 eq). The reaction was stirred at room temperature for 20 h. Water was added. The obtained solid was filtered and dried. The crude product was purified by column chromatography (silica gel, 30% EtOAc/hexanes) to afford the desired product (0.19 g, 77% yield).

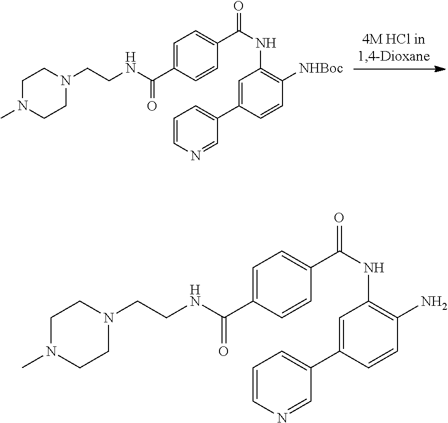

A 4M solution of HCl in 1,4-dioxane (2 mL) was added to a stirred solution of tert-butyl 2-(4-(2-(4-methylpiperazin-1-yl)ethylcarbamoyl)benzamido)-4-(pyridin-3-yl)phenylcarbamate (0.10 g, 0.18 mmol, 1 eq) in methanol (2 mL) at 0° C. The reaction was then warmed to room temperature and stirred for 2 h. The solvents were removed by evaporation and a saturated aqueous solution of sodium bicarbonate was added. The obtained solid was filtered and dried under vacuum to get the desired compound (0.05 g, 60% yield). ESI+ MS: m/z (rel intensity) 459 (96.6, M+H), ¹H NMR (500 MHz, d6-DMSO): δ 9.84 (s, 1H), 8.79 (d, J=1.5 Hz, 1H), 8.53 (t, J=5.5 Hz, 1H), 8.44 (dd, J=4.0, 1.5 Hz, 1H), 8.08 (d, J=8.5 Hz, 2H), 7.95 (d, J=8.5 Hz, 3H), 7.58 (s, 1H), 7.42-7.38 (m, 2H), 6.90 (d, J=8.5 Hz, 1H), 5.23 (bs, 1H), 3.42-3.36 (m, 2H), 2.55-2.20 (m, 10H), 2.15 (s, 3H).

One skilled in the art will recognize that other compounds described below can be prepared in a similar manner to the procedures described above.

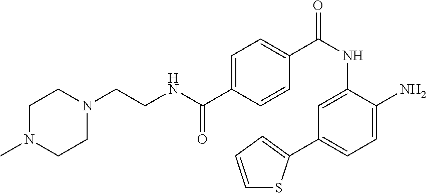

N1-(2-amino-5-(thiophen-2-yl)phenyl)-N4-(2-(4-methylpiperazin-1-yl)ethyl)terephthalamide (BRD-5298) can be prepared by substituting pyridin-3-ylboronic acid with thiophen-2-ylboronic acid. ESI+ MS: m/z (rel intensity) 464 (98.27, M+H).

The following four compounds can be prepared by substituting 2-(4-methylpiperazin-1-yl)ethanamine with 2-phenylethanamine and utilizing the appropriate benzoic acid.

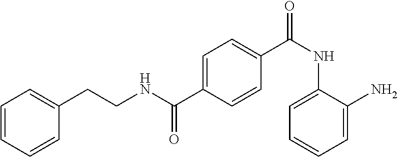

N1-(2-aminophenyl)-N4-phenethylterephthalamide (BRD-1783), ESI+ MS: m/z (rel intensity) 359 (100, M).

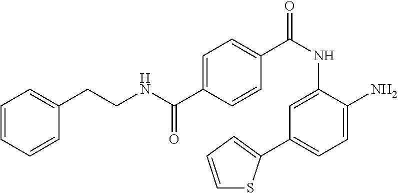

N1-(2-amino-5-(thiophen-2-yl)phenyl)-N4-phenethylterephthalamide (BRD-8451), ESI+ MS: m/z (rel intensity) 442 (99.34, M+H).

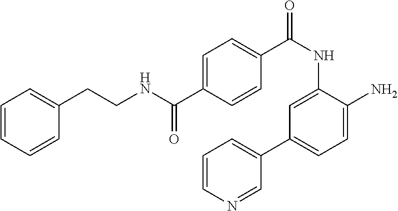

N1-(2-amino-5-(pyridin-3-yl)phenyl)-N4-phenethylterephthalamide (BRD-0984), ESI+ MS: m/z (rel intensity) 437 (95.85, M+H).

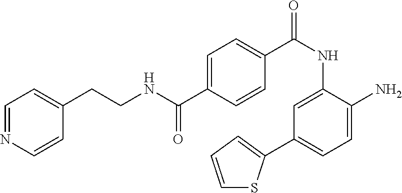

N1-(2-amino-5-(thiophen-2-yl)phenyl)-N4-(2-(pyridin-4-yl)ethyl)terephthalamide (BRD-6597) can be prepared by substituting 2-(4-methylpiperazin-1-yl)ethanamine with 2-(pyridin-4-yl)ethanamine. ESI+ MS: m/z (rel intensity) 443 (97.68, M+H).

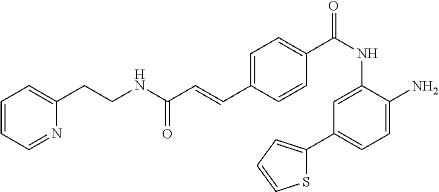

### Synthesis of (E)-N-(2-amino-5-(thiophen-2-yl)phenyl)-4-(3-oxo-3-(2-(pyridin-2-yl)ethylamino)prop-1-enyl)benzamide (BRD-9853)

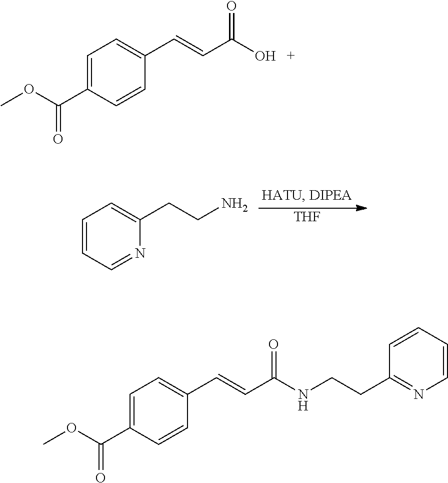

A mixture of (E)-3-(4-(methoxycarbonyl)phenyl)acrylic acid (0.25 g, 1.21 mmol, 1.0 eq), 2-(pyridin-2-yl)ethanamine (0.30 g, 2.42, 2.0 eq), HATU (0.46 g, 1.91, 1.57 eq), DIPEA (0.31 g, 4.36 mmol, 3.6 eq) in THF (10 mL) was stirred at room temperature for 20 h. The reaction was concentrated and ethyl acetate (30 mL) was added. The organic layer was washed with water (20 mL), dried over magnesium sulfate, filtered and concentrated. The product was purified by column chromatography (silica gel, 10% MeOH/CH₂Cl₂) to provide the desired compound (0.20 g, 52% yield). ESI+ MS: m/z (rel intensity) 311 (98.7, M+H).

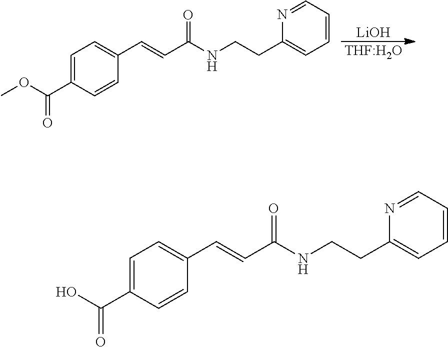

To a solution of (E)-methyl 4-(3-oxo-3-(2-(pyridin-2-yl)ethylamino)prop-1-enyl)benzoate (0.20 g, 0.64 mmol, 1.0 eq) in THF (3 mL) was added a solution of LiOH (0.05 g, 1.93 mmol, 3.0 eq) in water (3 mL). The reaction was stirred at room temperature for 20 h. The reaction was then concentrated and diluted with water (5 mL). The solution was acidified with a 1N aqueous solution of HCl to pH˜2. The precipitate formed was filtered and rinsed with water (3 mL) to afford a white solid (0.15 g, 79% crude yield). ESI+ MS: m/z (rel intensity) 297 (63.8, M+H).

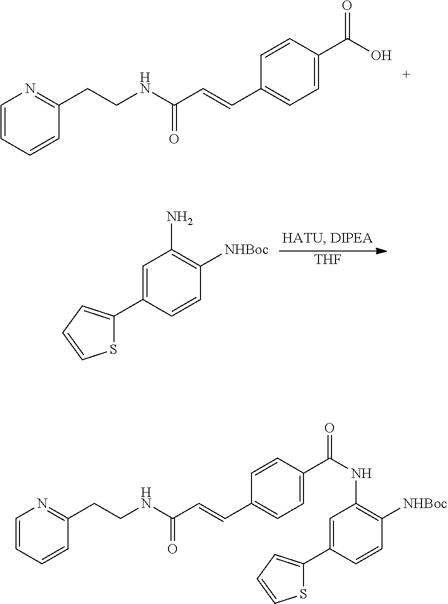

A mixture of (E)-4-(3-oxo-3-(2-(pyridin-2-yl)ethylamino)prop-1-enyl)benzoic acid (0.10 g, 0.34 mmol, 1.0 eq), tert-butyl 2-amino-4-(thiophen-2-yl)phenylcarbamate (0.19 g, 0.68 mmol, 2.0 eq), HATU (0.46 g, 1.91 mmol, 5.6 eq) and DIPEA (0.31 g, 4.36 mmol, 12.9 eq) in THF (10 mL) was stirred at room temperature for 20 h. The reaction was concentrated and ethyl acetate (30 mL) was added. The solution was washed with water (20 mL). The combined organic layers were dried over sodium sulfate, filtered and concentrated. The product was purified by column chromatography (silica gel, 6% MeOH/CH₂Cl₂) to provide the target compound (0.11 g, 56% yield). ESI+ MS: m/z (rel intensity) 569 (98.5, M+H).

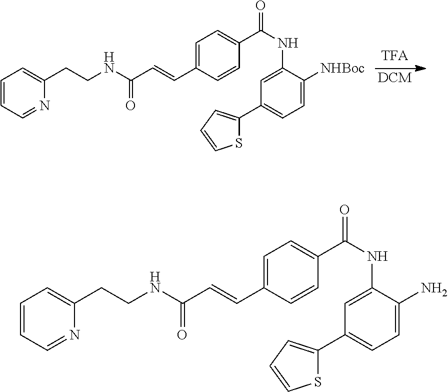

To a solution of (E)-tert-butyl 2-(4-(3-oxo-3-(2-(pyridin-2-yl)ethylamino)prop-1-enyl)benzamido)-4-(thiophen-2-yl)phenylcarbamate (0.11 g, 0.19 mmol, 1.0 eq) in dichloromethane (4 mL) was added trifluoroacetic acid (1.5 mL). The reaction was stirred at room temperature for 1 h and concentrated. The residue was dissolved in ethyl acetate (20 mL), washed with a saturated aqueous solution of sodium bicarbonate (10 mL), water (10 mL). The organic layer was dried over sodium sulfate, filtered and concentrated. The residue was washed with ether (2 mL) to give a yellow solid (0.07 g, 75% yield). ESI+ MS: m/z (rel intensity) 469 (97.9, M+H), ESI+ MS: m/z (rel intensity) 469 (97.9, M+H), ¹H NMR (500 MHz, d⁶-DMSO): δ 9.79 (s, 1H), 8.52 (d, J=4 Hz, 1H), 8.30-8.23 (m, 1H), 8.034 (d, J=7.5 Hz, 2H), 7.75-7.65 (m, 3H), 7.48 (d, J=15 Hz, 2H), 7.36 (d, J=5 Hz, 1H), 7.30 (t, J=8.5 Hz, 2H), 7.26-7.20 (m, 2H), 7.05 (t, J=4.5 Hz, 2H), 6.81 (d, J=8.5 Hz, 1H), 6.74 (d, J=15.5 Hz, 1H), 5.19 (s, 2H), 3.60-3.52 (m, 2H), 2.95 (t, J=7 Hz, 2H).

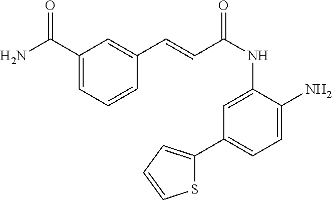

### Synthesis of (E)-3-(3-(2-amino-5-(thiophen-2-yl)phenylamino)-3-oxoprop-1-enyl)benzamide (BRD-3636)

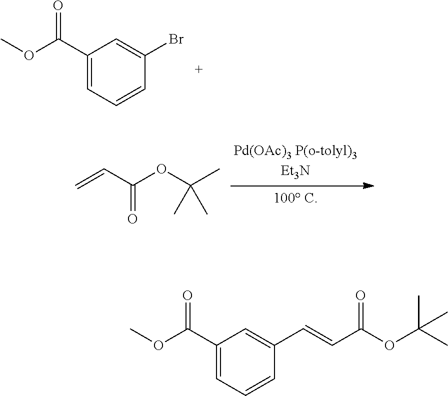

A mixture of methyl 3-bromobenzoate (10.8 g, 50.2 mmol, 1.0 eq), t-butyl acrylate (8.05 g, 62.8 mmol, 1.25 eq), triethylamine (10.16 g, 100 mmol, 2.0 eq), triacetoxylpalladium (0.14 g, 0.50 mmol, 0.01 eq) and tri-o-tolylphosphine (0.61 g, 2.0 mmol, 0.04 eq) was heated at 100° C. for 2 h under nitrogen atmosphere. The reaction mixture was diluted with water. The product was extracted with ethyl acetate. The organic phase was adjusted to pH˜3 with a 1M aqueous solution of HCl. The organic layer was separated, dried over sodium sulfate, filtered and concentrated in vacuo to give a yellow solid (11 g, 84% yield).

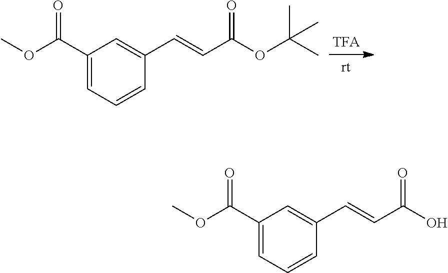

A mixture (E)-methyl 3-(3-tert-butoxy-3-oxoprop-1-enyl)benzoate (12.0 g, 45.7 mmol) in TFA (100 mL) was stirred at room temperature for 20 h. The solvent was removed under reduced pressure. The residue obtained was washed with ethyl acetate to give a white solid (8.5 g, 90% yield).

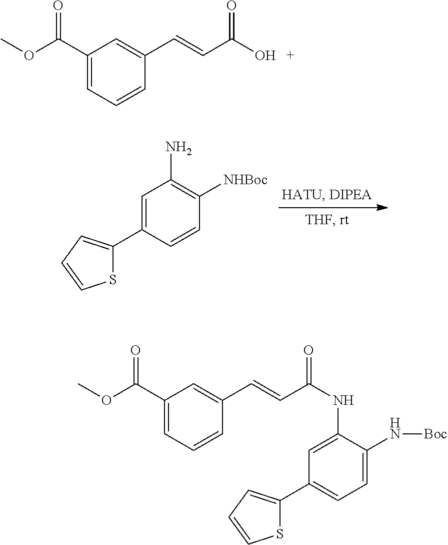

A mixture of (E)-3-(3-(methoxycarbonyl)phenyl)acrylic acid (5.56 g, 27 mmol, 1.5 eq), tert-butyl 2-amino-4-(thiophen-2-yl)phenylcarbamate (5.22 g, 17.98 mmol, 1.0 eq), HATU (10.30, 42.7 mmol, 2.37 eq) and DIPEA (6.96 g, 98 mmol, 5.45 eq) in THF (80 ml) was stirred at room temperature for 20 h. The reaction was then concentrated. The residue was diluted with ethyl acetate and washed with water, then brine. The organic layer was separated, dried over sodium sulfate, filtered and concentrated under reduced pressure. The product was purified by column chromatography (silica gel, 50% PE/CH₂Cl₂) to afford a yellow solid (6.0 g, 64.9% yield).

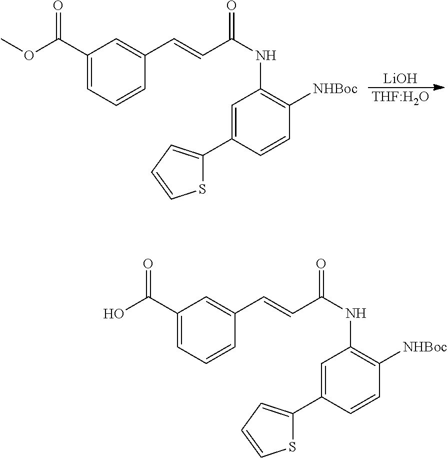

To a solution of (E)-methyl 3-(3-(2-(tert-butoxycarbonylamino)-5-(thiophen-2-yl)phenylamino)-3-oxoprop-1-enyl)benzoate (5.5 g, 11.49 mmol, 1.0 eq) in THF (60 mL) was added a solution of LiOH (0.69 g, 28.7 mmol, 2.5 eq) in water (60 mL). The reaction was stirred at room temperature for 20 h. The reaction was extracted with ethyl acetate. The aqueous layer was separated and acidified with a 1N aqueous solution of HCl to pH˜2. The precipitate formed was filtered and rinsed subsequently with water (200 mL), then methanol (100 mL) to afford a white solid (4.2 g, 79% yield).

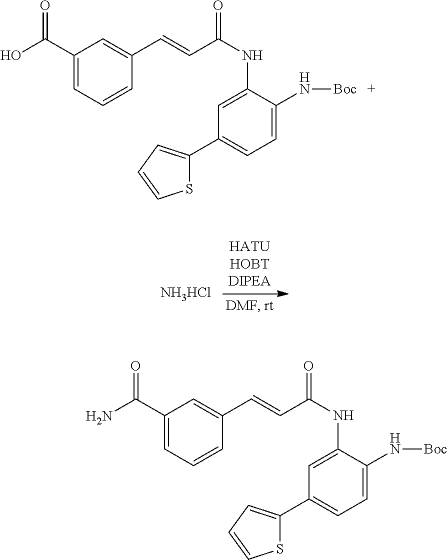

A mixture of ammonia hydrochloride (0.02 g, 0.43 mmol, 2.0 eq), (E)-3-(3-(2-(tert-butoxycarbonylamino)-5-(thiophen-2-yl)phenylamino)-3-oxoprop-1-enyl)benzoic acid (0.10 g, 0.21 mmol, 1.0 eq), HATU (0.08 g, 0.32 mmol, 1.5 eq), HOBt (0.043 g, 0.32 mmol, 1.5 eq) and DIPEA (0.11 g, 0.86 mmol, 4.0 eq) in THF (10 mL) was stirred at room temperature for 20 h. The reaction was then concentrated. The residue was diluted with ethyl acetate and washed with water, then brine. The organic layer was separated, dried over sodium sulfate, filtered and concentrated under reduced pressure. The product was purified by column chromatography (silica gel, 10% MeOH/CH₂Cl₂) to afford the desired product (0.08 g, 80% yield).

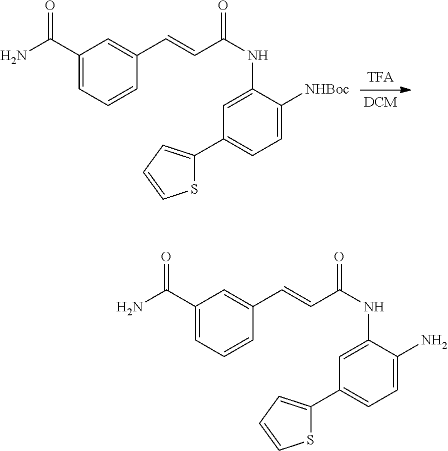

A solution of (E)-tert-butyl 2-(3-(3-carbamoylphenyl)acrylamido)-4-(thiophen-2-yl)phenylcarbamate (0.08 g, 0.17 mmol, 1.0 eq) in CH₂Cl₂ was treated with trifluoroacetic acid (1 mL). The solution was stirred at room temperature for 1 h. The reaction was then concentrated. The residue was dissolved in ethyl acetate (20 mL). The solution was washed with a saturated aqueous solution of sodium bicarbonate (10 mL), then water (10 mL). The combined organic layers were dried over sodium sulfate, filtered and concentrated. The product was washed with ether (2 mL) to give the target compound (0.05 g, 73% yield). ESI+ MS: m/z (rel intensity) 364 (92.63, M+H), ¹H NMR (500 MHz, d⁶-DMSO): δ 9.48 (s, 1H), 8.18 (s, 1H), 8.09 (s, 1H), 7.90 (d, J=8 Hz, 1H), 7.80-7.70 (m, 2H), 7.62 (d, J=16 Hz, 1H), 7.54 (t, J=8 Hz, 1H), 7.49 (s, 1H), 7.36 (d, J=5 Hz, 1H), 7.29-7.19 (m, 2H), 7.10-6.59 (m, 2H), 6.79 (d, J=8 Hz, 1H), 5.24 (s, 2H).

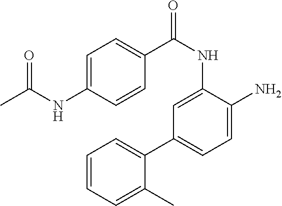

### Synthesis of 4-acetamido-N-(4-amino-2′-methylbiphenyl-3-yl)benzamide (BRD-4029)

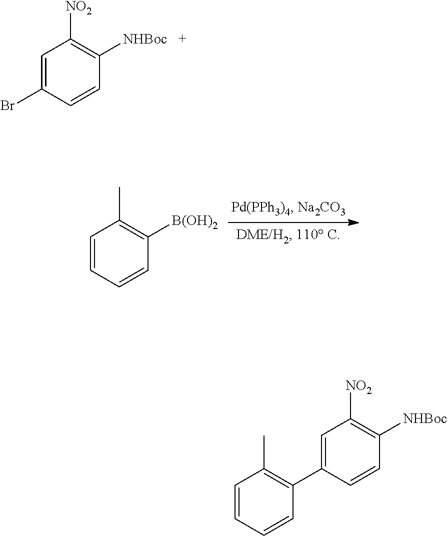

A mixture of tert-butyl 4-bromo-2-nitrophenylcarbamate (0.20 g, 0.62 mmol), o-tolylboronic acid (0.10 g, 0.74 mmol), sodium carbonate (0.20 g, 0.93 mmol) and Pd(PPh₃)₄ (50 mg, 0.04 mmol) in DME/H₂O (2:1, 5 mL) was heated to 110° C. under argon atmosphere. After vigorously stirring for 20 h, water was added. The product was extracted with ethyl acetate. The combined organic layers were washed with water, dried over sodium sulfate, filtered and concentrated. The residue was purified by chromatography (silica gel, 10% EtOAc/PE) to give the desired product as yellow solid (0.13 mg, 95% yield). ¹HNMR (400 MHz, DMSO-d6): 1.46 (s, 9H), 2.25 (s, 3H), 7.26-7.32 (m, 4H), 7.66-7.70 (m, 2H), 7.85 (d, J=1.6 Hz, 1H), 9.67 (s, 1H).

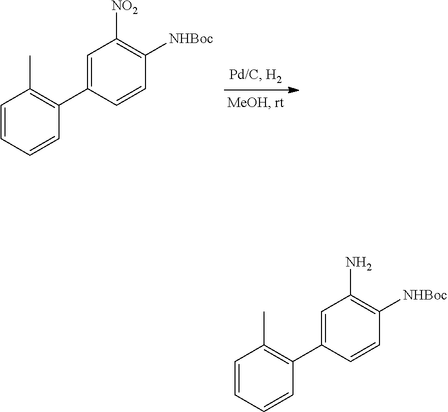

A solution of tert-butyl 2′-methyl-3-nitrobiphenyl-4-ylcarbamate (0.13 g, 0.58 mmol), Pd/C (10%, 0.06 g) in MeOH (5 mL) was vigorously stirred for 16 h under hydrogen atmosphere. The reaction was filtered through Celite. The filtrate was concentrated. The product was purified by column chromatography (silica gel, 2.5% EtOAc/PE) to give the desired product as yellow solid (0.161 g, 92% yield). ¹HNMR (400 MHz, d⁶-DMSO): 1.47 (s, 9H), 2.22 (s, 3H), 4.90 (s, 2H), 6.48 (dd, J=8.0, 1.6 Hz, 1H), 6.64 (d, J=1.6 Hz, 1H), 7.11-7.13 (m, 1H), 7.19-7.25 (m, 4H), 8.34 (s, 1H).

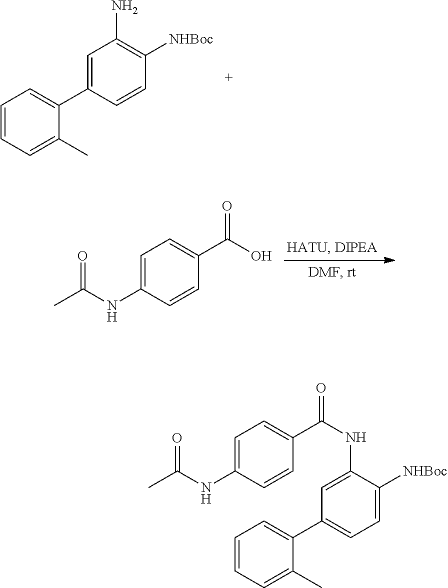

A solution of tert-butyl 3-amino-2′-methylbiphenyl-4-ylcarbamate (0.15 g, 0.508 mmol, 1.0 eq), 4-acetamidobenzoic acid (0.56 g, 3.06 mmol, 6 eq), DIPEA (0.7 mL, 4.08 mmol, 8 eq) and HATU (0.39 g, 1.14 mmol, 2 eq) in DMF (5 mL) was stirred for 16 h under argon atmosphere. Water was added and the product was extracted with EtOAc. The combined organic layers were dried over sodium sulfate, filtered and concentrated. The product was purified by column chromatography (silica gel, 10% EtOAc/PE) to give the desired product as yellow solid (0.10 g, 42% yield). ¹HNMR (400 MHz, d⁶-DMSO): δ 1.47 (s, 9H), 2.08 (s, 3H), 2.28 (s, 3H), 7.16-7.3 (m, 5H), 7.53 (d, J=1.2 Hz, 1H), 7.57 (d, J=8.4 Hz, 1H), 7.72 (d, J=8.8 Hz, 2H), 7.90-7.92 (d, J=8.8 Hz, 1H), 8.80 (s, 1H), 9.80 (s, 1H), 10.24 (s, 1H).

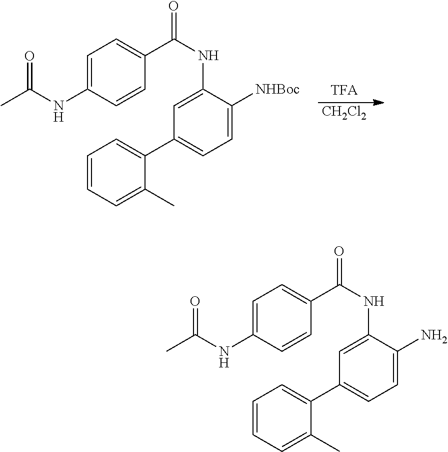

A solution of tert-butyl 3-(4-acetamidobenzamido)-2′-methylbiphenyl-4-ylcarbamate (0.06 g, 0.124 mmol) in CH₂Cl₂ (1.5 mL) was treated with TFA (0.7 mL) at 0° C. After the reaction mixture was stirred at 0° C. for 2 h, the reaction was diluted with ethyl acetate and washed with a saturated solution of sodium bicarbonate. The combined organic layers were dried over sodium sulfate, filtered and concentrated to give the desired product (0.03 g, 47% yield). ¹HNMR (400 MHz, d⁶-DMSO): δ 2.08 (s, 3H), 2.27 (s, 3H), 5.00 (s, 2H), 6.84 (d, J=8.4 Hz, 1H), 6.96 (d, J=8.0, 2.0 Hz, 1H), 7.16-7.25 (m, 5H), 7.69 (d, J=8.8 Hz, 2H), 7.94 (d, J=8.8 Hz, 2H), 9.60 (s, 1H), 10.20 (s, 1H). MS: m/z (360, [M+H]⁺; 382, [M+Na]⁺).

One skilled in the art will recognize that other compounds described below can be prepared in a similar manner to the procedures described above.

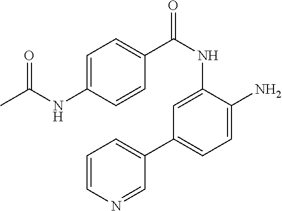

4-acetamido-N-(2-amino-5-(pyridin-3-yl)phenyl)benzamide (ORD-9773) can be prepared by substituting o-tolylboronic acid with pyridin-3-ylboronic acid. ESI+ MS: m/z (rel intensity) 369 (96.2, M+Na).

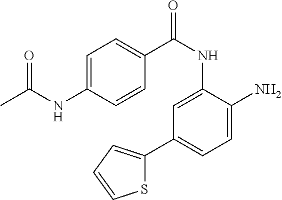

4-acetamido-N-(2-amino-5-(thiophen-2-yl)phenyl)benzamide (BRD-6929) can be prepared by substituting o-tolylboronic acid with thiophen-2-ylboronic acid. ESI+ MS: m/z (rel intensity) 382 (96.2, M+Na).

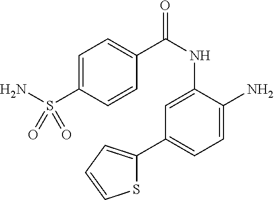

### Synthesis of N-(2-amino-5-(thiophen-2-yl)phenyl)-4-sulfamoylbenzamide (BRD-7726)

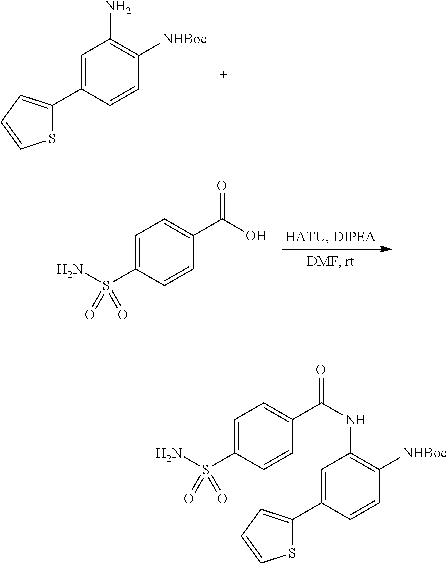

A solution of tert-butyl 2-amino-4-(thiophen-2-yl)phenylcarbamate (0.30 g, 1.03 mmol, 1 eq), 4-sulfamoylbenzoic acid (0.42 g, 2.06 mmol, 2 eq), HATU (780 mg, 2.06 mmol, 2.0 eq.), and DIPEA (0.45 mL, 2.58 mmol, 2.5 eq) in DMF (5 mL) was stirred for 15 h at room temperature. The reaction was quenched with a saturated solution of sodium bicarbonate. The solid obtained was filtered and dried under reduced pressure. The product was purified by column chromatography (silica gel, 3% MeOH/CH₂Cl₂) to afford the desired product (0.25 g, 51% yield).

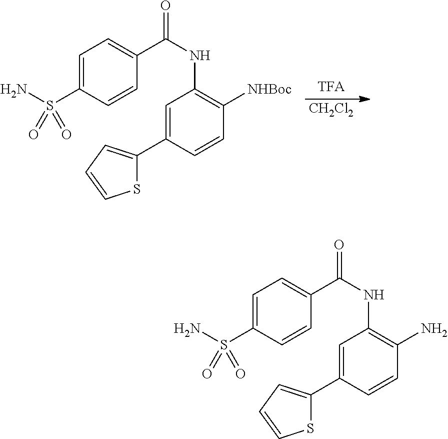

To a stirred solution of tert-butyl 2-(4-sulfamoylbenzamido)-4-(thiophen-2-yl)phenylcarbamate (0.15 g, 0.32 mmol, 1 eq) in CH₂Cl₂ (2 mL) was added TFA (1 mL) at 0° C. The reaction was stirred at room temperature for 2 h. The reaction was then concentrated. The residue was dissolved in EtOAc. The solution was washed with a saturated solution of sodium bicarbonate. The combined organic layers were dried over sodium sulfate, filtered and concentrated. The product was purified by column chromatography (silica gel, 3% MeOH/CH₂Cl₂) to afford the desired product (0.03 g, 24.9% yield). ESI+ MS: m/z (rel intensity) 374 (98.32, M+H), ¹H NMR (500 MHz, d⁶-DMSO): δ 8.15 (d, J=8.5 Hz, 2H), 7.94 (d, J=8 Hz, 2H), 7.49 (s, 1H), 7.36 (d, J=4.5 Hz, 1H), 7.31 (dd, J=8.5, 2 Hz, 1H), 7.44 (d, J=2.5 Hz, 1H), 7.05 (t, J=3.5 Hz, 1H), 6.81 (d, J=8.5 Hz, 1H), 5.21 (s, 2H).

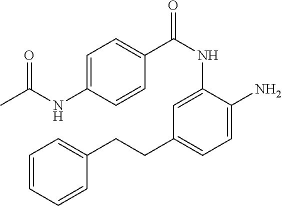

### Synthesis of 4-acetamido-N-(2-amino-5-phenethylphenyl)benzamide (BRD-7050)

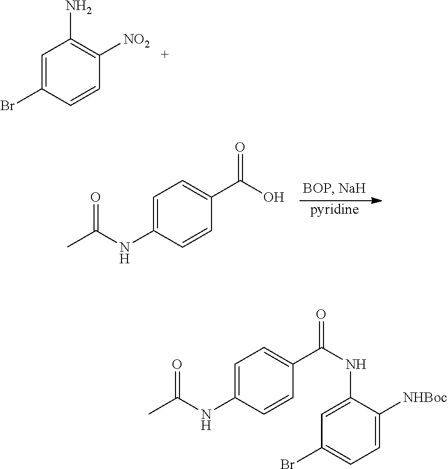

To a stirred solution of 5-bromo-2-nitroaniline (4.0 g, 18.43 mmol, 1.0 eq), 4-acetamidobenzoic acid (4.95 g, 27.6 mmol, 1.5 eq) and BOP (10.60 g, 23.96 mmol, 1.3 eq) was added sodium hydride (2.96 g, 123.0 mmol, 6.7 eq) portion-wise at 0° C. The reaction mixture was allowed to warm to room temperature and stir for 60 h. The solvents were evaporated under reduced pressure. The residue was diluted with a saturated solution of sodium bicarbonate. The obtained precipitate was filtered. The product was purified by column chromatography (silica gel, 25% EtOAc/CH₂Cl₂) to afford the desired product (3.31 g, 40% yield).

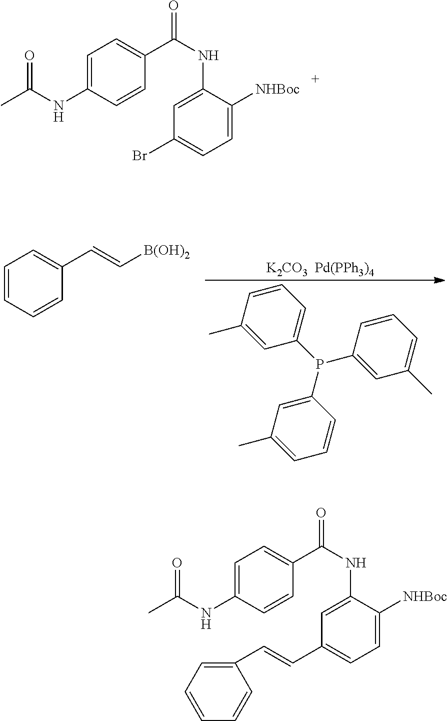

A mixture of tert-butyl 2-(4-acetamidobenzamido)-4-bromophenylcarbamate (0.50 g, 1.11 mmol, 1.0 eq), (E)-styrylboronic acid (0.33 g, 2.23 mmol, 2.0 eq), potassium carbonate (0.46 g, 3.335 mmol, 3.0 eq), Pd(PPh₃)₄ (0.09 g, 0.08 mmol, 0.07 eq) and tritolyl phosphine (0.10 g, 0.33 mmol, 0.3 eq) in DME/H₂O (30 mL) was heated to reflux for 20 h. The reaction mixture was diluted with water. The obtained solid was filtered. The crude product was purified by column chromatography (silica gel, 2% MeOH/CH₂Cl₂) to obtain pure product (0.30 g, 57% yield).

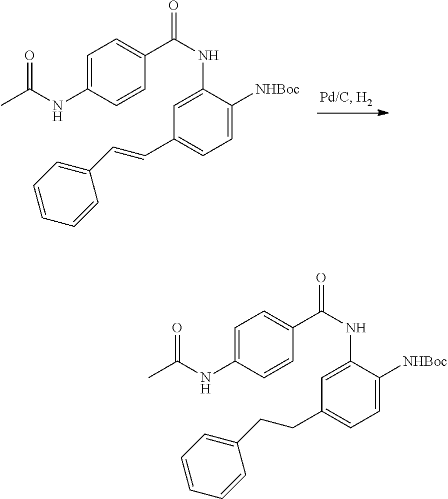

To a solution of (E)-tert-butyl 2-(4-acetamidobenzamido)-4-styrylphenylcarbamate (0.15 g, 0.32 mmol, 1.0 eq) in ethanol (10 mL) was added palladium on carbon (0.02 g, 0.23 mmol, 0.7 eq). The reaction mixture was stirred under H₂ atmosphere for 20 h. The reaction mixture was filtered through celite, the solids were washed with methanol. The reaction was then concentrated under reduced pressure to afford an off white solid (0.14 g, 93% crude yield) which was used in the next step without further purification.

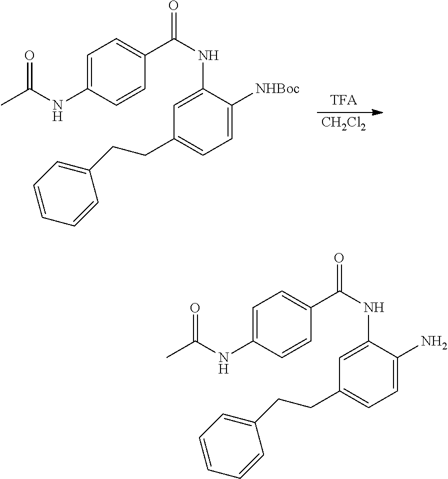

To a solution of tert-butyl 2-(4-acetamidobenzamido)-4-phenethylphenylcarbamate (0.14 g, 0.29 mmol) in CH₂Cl₂ (3 mL) at 0° C. was added TFA (2 mL) dropwise. The reaction mixture was slowly warmed to room temperature and stirred for 2 h. The solvent was removed by evaporation under reduced pressure. The crude residue was diluted with water and quenched with a saturated aqueous solution of sodium bicarbonate. The obtained solid was filtered, washed with water and dried under vacuum to afford the desired product (0.08 h, 68% yield). ESI+ MS: m/z (rel intensity) 374 (95.0, M+H).

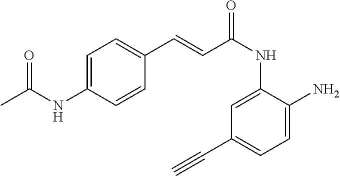

### Synthesis of (E)-3-(4-acetamidophenyl)-N-(2-amino-5-ethynylphenyl)acrylamide (BRD-0063)

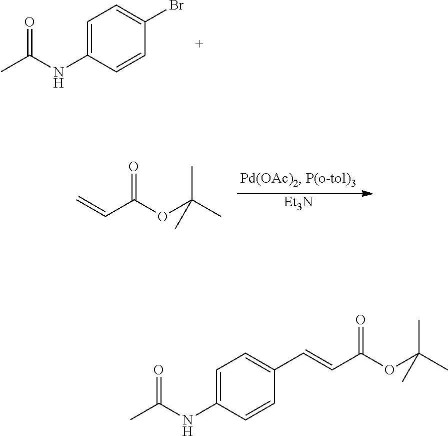

A mixture of N-(4-bromophenyl)acetamide (2.14 g, 10 mmol, 1.0 eq), tert-butyl acrylate (1.6 g, 13 mmol, 1.3 eq), diacetoxypalladium (0.05 g, 0.2 mmol, 0.02 eq), P(o-tol)₃ (0.12 g, 0.4 mmol, 0.04 eq) in triethylamine (3 mL) was heated to 100° C. for 2 h under nitrogen. The reaction was cooled to room temperature. Ethyl acetate (50 mL) was added. The organic layer was washed with water (2×20 mL). The combined organic layers were dried over sodium sulfate, filtered and concentrated to give the desired product (2.2 g, 76% crude yield) as a yellow solid.

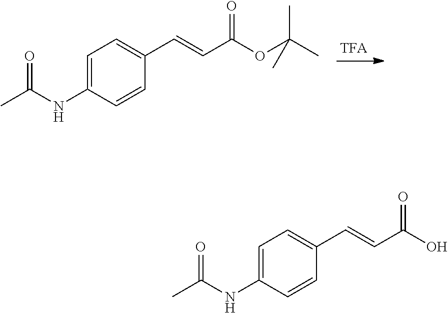

(E)-tert-butyl 3-(4-acetamidophenyl)acrylate (2.2 g, 8.42 mmol) in trifluoroacetic acid (10 mL) was stirred at room temperature for 10 min. The solvent was removed by evaporation. The residue was dissolved in aqueous sodium carbonate (0.3N, 30 mL). The aqueous layer was washed with ethyl acetate (2×20 ml), acidified to pH˜3 with a 1N aqueous solution of hydrochloride acid. The product was extracted with ethyl acetate (50 mL). The combined organic layers were dried over sodium sulfate, filtered and concentrated to get the desired product (1.4 g, 77% crude yield).

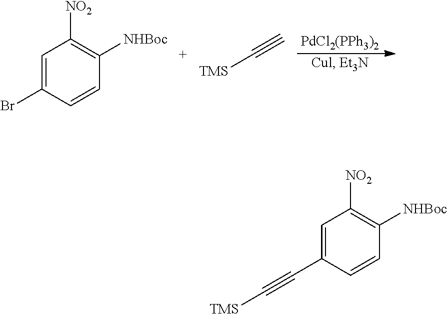

A mixture of tert-butyl 4-bromo-2-nitrophenylcarbamate (0.97 g, 3.08 mmol, 1.0 eq), ethynyltrimethylsilane (0.45 g, 4.62 mmol, 1.5 eq), PdCl₂(PPh₃)₂ (0.11 g, 0.15 mmol, 0.05 eq) and CuI (0.04 g, 0.18 mmol, 0.06 eq) in Et₃N (35 mL) was refluxed at 100° C. for 2 h. The reaction was cooled to room temperature and the solvent was removed by evaporation. The residue was taken up in water (50 mL) and ethyl acetate (50 mL). The organic layer was dried over sodium sulfate, filtered and concentrated to get the crude product (1.2 g, 116% crude yield) as yellow oil which was used without further purification in the next step.

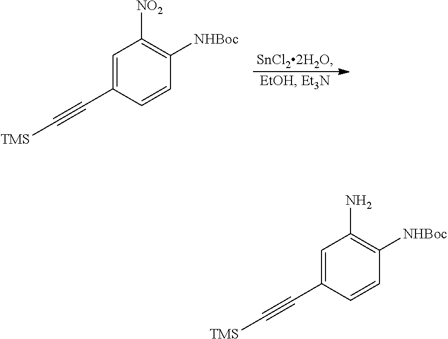

A mixture of tert-butyl 2-nitro-4-((trimethylsilyl)ethynyl)phenylcarbamate (1.2 g, 3.59 mmol, 1.0 eq), SnCl₂.2H₂O (4.05 g, 17.94 mmol, 5.0 eq) and Et₃N (15 mL) in ethanol (30 mL) was heated to 70° C. for 1 h. The reaction was then cooled to room temperature. The solvents were removed by evaporation. The residue was taken up in water (50 mL) and ethyl acetate (50 mL). The organic layer was dried over sodium sulfate, filtered and concentrated. The crude product was purified by column chromatography (silica gel, 25% EtOAc/PE) to afford the desired product (0.58 g, 53% yield) as yellow solid.

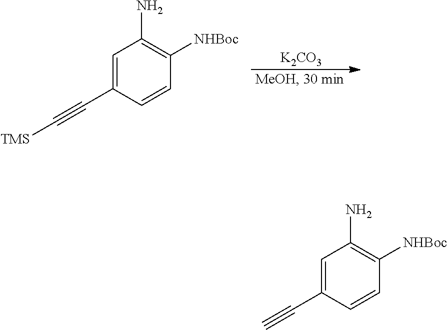

A solution of tert-butyl 2-amino-4-((trimethylsilyl)ethynyl)phenylcarbamate (0.20 g, 0.65 mmol, 1.0 eq) in methanol (10 mL) was treated with K₂CO₃ (0.45 g, 3.28 mmol, 5.0 eq). The reaction was stirred at room temperature for 30 min. The solvent was evaporated and the residue was taken up in water (20 mL) and ethyl acetate (30 mL). The organic layer was dried over sodium sulfate, filtered and concentrated to get the desired product (0.14 g, 89% yield) which was used without further purification in the next step.

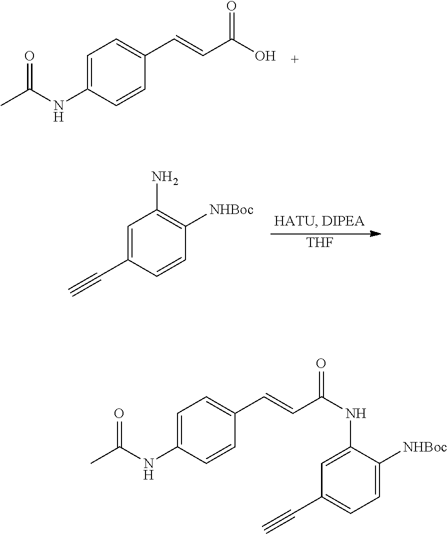

A mixture of (E)-3-(4-acetamidophenyl)acrylic acid (0.14 g, 0.70 mmol, 1.2 eq), tert-butyl 2-amino-4-ethynylphenylcarbamate (0.13 g, 0.58 mmol, 1.0 eq), HATU (0.26 g, 0.70 mmol, 1.2 eq) and DIPEA (0.23 g, 1.75 mmol, 3.0 eq) in THF (10 mL) was stirred at room temperature for 20 h. The solvents were evaporated under reduced pressure. The residue was diluted with water (20 mL). The product was extracted with ethyl acetate (20 mL) twice. The combined organic layers were washed with brine, dried over sodium sulfate, filtered and concentrated. The product was purified by column chromatography (silica gel, 6% MeOH/CH₂Cl₂) to afford the desired product (0.12 g, 49% yield).

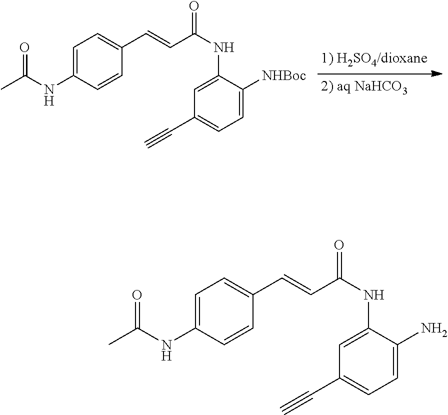

A solution of (E)-tert-butyl 2-(3-(4-acetamidophenyl)acrylamido)-4-ethynylphenylcarbamate (0.06 g, 0.14 mmol) in 1,4 dioxane (1 mL) at room temperature was treated with a solution of H₂SO₄ (0.10 g, 7.15 mmol, 50 eq) in 1,4 dioxane (1 mL). The resulting mixture was stirred at room temperature for 2 h. The reaction mixture was quenched with a saturated aqueous solution of sodium bicarbonate. The product was extracted with ethyl acetate. The combined organic layers was dried over sodium sulfate, filtered and concentrated. The product was purified by column chromatography (silica gel, 6% MeOH/CH₂Cl₂) to afford the desired product (0.01 g, 21% yield). ESI+ MS: m/z (rel intensity) 319 (94.13, M+H).

All references, patents and patent publications that are recited in this application are incorporated in their entirety herein by reference.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

What is claimed is:

1. A method for enhancing a memory in a subject comprising administering to the subject an HDAC2 inhibitor in an amount effective to enhance the memory in the subject, wherein the HDAC2 inhibitor is a selective HDAC2 inhibitor.

2-7. (canceled)

8. The method of claim 1, wherein a synaptic network in the subject is re-established.

9. The method of claim 1, wherein the HDAC2 inhibitor is not an HDAC1 inhibitor.

10. The method of claim 1, wherein the HDAC2 inhibitor is not an HDAC5, HDAC6, HDAC7 or HDAC10 inhibitor.

11-12. (canceled)

13. The method of claim 1, wherein the selective HDAC2 inhibitor is an HDAC2 RNAi such as a siRNA, shRNA, miRNA, dsRNA or ribozyme or variants thereof.

14-22. (canceled)

23. A method for treating Alzheimer's disease comprising, administering to a subject having Alzheimer's disease an HDAC2 inhibitor in an amount effective to treat Alzheimer's disease, wherein the HDAC2 inhibitor is a selective HDAC2 inhibitor.

24. (canceled)

25. The method claim 23 wherein the HDAC2 inhibitor is a selective HDAC1/HDAC2 inhibitor.

26. The method of claim 25, wherein the HDAC2 inhibitor is a compound of formula (IV) 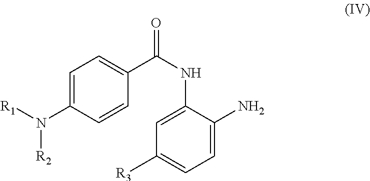
wherein
R₁ and R₂ are independently selected from H, and —C(O)—C₁₋₆alkyl;
R₃ is optionally substituted aryl, optionally substituted heteroaryl, or aryl-C₁₋₆alkylene.

27. The method of claim 26, wherein formula IV is

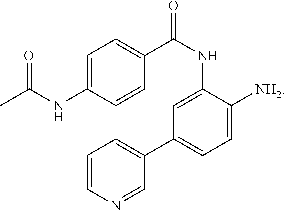

28. The method of claim 26, wherein formula IV is

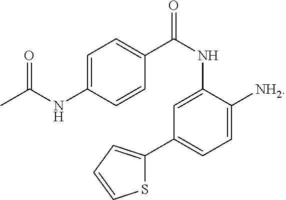

29. The method of claim 26, wherein formula IV is

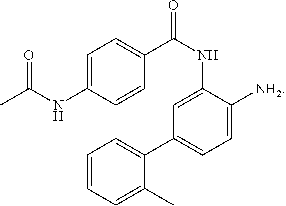

30. The method of claim 26, wherein formula IV is

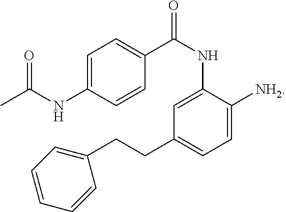

31. The method of claim 23 wherein the HDAC2 inhibitor is a selective HDAC1/HDAC2/HDAC3 inhibitor.

32. The method of claim 31, wherein the HDAC2 inhibitor is a compound of formula (VI) 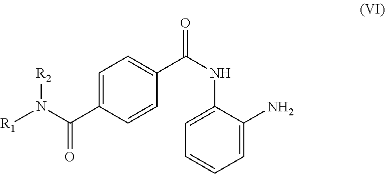
wherein
R₁ and R₂ are independently selected from H, substituted or unsubstituted, branched or unbranched, cyclic or acyclic C₁₋₆alkyl, heterocyclyl, heteroaryl, aryl, and aryl-C₁₋₆alkylene.

33. The method of claim 32, wherein formula VI is

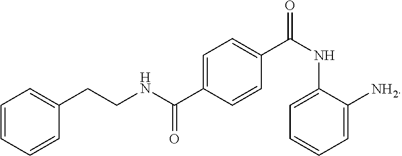

34. The method of claim 23, wherein the HDAC2 inhibitor is a compound of formula (I) 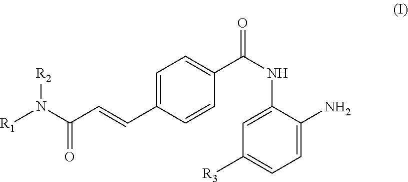
wherein
R₁ and R₂ are independently selected from H, substituted or unsubstituted, branched or unbranched, cyclic or acyclic C₁₋₆alkyl, heterocyclyl, C₁₋₆alkylene, heteroaryl, heteroarylene, and heteroarylene-alkylene; and
R₃ is aryl or heteroaryl.

35. The method of claim 34, wherein formula I is

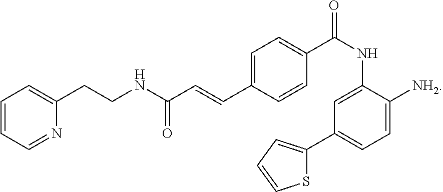

36. The method of claim 23, wherein the HDAC2 inhibitor is a compound of formula (II) 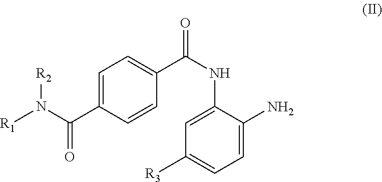
wherein
R₁ and R₂ are independently selected from H, substituted or unsubstituted, branched or unbranched, cyclic or acyclic C₁₋₆alkyl, heterocyclyl, C₁₋₆alkylene, heteroaryl, heteroarylene, heteroarylene-alkylene, arylene-alkylene; and heterocyclyl-alkylene optionally substituted; and
R₃ is aryl or heteroaryl.

37. The method of claim 36, wherein formula II is

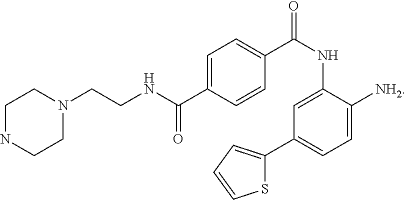

38. The method of claim 36, wherein formula II is

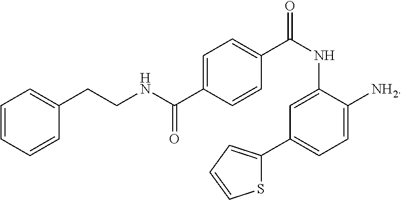

39. The method of claim 36, wherein formula II is

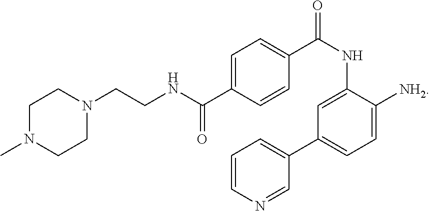

40. The method of claim 36, wherein formula II is

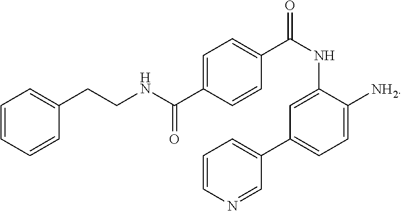

41. The method of claim 36, wherein formula II is

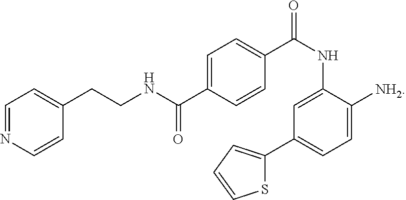

42. The method of claim 23, wherein the HDAC2 inhibitor is a compound of formula (III) 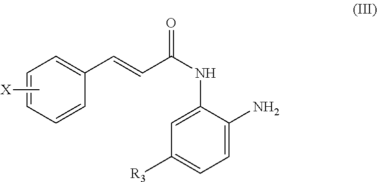
wherein
X is —C(O)—N(R₁)(R₂), C₁₋₆alkylene-N(H)—C₁₋₆alkylene-N(R₁)C(O)(R₂); or —N(R₁)C(O)R₂; R₁ and R₂ are independently selected from H, and substituted or unsubstituted, branched or unbranched, cyclic or acyclic C₁₋₆alkyl; and
R₃ is alkynyl, aryl, or heteroaryl.

43. The method of claim 42, wherein formula III is

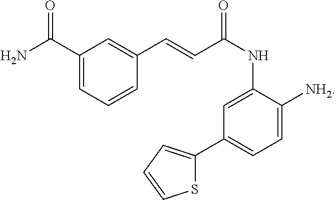

44. The method of claim 42, wherein formula III is

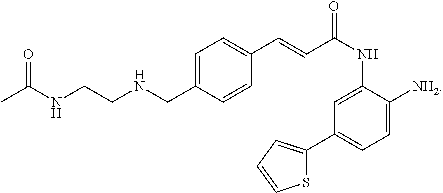

45. The method of claim 42, wherein formula III is

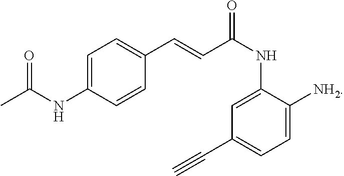

46. The method of claim 23, wherein the HDAC2 inhibitor is a compound of formula (V) 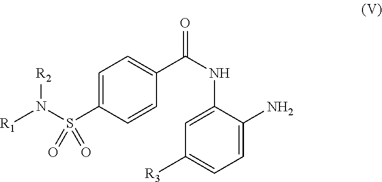
wherein
R₁ and R₂ are independently selected from H, and substituted or unsubstituted, branched or unbranched, cyclic or acyclic C₁₋₆alkyl; and
R₃ is aryl or heteroaryl.

47. The method of claim 46, wherein formula V is

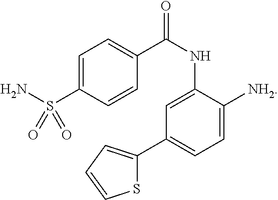

48-51. (canceled)

52. The method of claim 23 wherein the HDAC2 inhibitor is a selective HDAC1/HDAC2/HDAC10 inhibitor.

53. The method of claim 23 wherein the HDAC2 inhibitor is a selective HDAC1/HDAC2/HDAC3/HDAC10 inhibitor.
